(19) United States
(12) Patent Application Publication
(10) Pub. No.: US 2021/0069295 A1
(43) Pub. Date: Mar. 11, 2021

(54) GALECTINS CONTROL MTOR IN RESPONSE TO ENDOMEMBRANE DAMAGE AND PROVIDE A MECHANISM AND TARGET FOR THE TREATMENT OF AUTOPHAGY-RELATED DISEASES

(71) Applicant: STC. UNM, Albuquerque, NM (US)

(72) Inventors: Vojo P. Deretic, Placitas, NM (US); Jingyue Jia, Albuquerque, NM (US)

(21) Appl. No.: 16/977,318

(22) PCT Filed: Nov. 9, 2018
(86) PCT No.: PCT/US2018/060020

§ 371 Date: Sep. 1, 2020

Related U.S. Application Data
(60) Provisional application No. 62/584,486, filed on Nov. 10, 2017.
(60) Provisional application No. 62/651,388, filed on Apr. 2, 2018.

(51) Int. Cl. A61K 38/17; A61K 31/4706; A61K 38/05; A61K 31/133; A61K 31/16; A61K 31/4164; A61K 31/7016; C07K 16/28; A61K 31/732; A61K 31/4745; A61K 31/52; A61K 31/436; A61K 31/675; A61K 31/437; A61K 31/353; A61K 31/522; A61K 31/12; A61K 31/05

(52) U.S. Cl. CPC A61K 38/1732; C07K 2317/21; A61K 38/05; A61K 31/133; A61K 31/16; A61K 31/4164; A61K 31/7016; C07K 16/2818; A61K 31/732; A61K 31/4745; A61K 31/52; A61K 31/436; A61K 31/675; A61K 31/437; A61K 31/353; A61K 31/522; A61K 31/12; A61K 31/05; A61K 31/4706

(57) ABSTRACT

The present invention is directed to the discovery that Galectins and in particular, Galectin-8 and Galectin-9 control mTor response (Galectin-8 is a mTOR inhibitor and Galectin-9 is modulator/upregulator of AMPKinase) to endomembrane damage and these compositions can be used, either alone or together, optionally in combination with a lysomotropic agent and other bioactive agents as compositions for the treatment of autophagy-elated diseases. The present invention is directed to pharmaceutical compositions and methods for treating autophagy-related diseases as described herein.

### RELATED APPLICATIONS AND GRANT SUPPORT

This application claims the benefit of priority of provisional applications serial nos. U.S. 62/584,486, filed 10 Nov. 2017 and U.S. 62/651,388, filed 2 Apr. 2018, both of identical title to the present application, the entirety of both of which applications is incorporated by reference herein.

This invention was made with government support under Grant Nos. P20 GM121176, R01 AI042999 and R01 AI111935, each awarded by the National Institutes of Health (NIH). The government has certain rights in the invention.

### FIELD OF THE INVENTION

The present invention is directed to the discovery that Galectins and in particular, Galectin-8 and Galectin-9 control mTor response (Galectin-8 is a mTOR inhibitor and Galectin-9 is modulator/upregulator of AMPKinase) to endomembrane damage and these compositions can be used, either alone or together, optionally in combination with a lysomotropic agent and other bioactive agents as compositions for the treatment of autophagy-related diseases. The present invention is directed to pharmaceutical compositions and methods for treating autophagy-related diseases as described herein which are also useful for targeting the newly identified molecular complex referred to as GALTOR.

### BACKGROUND AND OVERVIEW OF THE INVENTION

The Ser/Thr protein kinase mTOR controls metabolic pathways, including the catabolic process of autophagy. Autophagy plays additional, catabolism-independent roles in homeostasis of cytoplasmic endomembranes and whole organelles. How signals from endomembrane damage are transmitted to mTOR to orchestrate autophagic responses is not known. Here we show that mTOR is inhibited by lysosomal damage. Lysosomal damage, recognized by galectins, leads to association of Gal8 with mTOR apparatus on the lysosome. Gal8 inhibits mTOR activity through its Ragulator-Rag signaling machinery. Thus, a novel galectin-based signal-transduction apparatus, termed here GALTOR, controls mTOR in response to lysosomal damage.

Cellular responses to changing metabolic and energy states are under the control by the Ser/Thr protein kinases mTOR (Saxton and Sabatini, 2017) and AMPK (Garcia and Shaw, 2017), which orchestrate anabolic and catabolic pathways including the autophagosomal-lysosomal system (Mizushima, et al., 2011). AMPK and mTOR reciprocally control autophagy when cells starve. mTOR acts as a negative regulator by phosphorylating inhibitory sites on regulators of autophagy including ULK1 (Kim, ET AL., 2011) as well as on MiT/TFE family factors including TFEB, a transcriptional regulator of the lysosomal system (Napolitano and Ballabio, 2016). In contrast, AMPK promotes autophagy by phosphorylation of activating sites on autophagy factors including ULK1 (Kim, et al., 2011). AMPK and mTOR circuitry overlap, as AMPK inhibits mTOR (Gwinn, et al., 2008; Shaw, et al., 2004).

Active mTOR localizes to several intracellular compartments (Betz and Hall, 2013) including lysosomes where sensory systems control activity of mTOR-Raptor containing complexes termed mTORC1 (Saxton and Sabatini, 2017). Lysosomal location allows mTOR to integrate signals coming from nutrients (e.g. amino acids and cholesterol) via Rag GTPases and their guanine nucleotide exchange factor (GEF) Ragulator with signals from growth factors via Rheb GTPase (Castellano, ET AL., 2017; Demetriades, et al., 2014; Sacton and Sabatini, 2017). Rheb is inhibited by GTPase activating protein (GAP) tuberous sclerosis complex (TSC1/TSC2) in the absence of growth factors (Saxton and Sabatini, 2017), with noted overlaps between growth factors and amino acid sensing (Carroll, et al., 2016). Rag heterodimer pairs, comprised of RagA/B/C/D, respond to availability of amino acids (Saxton and Sabatini, 2017), and cholesterol (Castellano, et al., 2017). mTOR is recruited to lysosomes via Rags (Sancak, et al., 2008), when RagA/B are loaded with GTP through the action of the cognate GEF, a pentameric complex of LAMTOR1-5 (e.g. LAMTOR1/p18, LAMTOR2/p14, etc.) collectively termed “Ragulator” (Bar-Peled, et al., 2012). The Ragulator-Rag complex (Sancak, et al., 2010) cooperates with vacuolar H ATPase (Zoncu, et al., 2011) and this mega-complex interacts with the lysosomal amino acid transporter SLC38A9 (Jung, et al., 2015; Rebsamen, et al., 2015; Wang, et al., 2015). SLC38A9 activates Ragulator in response to lysosomal arginine (Saxton and Sabatini, 2017) or lysosomal cholesterol (Castellano, 2017). Affinities between different components change in response to inputs, e.g. nutrients such as amino acids or cholesterol activate Ragulator and Rags, reflected in weakening of the interactions between components of the GEF Ragulator complex (e.g. p14) and RagA/B due to increased GTP loading of RaA/B, which, as expected, diminishes their affinity for the congnate GEF (Castellano, et al., 2017). As a result, mTOR activity increases as evidenced by phosphorylation of targets such as S6K, 4EBP and ULK1 (Saxton and Sabatini, 2017).

The above processes are complemented by the action of AMPK (Garcia and Shaw, 2017). AMPK directs changes in metabolism under conditions of low energy charge (Garcia and Shaw, 2017). AMPK activates TSC2 (Shaw, et al., 2004), a GAP for Rheb, and phosphorylates negative regulatory sites on Raptor (Gwinn, et al., 2008), a key mTOR adaptor for upstream regulators and effectors, and thus acts as a negative regulator of mTOR. These antagonistic intersections between mTOR and AMPK in metabolic regulation are reflected in their effects on autophagy.

Autophagy differs from other nutritional responses in that it also plays a key role in cytoplasmic quality control (Mizushima, et al., 2011). Autophagy removes protein aggregates (Johansen and Lamark, 2011) and dysfunctional or disused organelles, e.g. lysosomes (Chauhan, et al., 2016; Fujita, et al., 2013), mitochondria (Lazarou, et al, 2015), peroxisomes (Deosaran, et al., 2013; Zhang, et al., 2015), ER (Khaminets, et al., 2015), etc. How mTOR and AMPK are integrated with the quality control functions of autophagy is not well understood. Lysosomal and phagosomal damage are used as a model to study quality control functions of autophagy in cytoplasmic endomembrane maintenance. It has been shown that cytosolic lectins, galectins, can recognize membrane damage by binding to β-galactosides on exofacial (lumenal) membrane leaflets following damage. Galectins form intracellular puncta in response to lysosomal damaging agents such as polymers of Leu-Leu-OMe (LLOMe) (Aits, et al., 2015; Thiele and Lipsky, 1990) or glycyl-L-phenylalanine 2-naphththylamide (GPN) (Berg, et al., 1994), poking membrane holes, action of bacterial secretory systems permeabilizing vacuoles (Thurston, et al., 2012), or effects of inanimate objects (Fujita, et al., 2013). In all studies carried out to date the paradigm has been that galectins, e.g. galectin-3 (Gal3) and galectin-8 (Gal8), recognize membrane damage by binding to lumenal R-galactosides once glycoconjugates on exofacial leaflet are exposed to the cytosol, and bind to and recruit autophagic receptors, e.g. NDP52 in the case of Gal8 (Thurston, et al., 2012) or TRIM16 in the case of Gal3 (Chauhan, et al., 2016). The receptors in turn bind to mammalian Atg8 paralogs to deliver cargo to autophagosomes (Chauhan, et al., 2016; Fujita, et al., 2013; Thurston, et al., 2012). Very little is known whether these membrane damage recognition systems cooperate with mTOR and other signaling to activate autophagy. There are indications that lysosomal damage by GPN (Manifava, et al., 2016 or LLOMe (Chauhan, et al., 2016) may decrease mTOR activity. However, the mechanism for how membrane damage is recognized and transduced to mTOR has not been defined. Since it is surprisingly insensitive to proton gradient dissipation (Zoncu, et al., 2011) it is not trivial to predict consequences of physical damage to lysosomal membranes on mTOR activity.

In this application the inventors evidence a direct role of Gal8 in control of mTOR, and show evidence for control of AMPK by galectin-9 (Gal9), beyond the concept of passive contributions of galectins as simple tags marking the damaged lysosomes and phagosomes for selective autophagy (Fujita, et al., 2013; Thurston, et al., 2012). The work described herein uncovers surprising physical and regulatory relationships between Gal8 and mTOR in the context of endomembrane damage. This represents a paradigm shift in term of how the art presently thinks galectins work in autophagy, provides a quality control physiological input for mTOR, i.e. lysosomal damage, and delineates how this signal is transduced to mTOR and to its downstream effector targets and processes.

### SUMMARY OF THE INVENTION

The present invention is directed to the discovery that Galectins and in particular, Galectin-8 and Galectin-9 may be used alone or in combination and optionally in combination with at least one lysosomotropic agent and/or an autophagy modulator agent for treatment of autophagy-related disease states, disorders and/or conditions. It has been discovered that Galectin-9 is a mTOR inhibitor and Galectin-9 upregulates AMPKinase, the result being that either of these agents alone or together are particularly effective in treating autophagy disease states, disorders and/or conditions, especially when these agents are combined with at least one lysosomotropic agent. In certain embodiments, Galectin-8, Galectin-9 or Galectin-8 and Galectin-9 may be combined with galactose or a related agent and/or at least one lysomotropic agent to enhance the therapeutic effect in the treatment of autophagy-related disease states and/or conditions. In alternative embodiments, galactose or a related agent which functions similarly to galectin-8 as an inhibitor of mTOR or an agent which functions similarly to galectin-9 as an agonist/upregulator of AMPKinase may be used in combination with at least one lysosomotropic agent in pharmaceutical compositions for the treatment of an autophagy-related disease state or condition as described herein. In alternative embodiments, an upregulator of galectin-8 or galectin-9 may be used in combination with a lysosomotropic agent for the treatment of a lysosomal related disease state or condition. These agents which upregulate galectin-8 or galectin-9 are sugars which comprises at least one galactose unit, a sugar selected from a monosaccharide, including β-galactoside sugars, such as galactose, including N- or O-linked galactosides and disaccharides, oligosaccharides and polysaccharides which contain at least one galactose unit. In particular aspects the sugar is galactose, a galactoside, lactose, mannobiose, melibiose, melibiulose (which may have the galactose residue optionally N-acetylated), rutinose, rutinulose, xylobiose, and trehalose, all of which optionally comprise N and O-linked acetyl groups, or an oligosaccharide containing at least one galactose unit, such as a galactooligosaccharide ranging from three to about ten-fifteen galactose units in size, or the sugar is a galactoside or is a galactose derivative, or a lipoarabinomaman or its derivatives. In still additional embodiments, compositions according to the present invention may include an optional autophagy modulator as a bioactive agent.

In embodiments, the present invention is directed to a method of treating an autophagy mediated disease in a patient in need comprising administering to said patient an effective amount of Galectin-8 and/or Galectin-9, a modulator/upregulator of Galectin-8 and/or Galectin-9, or an agent which acts similar to Galectin-8 as an inhibitor of mTOR and/or Galectin-9 as a modulator (upregulator) of AMPKinase or a mixture thereof, optionally in combination with a lysosomotropic agent.

In other embodiments, the present invention is directed to a pharmaceutical composition comprising an effective amount of Galectin-8 and/or Galectin-9, a modulator/upregulator of Galectin-8 and/or Galectin-9, or an agent which acts similar to Galectin-8 as an inhibitor of mTOR and/or Galectin-9 as a modulator (upregulator) of AMPKinase or a mixture thereof, optionally in combination with a lysosomotropic agent.

### BRIEF DESCRIPTION OF THE FIGURES

FIG. 1 shows lysosomal damage inhibits mTOR signaling. (A) Dose-response analysis of mTOR activity in HEK293T cells treated with glycyl-L-phenylalanine 2-naphththylamide (GPN) in full medium for 1 h. mTOR activity was monitored by inununoblotting analysis of S6K1 (T389) and ULK1 (S757) phosphorylation (phosphorylated S6K (T389) and ULK1 (S757) relative to total S6K and ULK1, respectively). (B) Analysis of mTOR activity (as in A) after GPN washout. HEK293T cells were treated with 100 μM GPN for 1 h followed by 1 h washout in full medium. Data, means±SEM (n=3), **p<0.01, ANOVA. (C) Analysis of mTOR activity (as in A) in HEK293T cells treated with increasing doses of silica in full medium for 1 h. (D) HEK293T cells were treated with lysosomal damaging agents (LLOMe, Leu-Leu-OMe) for 1 h in full medium and status of acidified organelles assessed by quantifying LysoTracker Red DND-99 puncta using automated high-content imaging and analysis (HC). None-treated cells were as control (Ctrl). White masks, algorithm defined cell boundaries (primary objects); yellow masks, computer-identified LysoTracker Red puncta (target objects). Data, means±SEM, n≥3 independent experiments (500 primary objects counted per well; ≥5 wells/sample per each experiment), **p<0.01, ANOVA. (E) Analysis of mTOR activity in primary human macrophages treated with GPN. Human peripheral blood monocyte derived macrophages were treated with 100 μM GPN in full medium or starved in EBSS for 1 h, and mTOR activity measured as in A. None-treated cells were as control (Ctrl). (F) Quantification by HC of overlaps between mTOR and LAMP2 (representative images shown in FIG. S1D) in HeLa cells treated with lysosomal damaging agents for 1 h in full medium. Data, means±SEM, n≥3 independent experiments (500 primary objects counted per well; ≥5 wells/sample per each experiment) **p<0.01, ANOVA. (G) Immunofluorescence confocal microscopy visualization of mTOR localization relative to LAMP2-positive lysosomes. HeLa cells were treated with 100 μM GPN in full medium for 1 h, followed by immunostaining of endogenous LAMP2 (green florescence, Alexa-488) and mTOR (red florescence, Alexa-568). Scale bar, 5 sm. (H) Analysis of TFEB nuclear translocation in HeLa cells treated with 100 μM GPN in full medium for 1 h, and the nuclear translocation of TFEB was measured by HC (Nuclei, Hoechst 33342, blue pseudocolor; TFEB red fluorescence, Alexa-568). Ctrl, contrl untreated cells. White masks, computer algorithm-defined cell boundaries (primary objects); pink masks, computer-identified nuclear TFEB based on the average intensity of Alexa-568 fluorescence. Data, means±SEM, n≥3 independent experiments (500 primary objects counted per well; ≥5 wells/sample per each experiment), **p<0.01, ANOVA. (I) HeLa cells were treated as indicated in full medium for 1 h, and LC3 puncta were quantified by HC. White masks, automatically defined cell boundaries (primary objects); green masks, computer-identified LC3 puncta (target objects). Data, means±SEM, n≥3 independent experiments (500 primary objects counted per well; ≥5 wells/sample per each experiment), **p<0.01, ANOVA.

FIG. 2 shows that Ragulator-Rag complex responds to lysosomal damage in control of mTOR. (A) Analysis of mTOR activity in TSC2-deleted (TSC2−/−) and wildtype (TSC2WT) HeLa cells treated with 100 μM GPN in full medium (Full) or starved in EBSS for 1 h. mTOR activity was monitored by immunoblotting analysis of S6K1 (T389) phosphorylation (phosphorylated S6K (p-T389) relative to total S6K). Ctrl, control (untreated cells). (B) Co-immunoprecipitation analysis of changes in interactions between Ragulator and Rag GTPases following treatment with GPN. HEK293T cells stably expressing FLAG-metap2 (control) or FLAG-p14 were treated with 100 μM GPN in full medium for 1 h. Cell lysates were immunoprecipitated (IP) with anti-FLAG antibody and immunoblotted for endogenous RagA or RagC. Data, means±SEM, (n=3), **p<0.01, ANOVA. (C) Immunoprecipitation analysis of interactions between RagA and mTOR/Raptor in cells treated with GPN. HEK293T cells overexpressing HA vector or HA-RagA were treated with 100 μM GPN in full medium for 1 h. Cell lysates were immunoprecipitated with anti-HA antibody and immunoblotted for endogenous mTOR or Raptor. Data, means±SEM, (n=3), *p<0.05, ANOVA. (D) Analysis of mTOR activity in HEK293T cells or HEK293T cells stably expressing constitutively active RagB GTPase (RagBQ99L) treated with 100 μM GPN in full medium or starved in EBSS for 1 h. mTOR activity was monitored as in A. Data, means±SEM, (n=3), † not significant, **p<0.01, ANOVA. (E) Immunofluorescence confocal microscopy visualization of mTOR localization relative to LAMP2-positive lysosomes. Wildtype HEK293T and HEK293T cells stably expressing constitutively active RagB GTPase (RagBQ99L) cells were treated with 100 μM GPN in full medium for 1 h, followed by immunostaining of endogenous LAMP2 (green florescence, Alexa-488) and mTOR (red florescence, Alexa-568). Scale bar, 1 μm. (F) Quantification by HC of overlaps between mTOR and LAMP2 (representative images shown in FIG. S2C) in HEK293T cells and HEK293T cells stably expressing constitutively active RagB GTPase (RagBQ99L) treated with 100 μM GPN in full medium for 1 h. Data, means±SEM, n≥3 independent experiments (500 primary objects counted per well; ≥5 wells/sample per each experiment); † p≥0.05, *p<0.05, ANOVA.

FIG. 3 shows that Gal8 is in dynamic complexes with mTOR and its regulators and Effectors. (A) Analysis of the puncta formation of galectins in response to GPN. HeLa cells overexpressing YFP-fused with the indicated galectins were treated with 100 μM GPN or without (Ctrl) in full medium for 1 h and galectin puncta were quantified by HC. Figure on the left shows representative images of galectins 1, 3, 8, and 9. White masks, algorithm defined cell boundaries (primary objects); green masks, computer-identified galectins puncta (target objects). Data, means±SEM, n≥3 independent experiments (500 primary objects counted per well; ≥5 wells/sample per each experiment); *p<0.05, **p<0.01, † p≥0.05, ANOVA. (B) Immunoprecipitation analysis of the interactions between galectins and mTOR or RagA GTPase. HEK293T cells overexpressing FLAG-tagged galectins were subjected to anti-FLAG immunoprecipitation followed by immunoblotting for endogenous mTOR or RagA. (C) Immunoprecipitation analysis of interactions between Gal8 and mTOR protein complexes in response to GPN treatment. THP-1 cells not treated or treated with 100 μM GPN in full medium for 1 h were subjected to immunoprecipitation with anti-Gal8 antibody, followed by immunoblotting for endogenous RagA, p14, mTOR or Raptor. (D) Analysis of the proximity of Gal8 to mTOR and other proteins in mTOR complexes in response to GPN. Biotinylated proteins from HEK293T cell expressing APEX2-vector or APEX2-Gal8, after GPN and biotin phenol (BP) treatment were affinity-enriched by binding to streptavidin-beads, and samples were analyzed by immunoblotting analysis for endogenous RagA, p14, mTOR or Raptor. Data, means±SEM, (n=3), **p<0.01, ANOVA. (E) Immunoprecipitation analysis of the interactions between Gal8 and RagA GTPase and its mutants. Lysates of HEK293T cells overexpressing FLAG-Gal8 and HA-tagged RagA proteins (RagA_{WT}, RagA_{T21L} or RagA_{Q66L}) were subjected to anti-FLAG immunoprecipitation, followed by immunoblotting for HA-tagged RagA proteins. (F) Immunoprecipitation analysis of the interactions between Gal8 and RagC GTPase and its mutants. HEK293T cells overexpressing FLAG-Gal8 and HA-tagged RagC proteins (RagC_{WT}, RagC_{S75L} or RagC_{Q120L}) lysates were subjected to anti-FLAG immunoprecipitation, followed by immunoblotting for HA-tagged RagC proteins.

FIG. 4. Gal8 is required for mTOR inactivation in response to lysosomal damage. (A) Analysis of mTOR activity in parental HeLa (Gal8WTHeLa) and Gal8-knockout (Gal8KOHeLa) HeLa cells treated with 100 μM GPN in full medium for 1 h. mTOR activity was monitored by immunoblotting analysis of S6K1 (T389) and ULK1 (S757) phosphorylation (phosphorlated S6K p-T389 and ULK1 p-S757 relative to total S6K and ULK1, respectively). Data, means±SEM, (n=3), † p≥0.05, **p<0.01, ANOVA. (B) Analysis of autophagy induction in Gal8WTHeLa and Gal8KOHeLa HeLa cells treated with 100 μM GPN in full medium for 1 h. Autophagy induction was monitored by immunoblotting analysis of lapidated LC3 (LC3-II). Data, means±SEM (n=3), **p<0.01, ANOVA. (C) Analysis of mTOR activity in bone marrow-derived macrophages (BMMs). BMMs of wild type C57BL (Gal8WTBMM) and their littermate Gal8-knockout mice (Gal8KOBMM) were treated with 400 μM GPN in full medium for 1 h. The mTOR activity was monitored as in A. Data, means±SEM (n=3), † p≥0.05, **p<0.01, ANOVA. (D) Analysis of autophagy induction in Gal8WTBMM and Gal8KOBMM treated with 400 μM GPN in full medium for 1 h. Autophagy induction was monitored as in B. Data, means±SEM (n=3), **p<0.01, ANOVA. (E) HC analysis of TFEB nuclear translocation in Gal8WTBMM and Gal8KOBMM treated with 400 μM GPN in full medium for 1 h. White masks, algorithm-defined cell boundaries (primary objects); pink masks, computer-identified nuclear TFEB based on the average intensity. Data, means±SEM, n≥3 independent experiments (500 primary objects counted per well; 25 wells/sample per each experiment), **p<0.01, ANOVA.

FIG. 5 shows that lysosomal damage promotes interactions between Gal8 and the amino acid and cholesterol sensor SLC38A9. (A) Analysis of interactions between Gal8 and SLC38A9 in response to GPN. HEK293T cells overexpressing FLAG-SLC38A9 were treated with 100 μM GPN in full medium or starved in EBSS for 1 h. Cell lysates were subjected to anti-FLAG immunoprecipitation and immunoblotted for endogenous Gal8. Control (Ctrl), untreated cells. Note: SLC38A9 is known as a heavily glycosylated protein giving a smear pattern in immunoblots. (B) Immunoprecipitation analysis of the interactions between SLC38A9 and Gal8 mutated for the glycan recognition sites. HEK293T cells overexpressing GFP-tagged Gal8 or glycan recognition mutant forms of Gal8 (individual R69H, R232H or double/combined R69H & R232H; see panel C) were treated with 100 μM GPN for 1 h in full medium. Cell lysates were subjected to immunoprecipitation with anti-SLC38A9 antibody, followed by immunoblotting for GFP-tagged Gal8 proteins. *, non-specific bands. Note: input SLC3A89 was deglycosylated with PNGase F. (C) Schematic diagram of Gal8 domains (CRD and CRD2, carbohydrate recognition domains 1 and 2) and summary of interactions analysis between SLC38A9 and Gal8. +++, strong interaction; +, weak interaction; −, no detected interaction.

FIG. 6 shows that SLC38A9 is required for mTOR reactivation during recovery from lysosomal damage. (A) Analysis of mTOR activity and autophagy induction in HEK293T cells (WT, wild type) and SLC38A9 knockout (SLC38A9 KO) HEK293T derivatives treated with 100 μM GPN in full medium for the indicated time points. mTOR activity was monitored by immunoblotting analysis of S6K1 phosphorylation at T389 (p-T389). Autophagy induction was monitored by immunoblotting analysis of LC3-II. (B) Analysis of autophagy induction in SLC38A9 KO cells treated with GPN. WT and SLC38A9-KO (SLCKO) HEK293T cells were treated with 100 μM GPN in full medium for 30 min, and LC3 puncta were quantified by HC. White masks, algorithm-defined cell boundaries (primary objects); green masks, computer-identified LC3 puncta (target objects). Data, means±SEM, n≥3 independent experiments (500 primary objects counted per well; ≥ 5 wells/sample per each experiment), **p<0.01, ANOVA. (C) Analysis of mTOR activity recovery and autophagy inhibition in SLC38A9 KO cells after GPN washout. WT and SLC38A9 KO HEK293T cells were treated with 100 μM GPN for 1 h followed by 1 h washout in full medium. mTOR activity was monitored by immunoblotting analysis of S6K1 p-T389 and ULK1 p-S757 phosphorylation. Autophagy induction was monitored by immunoblotting analysis of LC3-II. (D) Analysis of mTOR activity and autophagy induction (as in A) in HEK293T cells transiently transfected with and overexpressing FLAG-SLC38A9 or FLAG (vector control) treated with 100 μM GPN in full medium for indicated time points. (E) HC analysis of autophagy induction in SLC38A9-overexpressing cells treated with GPN (as in D). Control and FLAG-SLC38A9 overexpressing HEK293T cells were treated with 100 μM GPN in full medium for 30 min, and LC3 puncta were quantified by HC. White masks, algorithm-defined cell boundaries (primary objects); green masks, computer-identified LC3 puncta (target objects). Data, means±SEM, n≥3 independent experiments (500 primary objects counted per well; ≥5 wells/sample per each experiment), **p<0.01, ANOVA.

FIG. 7 shows that Galectin 9 interacts with AMPK and activates it during lysosomal damage. (A) Immunoprecipitation analysis of the interactions between galectins and AMPKα. HEK293T cells overexpressing FLAG-tagged galectins were subjected to anti-FLAG immunoprecipitation followed by immunoblotting for endogenous AMPKα. (B) Analysis of the activation of AMPK in parental (Ctrl) and Gal9-knockout (Gal9KO) HEK293A cells treated with 100 μM GPN in full medium for 1. AMPK activation was monitored by immunoblotting analysis of phosphorylated AMPKα (p-T172) and its targets acetyl-CoA carboxylase (ACC, p-S79) and ULK1 (p-S317; vs. p-S757 phosphorylated by mTOR) relative to total AMPKα, ACC and ULK1. (C) Immunoprecipitation analysis of the interactions between endogenous Gal9 and TAK1, LKB1 or CaMKK2 in THP-1 cells. (D) Analysis of the proximity of Gal9 to AMPKα and its upstream regulators. Biotinylated proteins from HEK293T cell lysates generated from APEX2-vector or APEX2-Gal9 after biotin phenol (BP) treatment were isolated by streptavidin chromatography and the samples were analyzed for endogenous TAK1, LKB1 and CaMKK2. (E) HC analysis of autophagy induction (LC3 puncta) in parental (Gal9WT293A) and Gal9-knockout (Gal9KO293A) HEK293A cells treated with 100 μM GPN in full medium for 1 h. White masks, algorithm-defined cell boundaries (primary objects); green masks, computer-identified LC3 puncta (target objects). Data, means±SEM, n≥3 independent experiments (500 primary objects counted per well; ≥5 wells/sample per each experiment), *p<0.01, ANOVA. (F) Analysis of autophagy induction in Gal8WTBMM and Gal8KOBMM primary macrophages treated with 400 μM GPN in full medium for 1 h. LC3 puncta were quantified by HC. White masks, algorithm-defined cell boundaries (primary objects); green masks, computer-identified LC3 puncta (target objects). Data, means±SEM, n≥3 independent experiments (500 primary objects counted per well; ≥5 wells/sample per each experiment), **p<0.01, ANOVA. (G) Survival curves of C57BL/6 mice and their Gal8-knockout littermates in a model of respiratory infection with M. tuberculosis. Initial lung deposition, 700 CFU of M. tuberculosis Erdman.

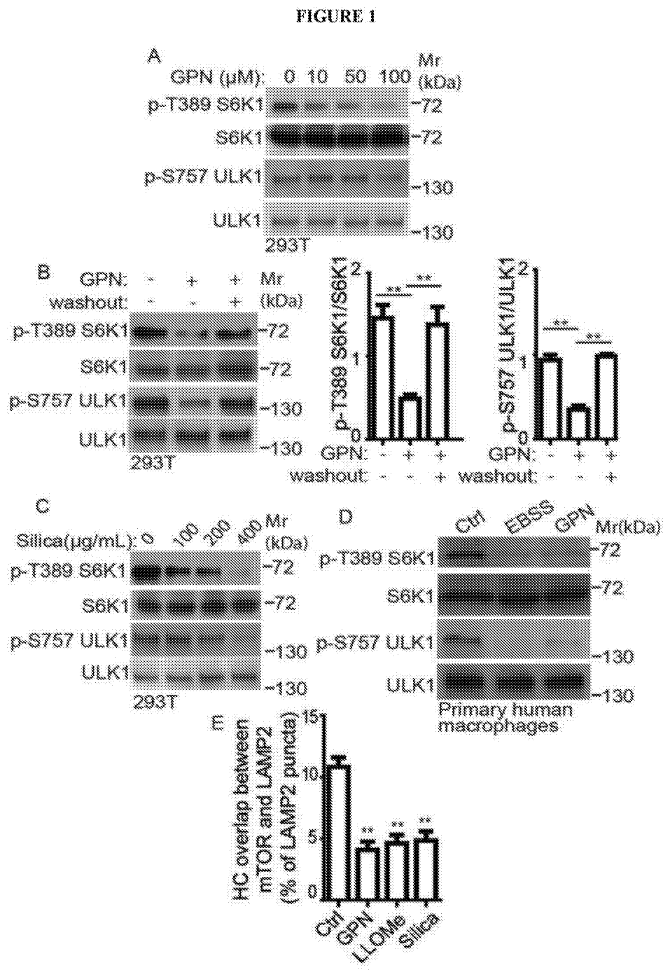

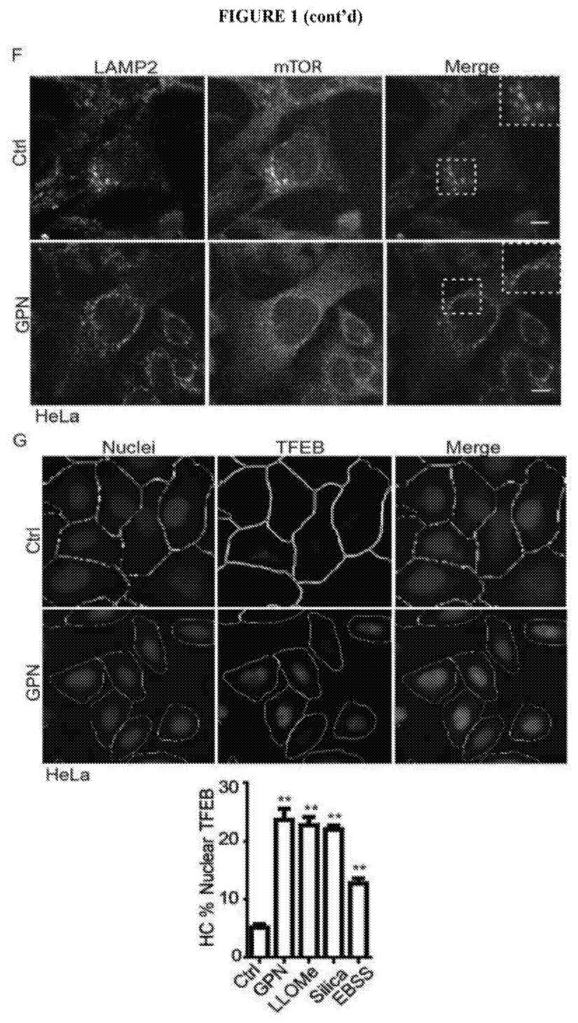

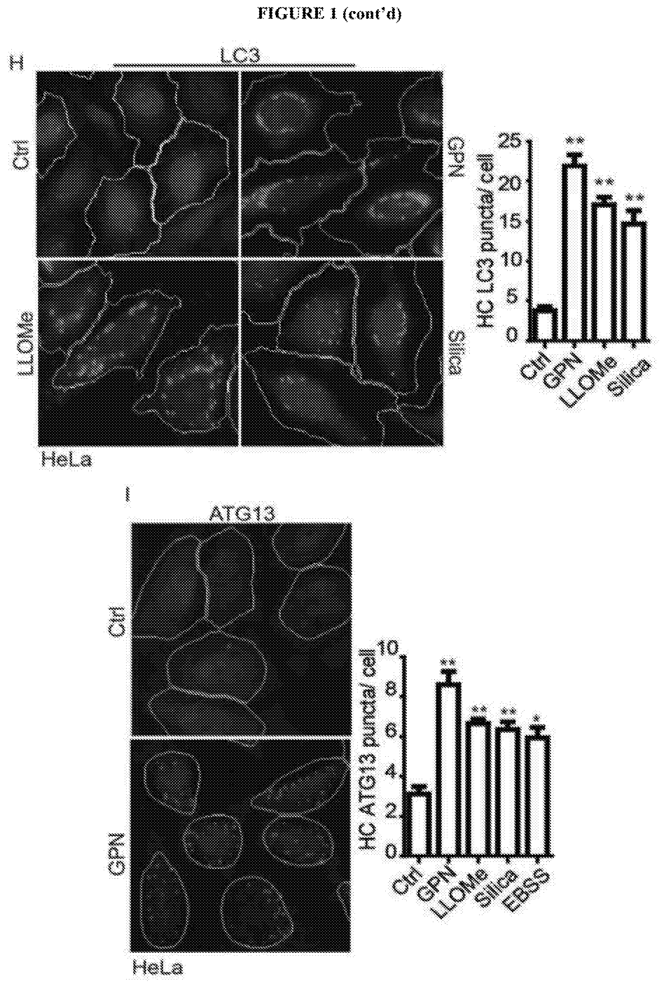

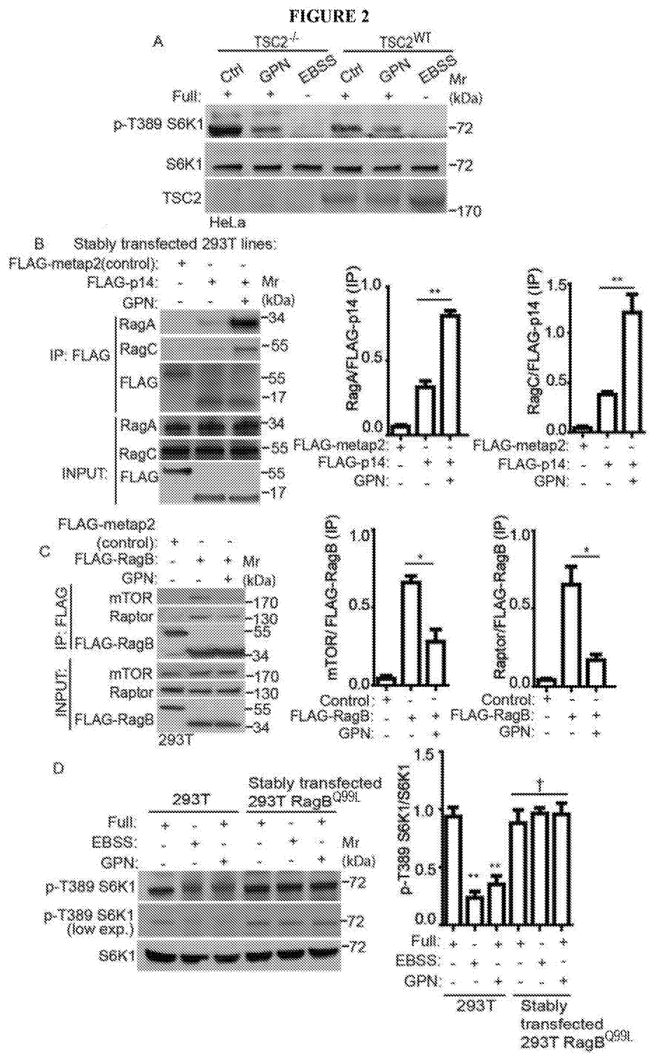

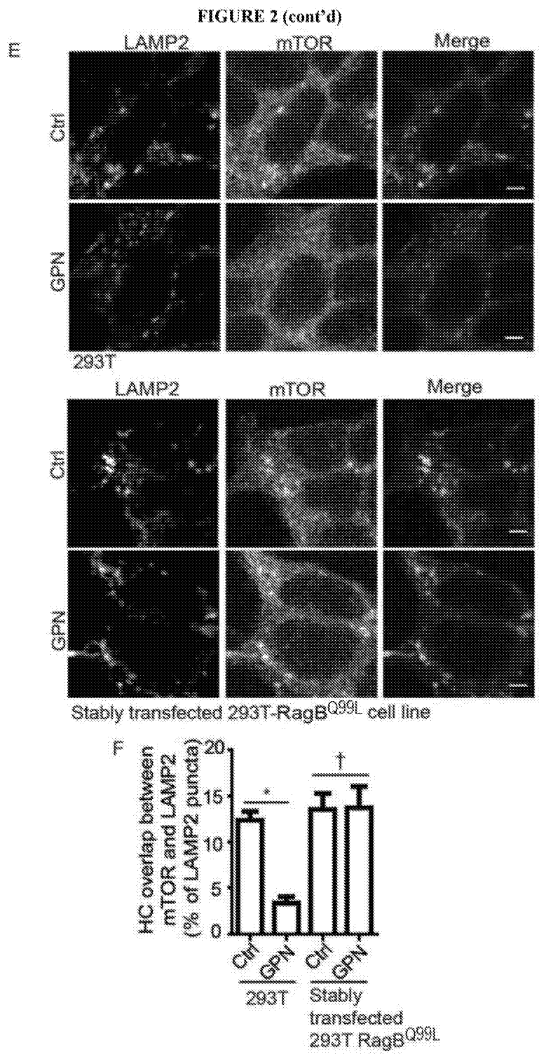

FIG. S1, related to FIG. 1. Lysosomal damage inhibits mTOR signaling.

(A) Analysis of mTOR activity in HeLa cells treated with 100 μM glycyl-L-phenylalanine 2-naphththylamide (GPN) in full medium or starved in EBSS for 1 h. mTOR activity was monitored by immunoblotting analysis of S6K1 (p-T389) and ULK1 (p-S757) phosphorylation (phosporylated S6K (T389) and ULK1 (S757) relative to total S6K and ULK1, respectively). Control (Ctrl), untreated cells. (B) Dose-response analysis of mTOR activity in HEK293T cells treated with Leu-Leu-OMe (LLOMe) as indicated in full medium for 1 h. mTOR activity was monitored by immunoblotting as in A. (C) HEK293T cells were treated with lysosomal damaging agents (100 μM GPN; 2 mM LLOMe; 400 μg/mL Silica) for 1 h in full medium and status of acidified organelles assessed by quantifying LysoTracker Red DND-99 puncta using automated high-content imaging and analysis (HC). None-treated cells were as control (Ctrl). White masks, algorithm defined cell boundaries (primary objects); yellow masks, computer-identified LysoTracker Red puncta (target objects). Data, means±SEM. n≥3 independent experiments (500 primary objects counted per well; ≥25 wells/sample per each experiment), **p<0.01, ANOVA. (D) Immunofluorescence confocal microscopy visualization of mTOR localization relative to LAMP2-positive lysosomes. HeLa cells were treated with 2 mM LLOMe or 400 μg/mL Silica in full medium for 1 h, followed by immunostaining of endogenous LAMP2 (green florescence, Alexa-488) and mTOR (red florescence, Alexa-568). Scale bar, 5 μm. (E) Sample images from quantification scans using HC analysis of overlaps between mTOR and LAMP2 (corresponding data in FIG. 1E) in HeLa cells treated with lysosomal damaging agents for 1 h in full medium. Red and green masks, computer-identified mTOR and LAMP2, respectively (target objects). Control (Ctrl), untreated cells. (F) HC analysis of overlaps between mTOR and LAMP2 in HeLa cells treated with 2 mM LLOMe for 1 h followed by 1 h washout (recovery phase) in full medium. Representative images: red and green masks, computer algorithm-identified mTOR and LAMP2 profiles, respectively (target objects). Ctrl, control untreated cells. Data, means±SEM, n≥3 independent experiments (500 primary objects counted per well; ≥5 wells/sample per each experiment), **p<0.01, ANOVA. (G) Representative HC images: TFEB nuclear translocation in HeLa cells treated with 2 mM LLOMe or 400 μg/mL Silica in full medium, or starved in EBSS for 1 h. Quantitative data are in FIG. 1G. Blue: nuclei, Hoechst 33342; Red: anti-TFEB antibody, Alexa-568. White masks, algorithm-defined cell boundaries (primary objects); pink masks, computer-identified nuclear TFEB based on the average intensity of Alexa-568 fluorescence. (H) LC3-II levels in HEK293T cells treated with lysosomal damaging agents (100 μM GPN; 2 mM LLOMe; 400 μg/mL Silica) in full medium for 1 h. Ctrl, control untreated cells. (I) Examples of HC images: ATG13 puncta in HeLa cells treated as indicated in full medium for 1 h (quantitative HC data, in FIG. 1I). White masks, automatically defined cell boundaries (primary objects); red masks, computer-identified ATG13 puncta (target objects). (J) Schematic summary of the findings in FIGS. 1 and S1.

FIG. S2, related to FIG. 2. Ragulator-Rag complex and mTOR signaling in response to lysosomal damage (A) HC analysis of overlaps between p18 or RagC and LAMP2 in HeLa cells treated with 100 μM GPN in full medium for 1 h. Representative images shown in left panels. Red and green masks, computer-identified p18 or RagC and LAMP2, respectively (target objects). Ctrl (control): untreated cells. Data, means±SEM, n≥3 independent experiments (500 primary objects counted per well; ≥5 wells/sample per each experiment), † p≥0.05, ANOVA. (B) HEK293T cells expressing FLAG vector or FLAGp18 were treated with 100 μM GPN in full medium for 1 h, and cell lysates were subjected to immunoprecipitation with anti-FLAG antibody, followed by immunoblotting analysis of endogenous RagA and RagC. Data, intensity ratio (intensity of RagA or RagC bands normalized to intensity of FLAG-p18 bands in immunoprecipitated material). Data, means±SEM, n=3, *p<0.05, ANOVA. (C) Representative images corresponding to quantification by HC in FIG. 2F of overlaps between mTOR and LAMP2 in HEK293T cells and HEK293T cells stably expressing constitutively active RagB GTPase (RagBQ99L) treated with 100 μM GPN in full medium for 1 h. Red and green masks, computer-identified mTOR and LAMP2, respectively (target objects). (D) Immunoprecipitation analysis of interactions between RagA and mTOR/Raptor in cells treated with GPN. HEK293T cells overexpressing FLAG-metap2 (control) or FLAGRagA were treated with 100 μM GPN in full medium for 1 h. Cell lysates were immunoprecipitated with anti-FLAG antibody and immunoblotted for endogenous mTOR or Raptor. (E) Pictorial summary of the results shown in FIGS. 2 and S2.

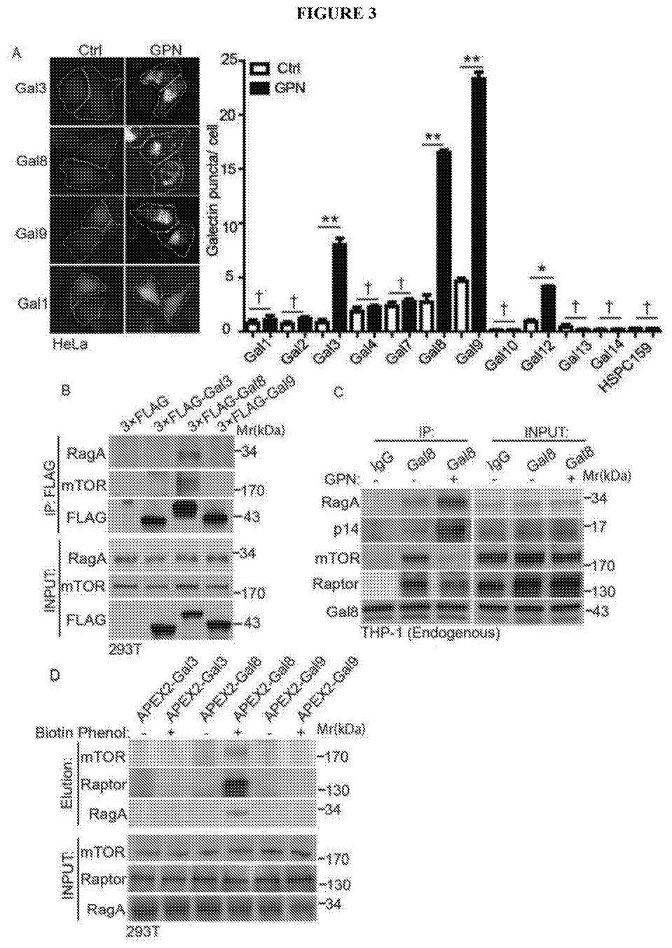

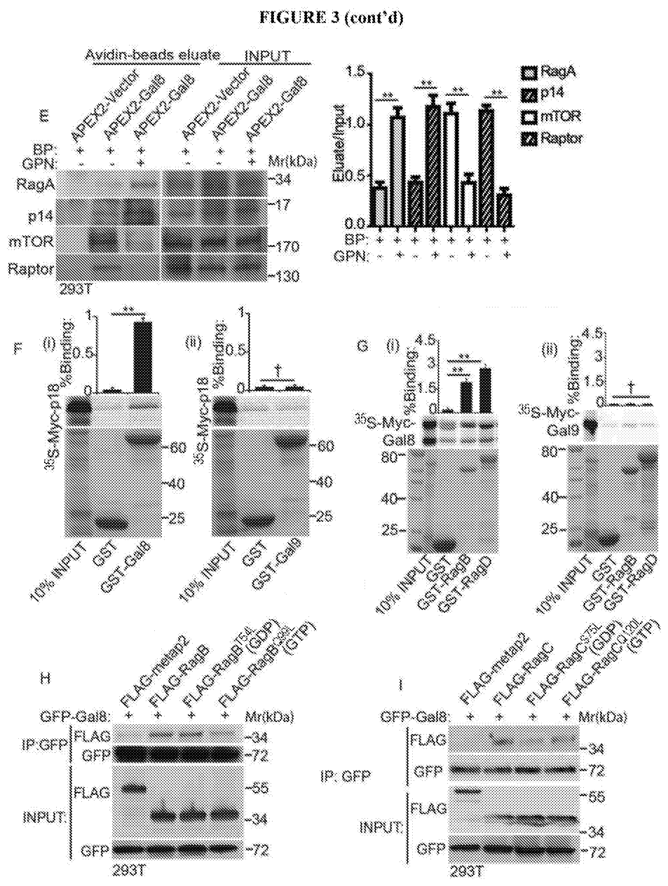

FIG. S3, related to FIG. 3 shows that Gal8 is in dynamic complex with mTOR machinery.

(A) HC analysis of the puncta of galectins in response to chloroquine, Bafilomycin A1 and GPN. HeLa cells overexpressing GFP-Gal8 or GFP-Gal9 were treated with 100 μM chloroquine (CQ), 100 μM Bafilomycin A1 (BAF) or 100 μM GPN in full medium for 1 h, and the puncta of GFP-Gal8 or GFP-Gal9 were quantified by HC. White masks, automatically defined cell boundaries (primary objects); green masks, computer identified GFP-Gal8 or GFP-Ga9 (target objects). Ctrl (control): untreated cells. Data, means±SEM, n≥3 independent experiments (500 primary objects counted per well; ≥5 wells/sample per each experiment), † p≥0.05, **p<0.01, ANOVA. (B) HC analysis of the overlaps between Gal8 and LAMP2 in response to GPN. HeLa cells expressing GFP-Gal8 were treated with 100 μM GPN in full medium for 1 h, and the overlap between GFP-Gal8 and LAMP2 were quantified by HC. White masks, computer algorithm-defined cell boundaries (primary objects); red and green masks, computer identified LAMP2 and GFP-Gal8 puncta (target objects). Ctrl (control): untreated cells. Data, means±SEM, n≥3 independent experiments (500 primary objects counted per well; ≥5 wells/sample per each experiment), **p<0.01, ANOVA.

(C) Immunoprecipitation analysis of the interactions between Gal8 and GTPases. HEK293T cells overexpressing FLAG-tagged Gal8 were immunoprecipitated with anti-FLAG beads. Cell lysates and precipitates were blotted for endogenous RagA, B, C, and D.

(D) Immunoprecipitation analysis of interactions between Gal8 and Ragulator components p14, p18 and MP1. HEK293T cells overexpressing FLAG-tagged Gal8 were immunoprecipitated/collected with anti-FLAG (beads). Cell lysates and collected immune complexes were blotted for endogenous p18, p14, and MP1. (E)(i-ii) GST pulldown assay with in vitro translated Myc-tagged Gal8 or Gal9 and GST-tagged p18. GST-tagged p18 immobilized on Gluthatione sepharose beads were incubated with in vitro translated Myc-tagged Gal8 or Gal9 radiolabeled with 35S-methionine. Interactions were assessed by autoradiography. Data (% binding), means±SEM, n=3, † p≥0.05, **p<0.01, ANOVA. (F)(i-iv) GST pulldown assay with in vitro translated Myc-tagged Rag proteins and GST-tagged Gal8. GST-tagged Gal8 immobilized on Gluthatione sepharose beads were incubated with in vitro translated Myc-tagged Rag proteins radiolabeled with 35S-methionine. Interactions were assessed by autoradiography. Data, Data (% binding), means±SEM, n=3, **p<0.01, ANOVA. (G) Immunoprecipitation analysis of the interactions between Gal8 and RagA GTPase and its mutants. Lysates of HEK293T cells overexpressing GFP-Gal8 and FLAG-tagged metap2 or RagA proteins (RagAWT, RagAT21L or RagAQ66L) were subjected to anti-GFP immunoprecipitation, followed by immunoblotting for FLAG-tagged RagA proteins. (H) Schematic summary of results in FIGS. 3 and S3, and a model depicting predicted GALTOR complex states based on experimental observations: left, intact lysosome with Gal8 in active (darker shade of blue) mTOR-containing complexes on the cytofacial side of the lysosome limiting membrane; right, upon lysosomal membrane damage, Gal8 gains access to exofacially (lumenally) facing glycoconjugates (represented by a trident), with increased Ragulator-Rags interactions whereas inactive (lighter shade of blue) mTOR-raptor complex dissociates from the lysosome.

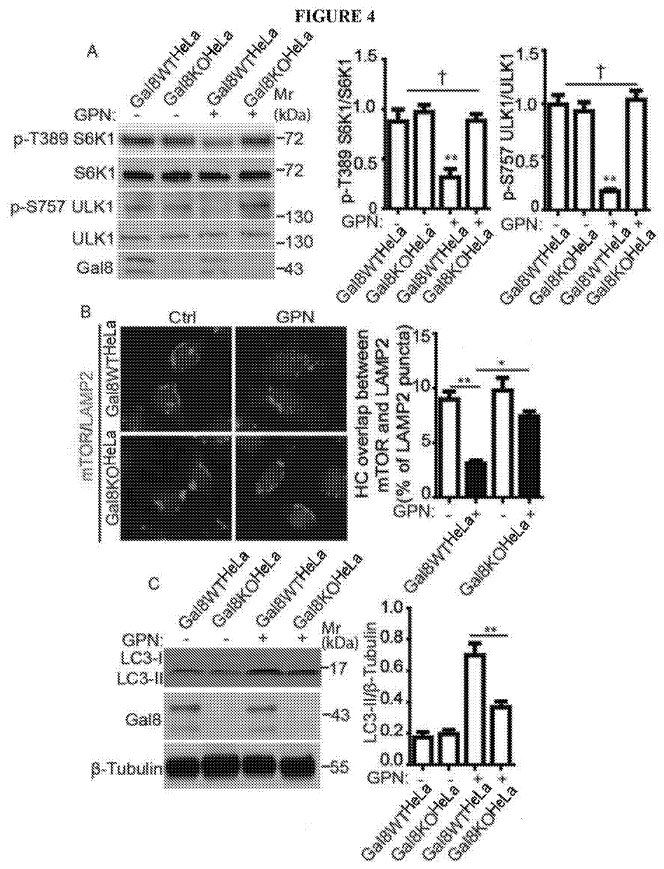

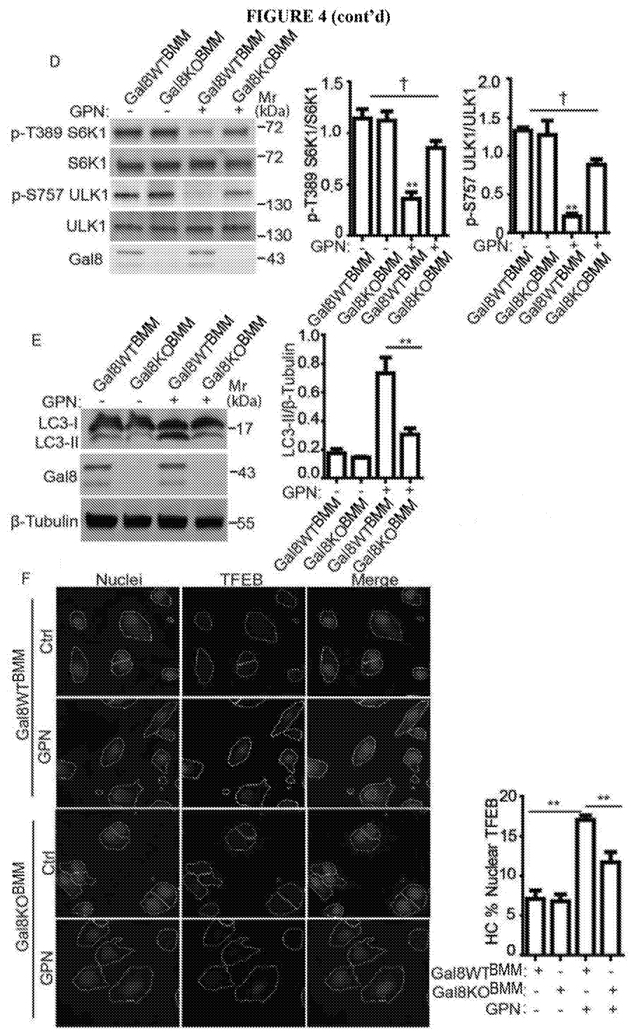

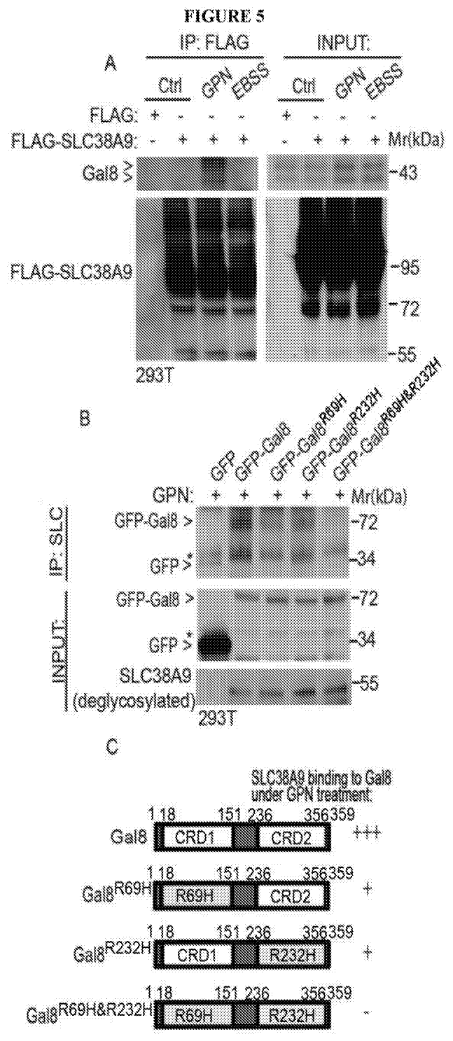

FIG. S4, related to FIG. 4. Gal8 and Gal3 CRISPR knockouts and response to lysosomal damage.

(A) Schematic for CRISPR/Cas9-mediated knockout strategy of LGALS8, and validation of Gal8-knockout (Gal8WTHeLa) by immunoblotting in HeLa cells. (B) Immunoprecipitation analysis of mTOR activity in parental HeLa (Gal8WTHeLa) and Gal8-knockout (Gal8KOHeLa) HeLa cells upon EBSS treatment and glucose starvation (GS)(C) for 1 h. mTOR activity was monitored by immunoblotting analysis of S6K1 (T389) phosphorylation (phosphorylated S6K (T389) relative to total S6K). (D) Immunoprecipitation analysis of mTOR activity in wildtype (WT) and the complementation of Gal8 in Gal-knockout (Gal8KO) HeLa cells upon GPN treatment. Gal8KO HeLa cells overexpressing FLAG-tagged full-length or truncated Gal8 were treated with 100 μM GPN for 1 h in full medium. mTOR activity was monitored by immunoblotting analysis of S6K1 (T389) phosphorylation (phosphorylated S6K (T389) relative to total S6K). (E) Schematic diagram of Gal domains and deletion constructs. (F) HEK293T cells overexpressing GFP-tagged full-length or truncated Gal8 and FLAGRagB or FLAG-p18 were subjected to anti-GFP immunoprecipitation, followed by immunoblotting for FLAG-RagB or FLAG-p18. (G)(i) GST pulldown assay with in vitro translated Myc-tagged Gal8 wildtype or mutants and GST-tagged RagA and RagC. GST-tagged RagA and RagC immobilized on Gluthatione sepharose beads were incubated with in vitro translated Myc-tagged Gal8 wildtype or mutants radiolabeled with 35S-methionine. Interactions were detected by autoradiography. (ii) GST pulldown assay with in vitro translated Myc-tagged Gal8 wildtype or mutants and GST-tagged RagB and RagD. GST-tagged RagB and RagD immobilized on Gluthatione sepharose beads were incubated with in vitro translated Myc-tagged Gal8 wildtype or mutants radiolabeled with 35S-methionine. Interactions were detected by autoradiography. (iii) GST pulldown assay with in vitro translated Myc-tagged Gal8 wildtype or mutants and GST-tagged LAMTOR1/p18. GST-tagged p18 immobilized on Gluthatione sepharose beads were incubated with in vitro translated Myc-tagged Gal8 wildtype or mutants radiolabeled with 35S-methionine. Interactions were detected by autoradiography. CBB: Coomassie brilliant blue staining. (H) Schematic for CRISPR/Cas9-mediated knockout strategy of LGALS3, and validation of Gal3-knockout (Gal3WTHeLa) by immunoblotting in HeLa cells. (I) Analysis of mTOR activity in parental HeLa (Gal3WTHeLa) and Gal3-knockout (Gal3KOHeLa) HeLa cells treated with 100 μM GPN in full medium for 1 h. mTOR activity was monitored by immunoblotting analysis of S6K1 (T389) and ULK1 (S757) phosphorylation (phosphorylated S6K (T389) and ULK1 (S757) relative to total S6K and ULK1, respectively). (J) Primary bone marrow-derived macrophages (BMMs) cells were treated with lysosomal damaging agents for 1 h in full medium and status of acidified organelles assessed by quantifying LysoTracker Red DND-99 puncta using HC. White masks, algorithm-defined cell boundaries (primary objects); red masks, computer identified LysoTracker Red puncta (target objects). Ctrl (control): untreated cells. Data, means±SEM, n≥3 independent experiments (500 primary objects counted per well; ≥5 wells/sample per each experiment), *p<0.05, ANOVA. (K) Schematic summary of the results shown in FIGS. 4 and S4.

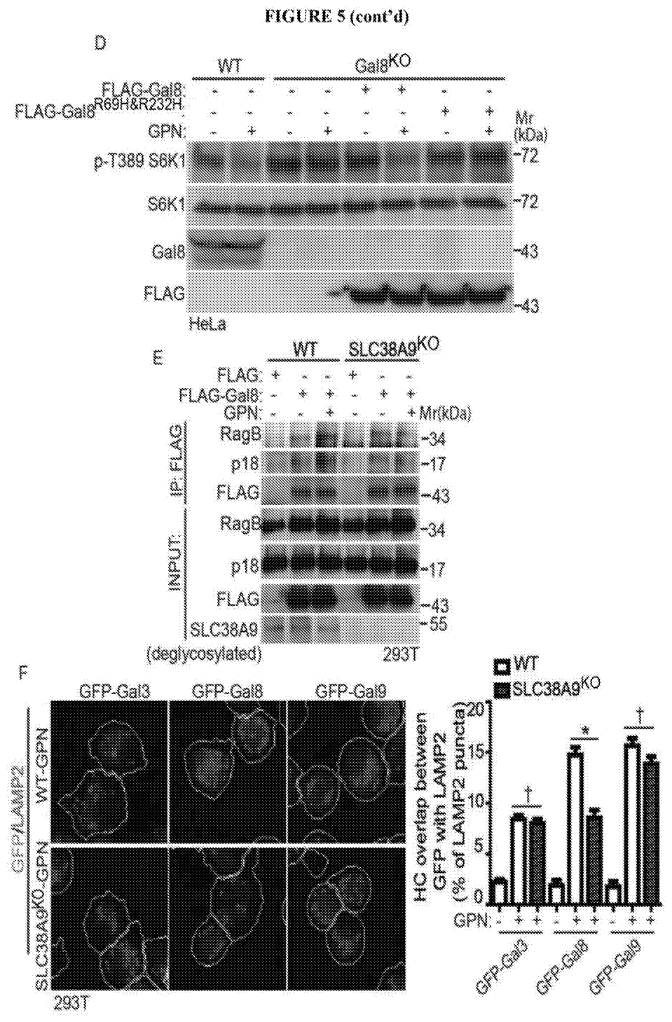

FIG. S5, related to FIGS. 5,6 and 7. Interactions between Gal8 and SLC38A9 and Gal9 CRISPR knockout effects on AMPK.

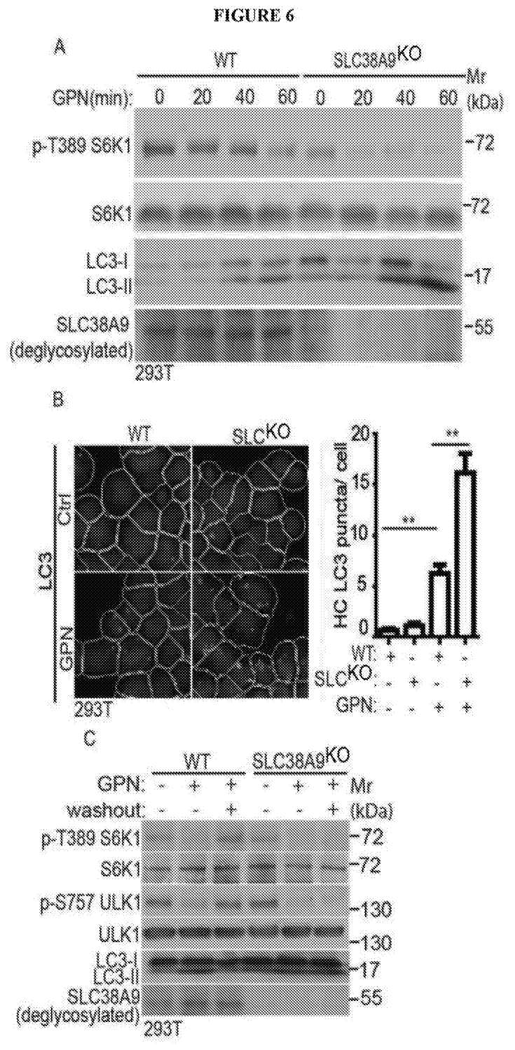

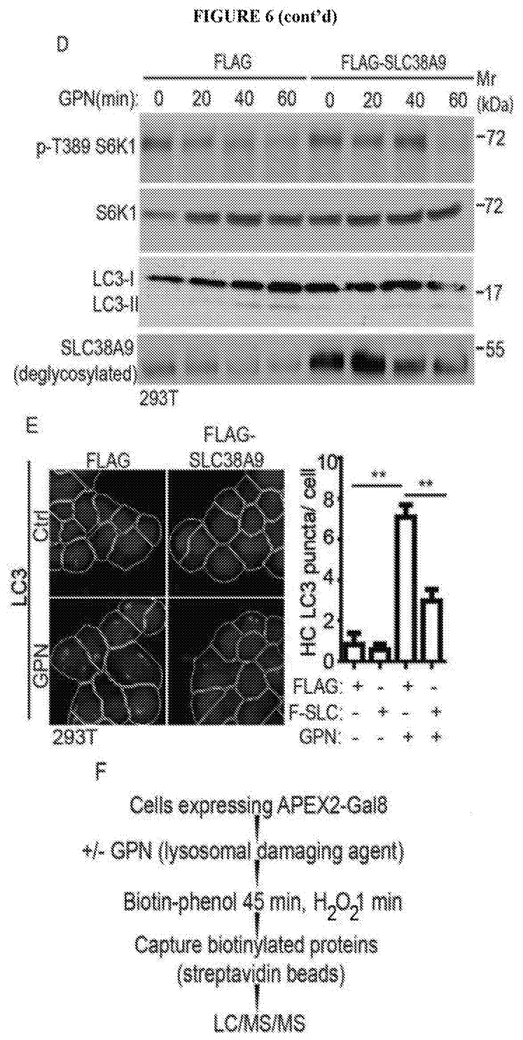

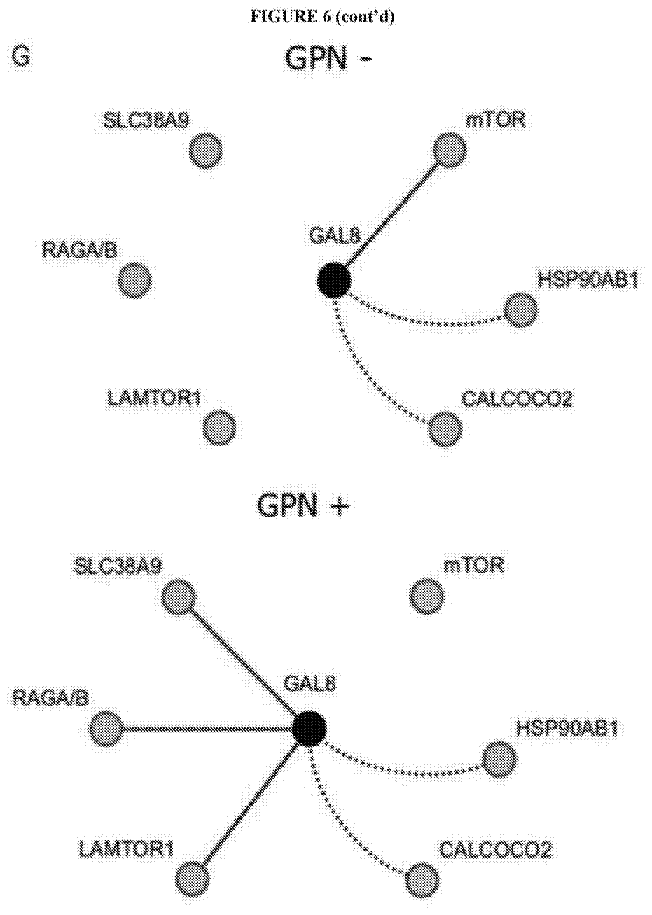

(A) Quantification by HC of overlaps between GFP tagged galectin8 (wild type and glycan recognition-mutant forms) and LAMP2 in HeLa cells expressing corresponding plasmids treated with 100 μM GPN for 1 h in full medium. White masks, automatically defined cell boundaries (primary objects); Red and green masks, computer-identified LAMP2 and GFP tagged proteins, respectively (target objects). None-treated cells were as control (Ctrl). Data, means±SEM, n≥3 independent experiments (500 primary objects counted per well; ≥5 wells/sample per each experiment), † p≥0.05, *p<0.05, **p<0.01, ANOVA. (B) Schematic summary of the results shown in FIG. 5. (C) Schematic summary of the results shown in FIG. 6. (D) Schematic diagram for CRISPR/Cas9-mediated knockout of LGALS9 in HEK293A cells. Gal9-knockout (Gal9KO) was validated by Western blotting. (E) Analysis of the activation of AMPK in wildtype (WT) and Gal9-knockout (Gal9KO) HEK293A cells upon glucose starvation (GS) or 1 μM oligomycin treatment (F) for 1 h. AMPK activity was monitored by immunoblotting analysis of AMPKα (T172) phosphorylation (phosphorylated AMPKα (T172) relative to total AMPKα).

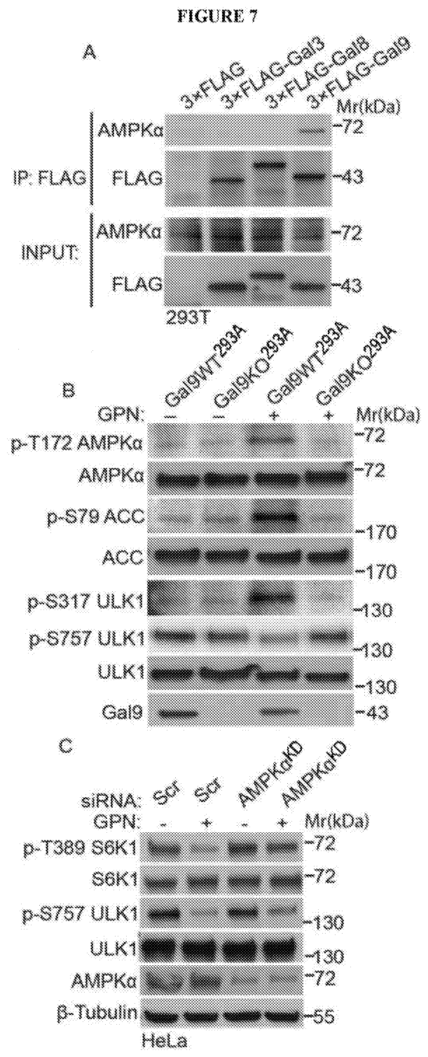

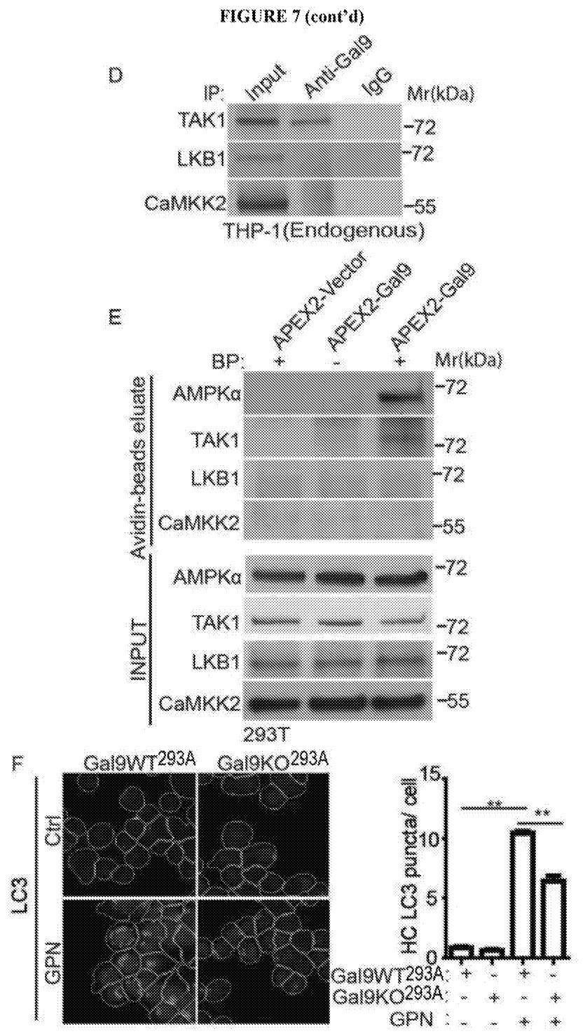

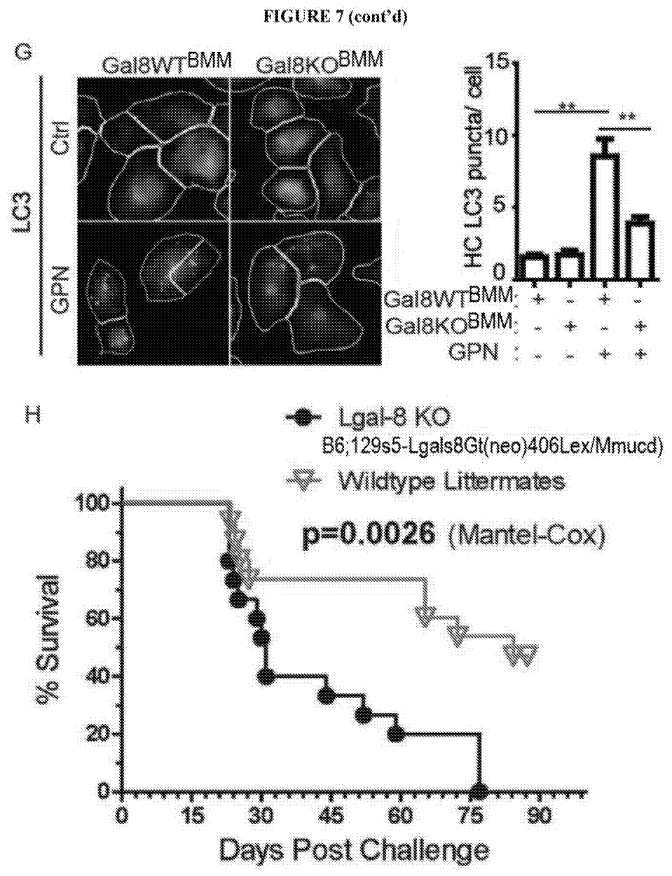

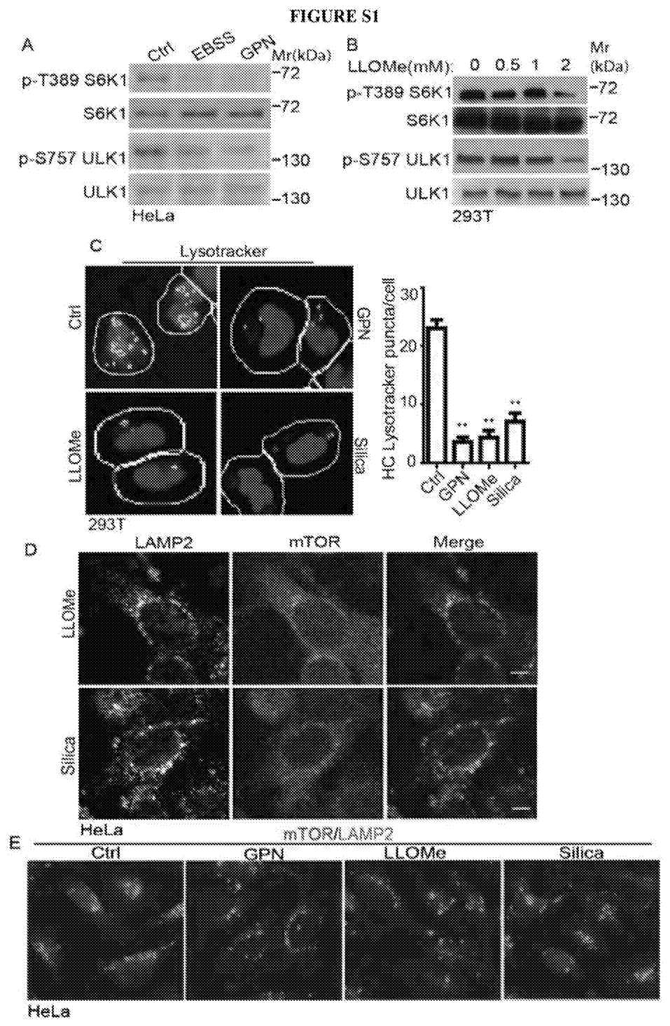

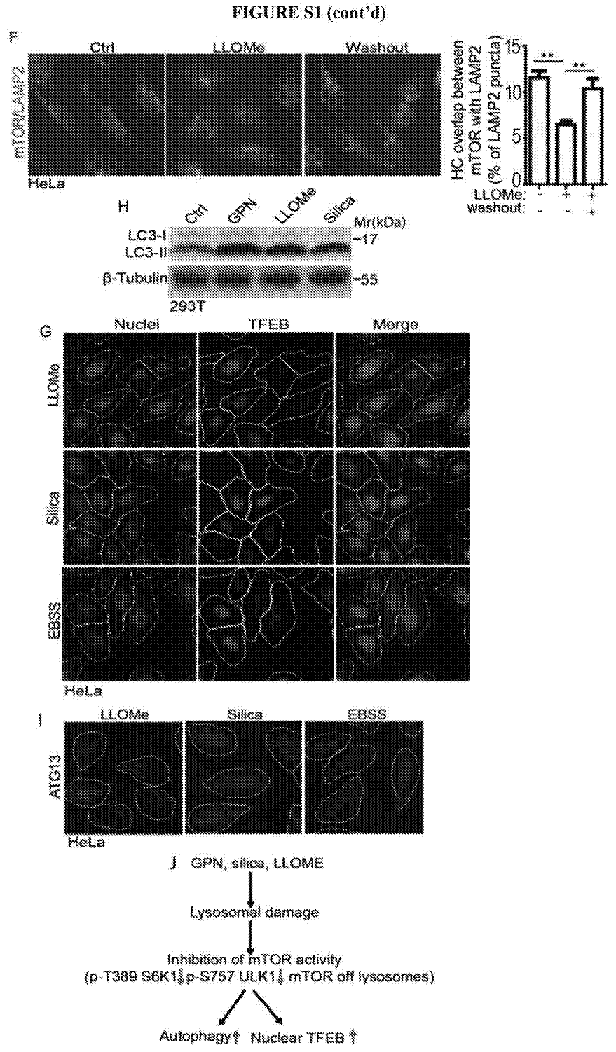

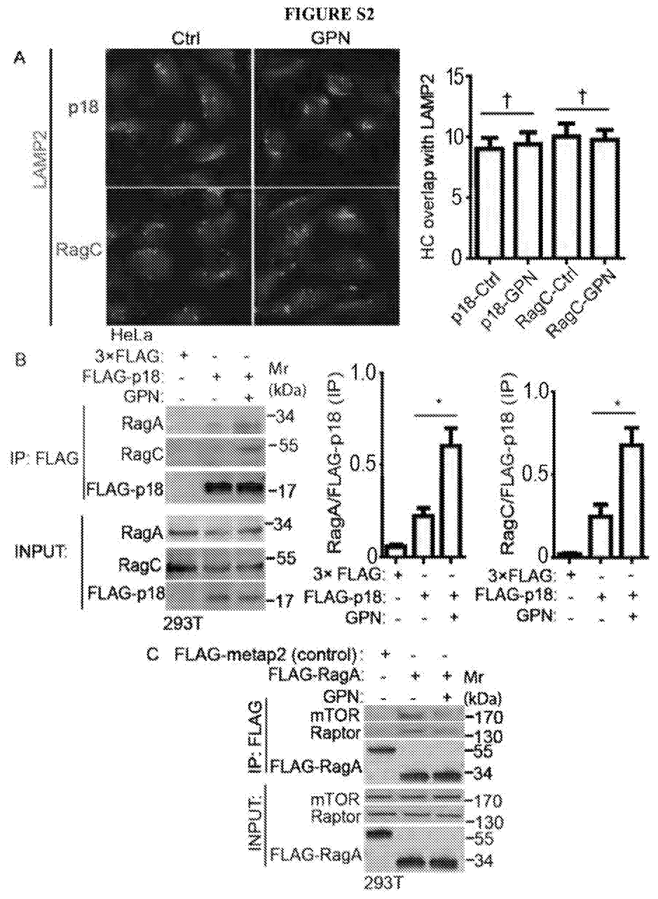

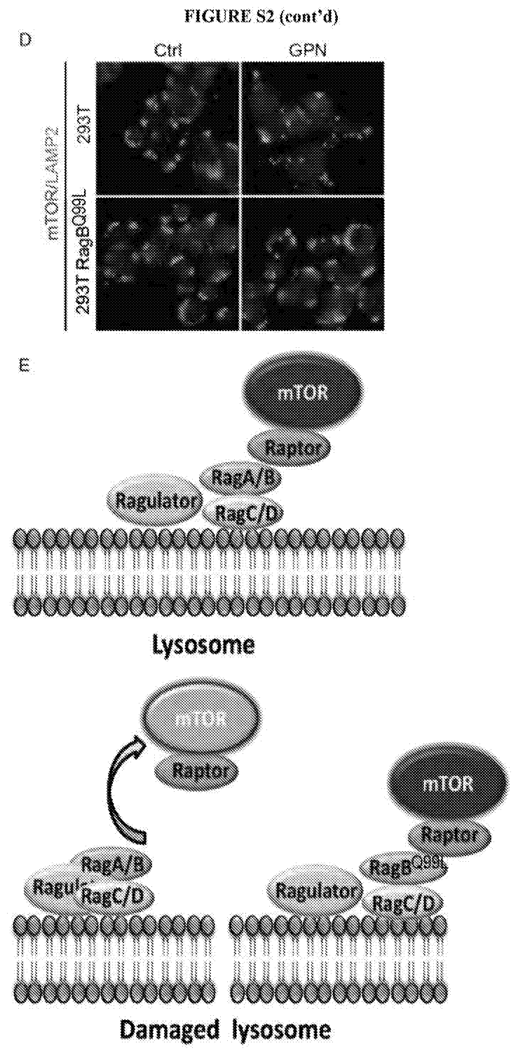

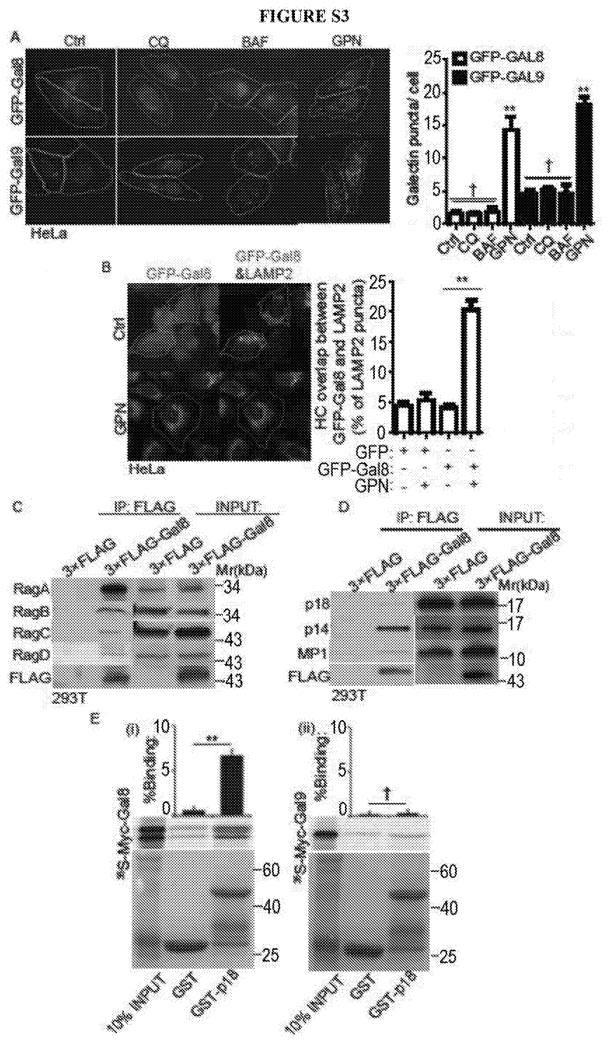

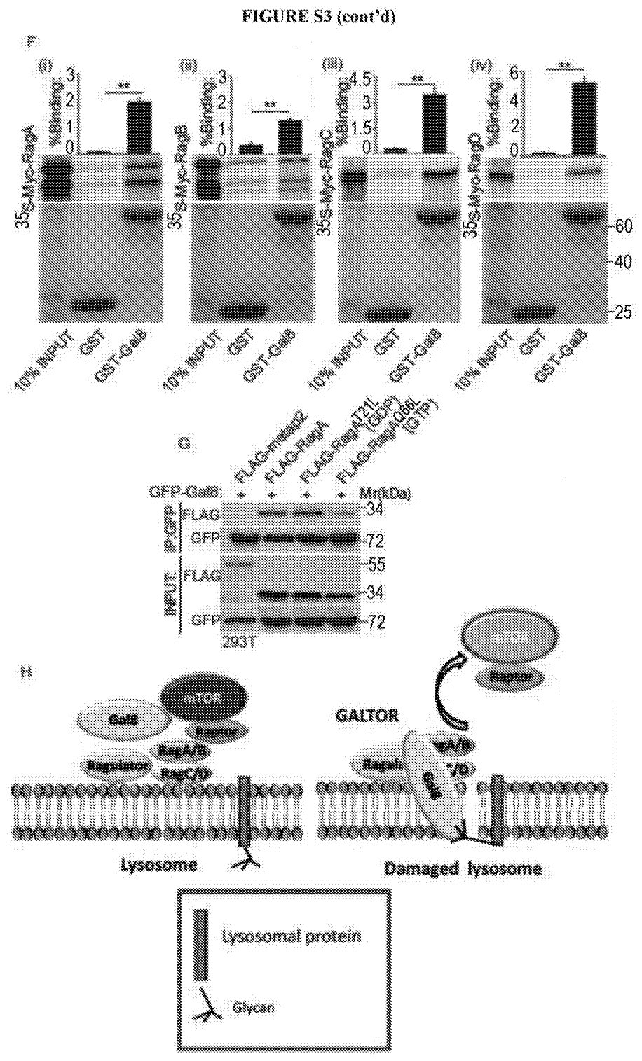

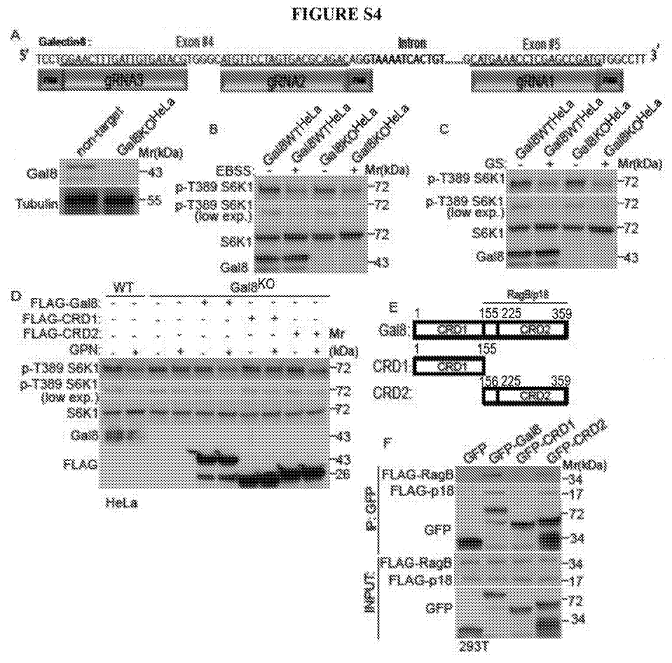

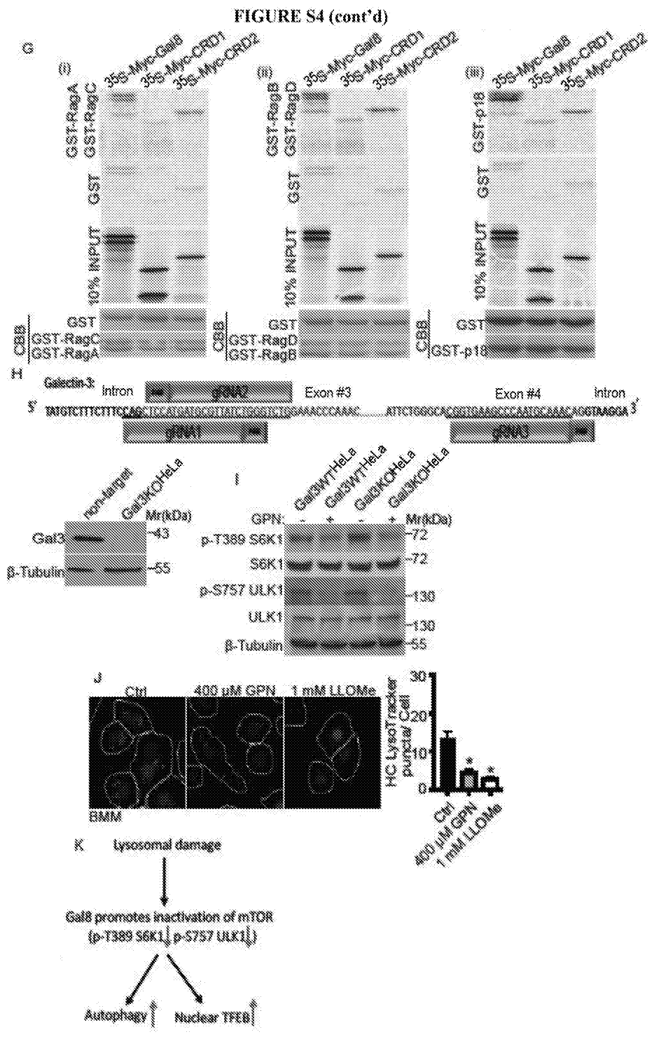

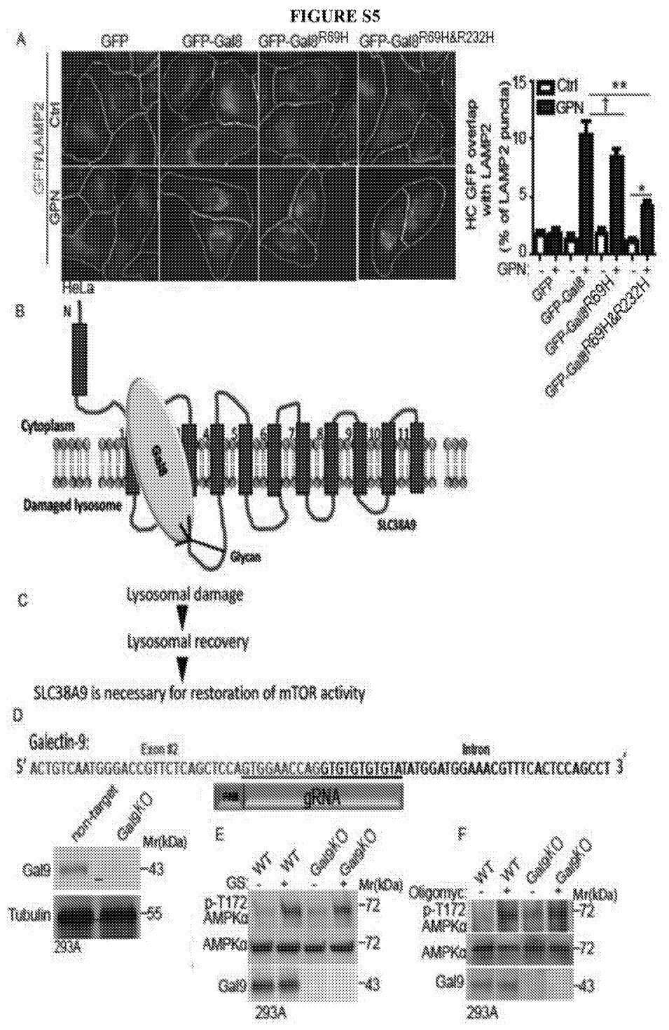

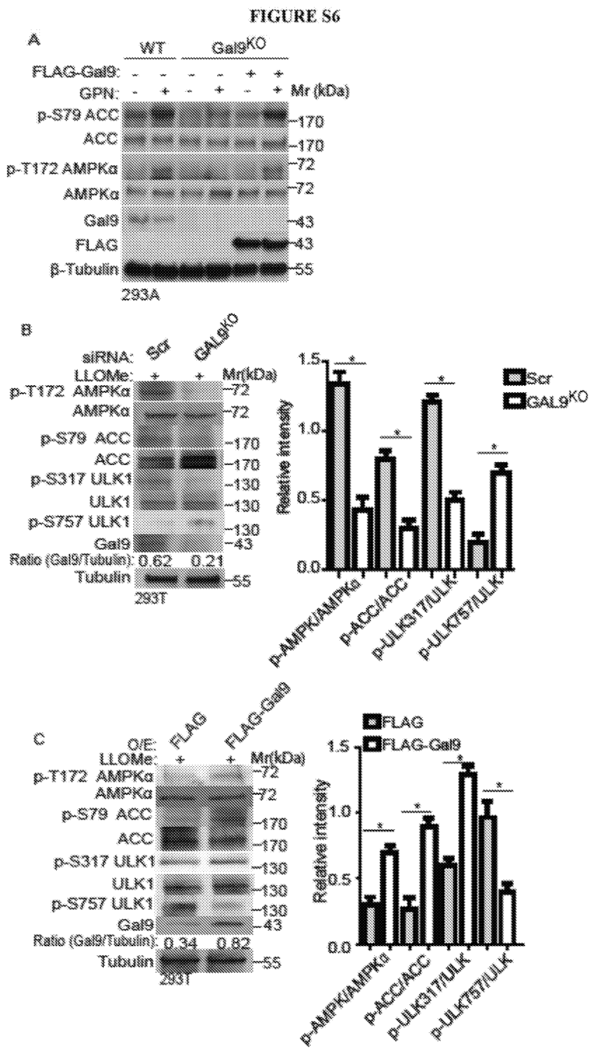

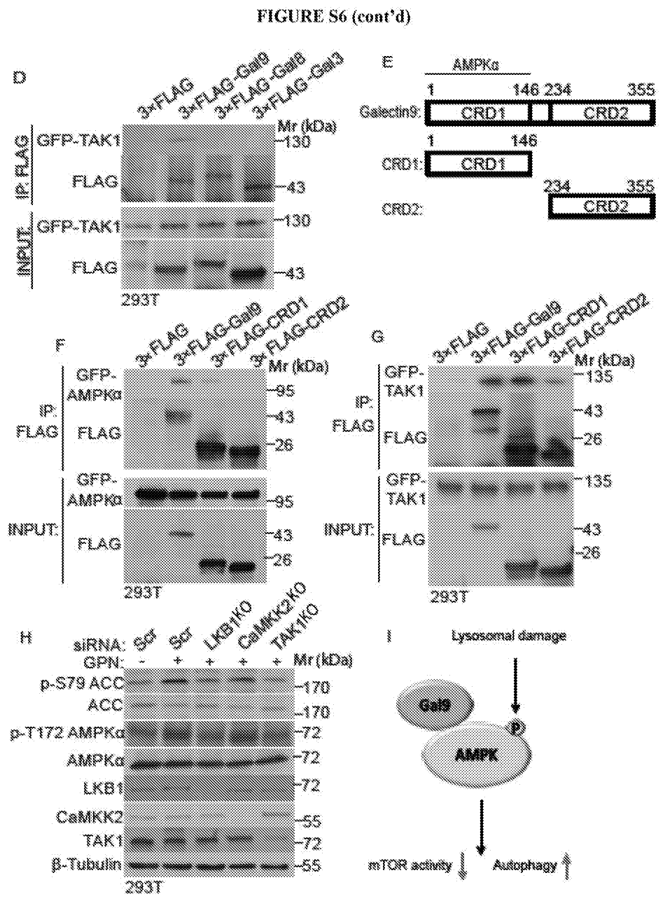

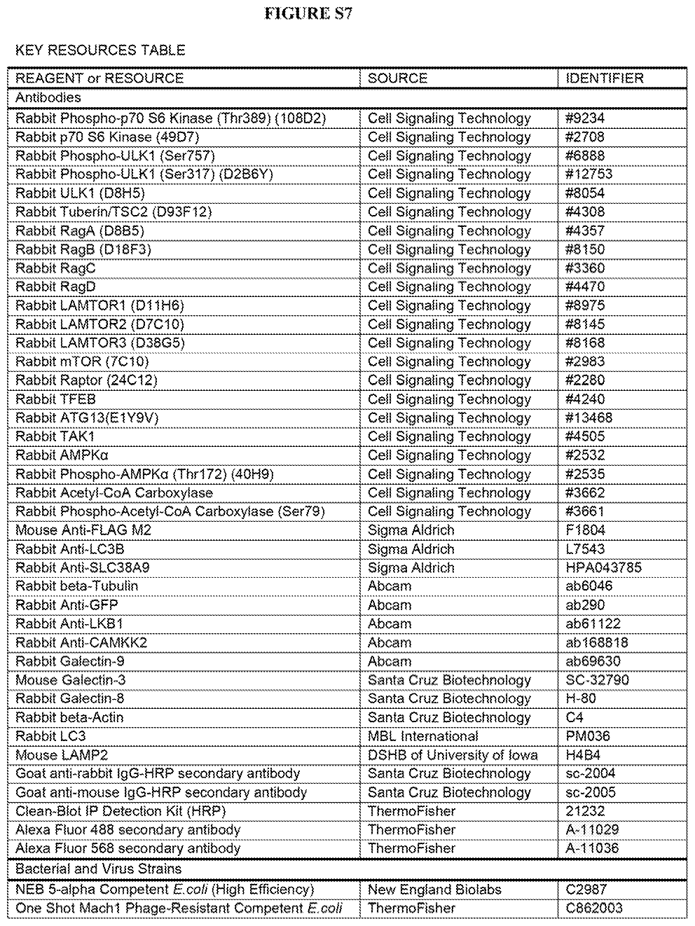

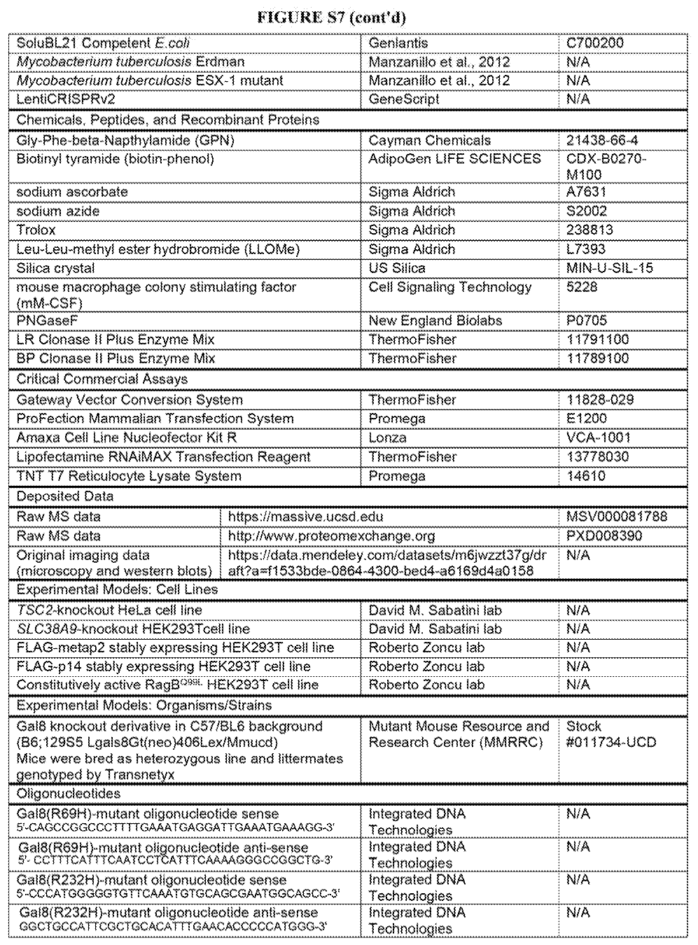

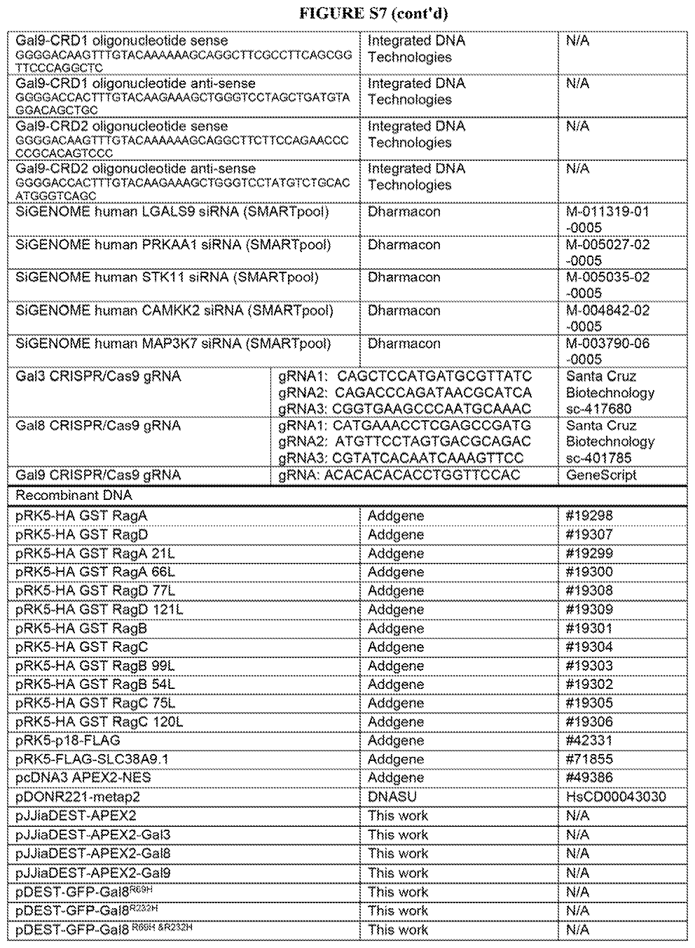

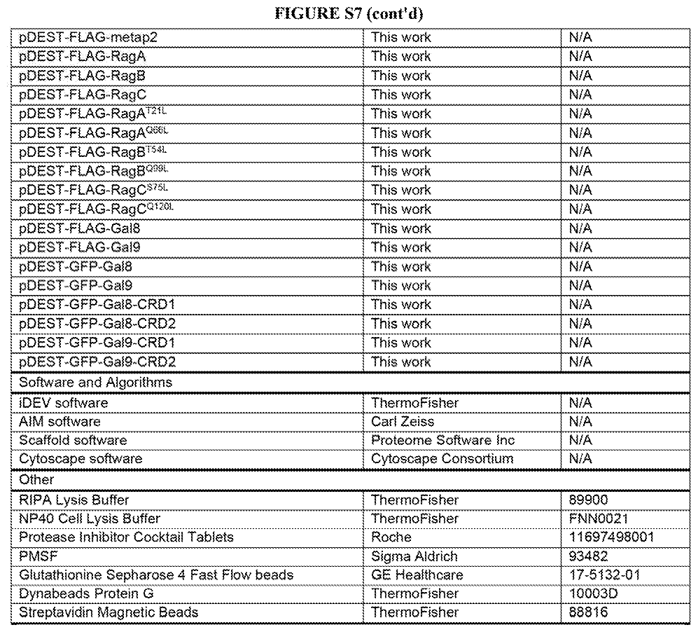

FIG. S6, related to FIG. 7. Analysis of Gal9's role in activation of AMPK in response to lysosomal damage

(A) Analysis of the activation of AMPK in wildtype (WT) and the complementation of Gal9 in Gal9-knockout (Gal9KO) HEK293A cells treated with 100 μM GPN in full medium for 1 h. AMPK activity was monitored by immunoblotting analysis of AMPKα (T172) and acetyl-CoA carboxylase (ACC, S79) phosphorylation (phosphorylated AMPKα (T172) and ACC (S79) relative to total AMPKα and ACC respectively). (B) HEK293T cells transfected with scrambled siRNA (Scr) or Gal9 siRNA (Gal9KD) were treated with 2 mM LLOMe in full medium for 1 h, and the cell lysates were analyzed for phosphorylation of indicated proteins. Data, means±SEM, n=3, *p<0.05, ANOVA. (C) HEK293T cells overexpressing FLAG-Gal9 were treated with 2 mM LLOMe in full medium for 1 h. Cell lysates were analyzed for the indicated proteins. Data, means±SEM, n=3, *p<0.05, ANOVA. (D) Immunoprecipitation analysis of the interactions between galectins and TAK1. HEK293T cells overexpressing FLAG-tagged galectins and GFP-tagged TAK1 were subjected to anti-FLAG immunoprecipitation followed by immunoblotting for GFP-tagged TAK1. (E) Schematic diagram of Gal9 domains and deletion constructs. (F) HEK293T cells overexpressing FLAG-tagged full-length or truncated Gal9 and GFPAMPK were subjected to anti-FLAG immunoprecipitation, followed by immunoblotting for GFP-AMPK. (G) HEK293T cells overexpressing FLAG-tagged full-length or truncated Gal9 and GFP-TAK1 were subjected to anti-FLAG immunoprecipitation, followed by immunoblotting for GFP-TAK1. (H) Analysis of the activation of AMPK in HEK293T cells subjected to knockdowns as indicated treated with 100 μM GPN in full medium for 1 h. AMPK activity was monitored by immunoblotting analysis of AMPKα (T172) and acetyl-CoA carboxylase (ACC, S79) phosphorylation (phosphorylated AMPKα (T172) and ACC (S79) relative to total AMPKα and ACC respectively). Cells transfected with scrambled siRNA were as control (Scr). (1) Schematic summary of the results is shown in FIG. 7A, B.

FIG. S7. Shows the Key Resource Table. A key resource table is provided for the experiments and examples conducted as described herein.

### DETAILED DESCRIPTION OF THE INVENTION

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

The term “compound” or “agent”, as used herein, unless otherwise indicated, refers to any specific chemical compound or composition (such as Galectin-8 or Galectin-9, galactose, another mTOR inhibitor and/or a lysosomotropic agent and/or an autophagy modulator agent) disclosed herein and includes tautomers, regioisomers, geometric isomers as applicable, and also where applicable, stereoisomers, including diastereomers, optical isomers (e.g. enantiomers) thereof, as well as pharmaceutically acceptable salts or alternative salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

The term “patient” or “subject” is used throughout the specification within context to describe an animal, generally a mammal, including a domesticated mammal including a farm animal (dog, cat, horse, cow, pig, sheep, goat, etc.) and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal, often a human.

The terms “effective” or “pharmaceutically effective” are used herein, unless otherwise indicated, to describe an amount of a compound or composition which, in context, is used to produce or affect an intended result, usually the modulation of autophagy within the context of a particular treatment or alternatively, the effect of a bioactive agent which is coadministered with the autophagy modulator (autotoxin) in the treatment of disease.

The terms “treat”, “treating”, and “treatment”, etc., as used herein, refer to any action providing a benefit to a patient at risk for or afflicted by an autophagy mediated disease state or condition as otherwise described herein. The benefit may be in curing the disease state or condition, inhibiting its progression, or ameliorating, lessening or suppressing one or more symptom of an autophagy mediated disease state or condition, especially including excessive inflammation caused by the disease state and/or condition. Treatment, as used herein, encompasses therapeutic treatment and in certain instances, prophylactic treatment (i.e., reducing the likelihood of a disease or condition occurring), depending on the context of the administration of the composition and the disease state, disorder and/or condition to be treated.

As used herein, the term “autophagy mediated disease state or condition” refers to a disease state or condition that results from disruption in autophagy or cellular self-digestion. Autophagy is a cellular pathway involved in protein and organelle degradation, and has a large number of connections to human disease. Autophagic dysfunction which causes disease is associated with metabolic disorders, neurodegeneration, autoimmune diseases, microbial (especially bacterial and viral) infections (especially HIV, HAV, HBV and/or HCV), cancer, aging, cardiovascular diseases and metabolic diseases including diabetes mellitus, among numerous other disease states and/or conditions. Although autophagy plays a principal role as a protective process for the cell, it also plays a role in cell death. Disease states and/or conditions which are mediated through autophagy (which refers to the fact that the disease state or condition may manifest itself as a function of the increase or decrease in autophagy in the patient or subject to be treated and treatment requires administration of an inhibitor or agonist of autophagy in the patient or subject) include, for example, lysosomal storage diseases (discussed hereinbelow), neurodegeneration (including, for example, Alzheimer's disease, Parkinson's disease, Huntington's disease; other ataxias), immune response (T cell maturation, B cell and T cell homeostasis, counters damaging inflammation), autoimmune diseases and chronic inflammatory diseases resulting in excessive inflammation (these disease states may promote excessive cytokines when autophagy is defective), including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (I and II), affecting lipid metabolism islet function and/or structure, excessive autophagy may lead to pancreatic P-cell death and related hyperglycemic disorders, including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high-density lipoprotein (HDL), and elevated triglycerides) and metabolic syndrome, liver disease (excessive autophagic removal of cellular entities-endoplasmic reticulum), renal disease (apoptosis in plaques, glomerular disease), cardiovascular disease (especially including infarction, ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, gout, silicosis, atherosclerosis and associated conditions such as cardiac and neurological both central and peripheral manifestations including stroke, age-associated dementia and sporadic form of Alzheimer's disease, and psychiatric conditions including depression), stroke and spinal cord injury, arteriosclerosis, infectious diseases (microbial infections, removes microbes, provides a protective inflammatory response to microbial products, limits adapation of authophagy of host by microbe for enhancement of microbial growth, regulation of innate immunity) including bacterial, fungal, cellular and viral (including secondary disease states or conditions associated with infectious diseases especially including Mycobacterial infections such as M. tuberculosis, and viral infections such as heptatis A, B and C and HIV I and II), including AIDS, among numerous others.

In addition, an autophagy disease state or condition includes autoimmune diseases such as myocarditis, Anti-glomercular Base Membrane Nephritis, lupus erythematosus, lupus nephritis, autoimmune hepatitis, primary biliary cirrhosis, alopecia areata, autoimmune urticaria, bullous pemphagoid, dermatitis herpetiformis, epidermolysis bullosa acquisita, linear IgA disease (LAD), pemphigus vulgaris, psoriasis, Addison's disease, autoimmune polyendocrine syndrome, II and Ill (APS I, APS II, APS I), autoimmune pancreatitis, type I diabetes, autoimmune thyroiditis, Ord's thyroiditis, Grave's disease, autoimmune oophoritis, Sjogren's syndrome, autoimmune enteropathy, Coeliac disease, Crohn's disease, autoimmune hemolytic anemia, autoimmune lymphoproliferative syndrome, autoimmune neutropenia, autoimmune thrombocytopenic purpura, Cold agglutinin disease, Evans syndrome, pernicious anemia, Adult-onset Still's disease, Felty syndrome, juvenile arthritis, psoriatic arthritis, relapsing polychondritis, rheumatic fever, rheumatoid arthritis, myasthenia gravis, acute disseminated encephalomyelitis (ADEM), balo concentric sclerosis, Guillain-Barr syndrome, Hashimoto's encephalopathy, chronic inflammatory demvelinating polyneuropathy, Lambert-Eaton myasthenic syndrome, multiple sclerosis, autoimmune uveitis, Graves opthalmopathy, Granulomatosis with polyangitis (GPA), Kawasaki's disease, vasculitis and chronic fatigue syndrome, among others.

As used herein, the term “autophagy mediated disease state or condition” refers to a disease state or condition that results from disruption in autophagy or cellular self-digestion. Autophagy is a cellular pathway involved in protein and organelle degradation, and has a large number of connections to human disease. Autophagic dysfunction is associated with cancer, neurodegeneration, microbial infection and ageing, among numerous other disease states and/or conditions. Although autophagy plays a principal role as a protective process for the cell, it also plays a role in cell death. Disease states and/or conditions which are mediated through autophagy (which refers to the fact that the disease state or condition may manifest itself as a function of the increase or decrease in autophagy in the patient or subject to be treated and treatment requires administration of an inhibitor or agonist of autophagy in the patient or subject) include, for example, cancer, including metastasis of cancer, lysosomal storage diseases (discussed hereinbelow), neurodegeneration (including, for example, Alzheimer's disease, Parkinson's disease, Huntington's disease; other ataxias), immune response (T cell maturation, B cell and T cell homeostasis, counters damaging inflammation) and chronic inflammatory diseases (may promote excessive cytokines when autophagy is defective), including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (I and II), affecting lipid metabolism islet function and/or structure, excessive autophagy may lead to pancreatic P-cell death and related hyperglycemic disorders, including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high-density lipoprotein (HDL), and elevated triglycerides) and metabolic syndrome, liver disease (excessive autophagic removal of cellular entities-endoplasmic reticulum), renal disease (apoptosis in plaques, glomerular disease), cardiovascular disease (especially including ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, atherosclerosis and associated conditions such as cardiac and neurological both central and peripheral manifestations including stroke, age-associated dementia and sporadic form of Alzheimer's disease, pre-cancerous states, and psychiatric conditions including depression), stroke and spinal cord injury, arteriosclerosis, infectious diseases (microbial infections, removes microbes, provides a protective inflammatory response to microbial products, limits adapation of authophagy of host by microbe for enhancement of microbial growth, regulation of innate immunity) including bacterial, fungal, cellular and viral (including secondary disease states or conditions associated with infectious diseases), including AIDS and tuberculosis, among others, development (including erythrocyte differentiation), embryogenesis/fertility/infertility (embryo implantation and neonate survival after termination of transplacental supply of nutrients, removal of dead cells during programmed cell death) and ageing (increased autophagy leads to the removal of damaged organelles or aggregated macromolecules to increase health and prolong lire, but increased levels of autophagy in children/young adults may lead to muscle and organ wasting resulting in ageing/progeria).

The term “lysosomal storage disorder” refers to a disease state or condition that results from a defect in lysosomomal storage. These disease states or conditions generally occur when the lysosome malfunctions. Lysosomal storage disorders are caused by lysosomal dysfunction usually as a consequence of deficiency of a single enzyme required for the metabolism of lipids, glycoproteins or mucopolysaccharides. The incidence of lysosomal storage disorder (collectively) occurs at an incidence of about 1:5,000-1:10,000. The lysosome is commonly referred to as the cell's recycling center because it processes unwanted material into substances that the cell can utilize. Lysosomes break down this unwanted matter via high specialized enzymes. Lysosomal disorders generally are triggered when a particular enzyme exists in too small an amount or is missing altogether. When this happens, substances accumulate in the cell. In other words, when the lysosome doesn't function normally, excess products destined for breakdown and recycling are stored in the cell. Lysosomal storage disorders are genetic diseases, but these may be treated using autophagy modulators (autostatins) as described herein. All of these diseases share a common biochemical characteristic, i.e., that all lysosomal disorders originate from an abnormal accumulation of substances inside the lysosome. Lysosomal storage diseases mostly affect children who often die as a consequence at an early stage of life, many within a few months or years of birth. Many other children die of this disease following years of suffering from various symptoms of their particular disorder.

Examples of lysosomal storage diseases include, for example, activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM! Ganliosidosis, including infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs and Wolman disease, among others.

An “inflammation-associated metabolic disorder” includes, but is not limited to, lung diseases, hyperglycemic disorders including diabetes and disorders resulting from insulin resistance, such as Type I and Type II diabetes, as well as severe insulin resistance, hyperinsulinemia, and dyslipidemia or a lipid-related metabolic disorder (e.g. hyperlipidemia (e.g., as expressed by obese subjects), elevated low-density lipoprotein (LDL), depressed high-density lipoprotein (HDL), and elevated triglycerides) and insulin-resistant diabetes, such as Mendenhall's Syndrome, Werner Syndrome, leprechaunism, and lipoatrophic diabetes, renal disorders, such as acute and chronic renal insufficiency, end-stage chronic renal failure, glomerulonephritis, interstitial nephritis, pyelonephritis, glomerulosclerosis, e.g., Kimmelstiel-Wilson in diabetic patients and kidney failure after kidney transplantation, obesity, GH-deficiency, GH resistance, Turner's syndrome, Laron's syndrome, short stature, increased fat mass-to-lean ratios, immunodeficiencies including decreased CD4⁺ T cell counts and decreased immune tolerance or chemotherapy-induced tissue damage, bone marrow transplantation, diseases or insufficiencies of cardiac structure or function such as heart dysfunctions and congestive heart failure, neuronal, neurological, or neuromuscular disorders, e.g., diseases of the central nervous system including Alzheimer's disease, or Parkinson's disease or multiple sclerosis, and diseases of the peripheral nervous system and musculature including peripheral neuropathy, muscular dystrophy, or myotonic dystrophy, and catabolic states, including those associated with wasting caused by any condition, including, e.g., mental health condition (e.g., anorexia nervosa), trauma or wounding or infection such as with a bacterium or human virus such as HIV, wounds, skin disorders, gut structure and function that need restoration, and so forth.

An “inflammation-associated metabolic disorder” also includes a cancer and an “infectious disease” as defined herein, as well as disorders of bone or cartilage growth in children, including short stature, and in children and adults disorders of cartilage and bone in children and adults, including arthritis and osteoporosis. An “inflammation-associated metabolic disorder” includes a combination of two or more of the above disorders (e.g., osteoporosis that is a sequela of a catabolic state). Specific disorders of particular interest targeted for treatment herein are diabetes and obesity, heart dysfunctions, kidney disorders, neurological disorders, bone disorders, whole body growth disorders, and immunological disorders.

In one embodiment, “inflammation-associated metabolic disorder” includes: central obesity, dyslipidemia including particularly hypertriglyceridemia, low HDI cholesterol, small dense LDL particles and postpranial lipemia; glucose intolerance such as impaired fasting glucose; insulin resistance and hypertension, and diabetes. The term “diabetes” is used to describe diabetes mellitus type I or type II. The present invention relates to a method for improving renal function and symptoms, conditions and disease states which occur secondary to impaired renal function in patients or subjects with diabetes as otherwise described herein. It is noted that in diabetes mellitus type I and II, renal function is impaired from collagen deposits, and not from cysts in the other disease states treated by the present invention.

Mycobacterial infections often manifest as diseases such as tuberculosis. Human infections caused by mycobacteria have been widespread since ancient times, and tuberculosis remains a leading cause of death today. Although the incidence of the disease declined, in parallel with advancing standards of living, since the mid-nineteenth century, mycobacterial diseases still constitute a leading cause of morbidity and mortality in countries with limited medical resources. Additionally, mycobacterial diseases can cause overwhelming, disseminated disease in immunocompromised patients. In spite of the efforts of numerous health organizations worldwide, the eradication of mycobacterial diseases has never been achieved, nor is eradication imminent. Nearly one third of the world's population is infected with Mycobacterium tuberculosis complex, commonly referred to as tuberculosis (TB), with approximately 8 million new cases, and two to three million deaths attributable to TB yearly. Tuberculosis (TB) is the cause of the largest number of human deaths attributable to a single etiologic agent (see Dye et al., J. Am. Med. Association, 282, 677-686, (1999); and 2000 WHO/OMS Press Release).

Mycobacteria other than M. tuberculosis are increasingly found in opportunistic infections that plague the AIDS patient. Organisms from the M. avium-intracellular complex (MAC), especially serotypes four and eight, account for 68% of the mycobacterial isolates from AIDS patients. Enormous numbers of MAC are found (up to 10¹⁰ acid-fast bacilli per gram of tissue) and consequently, the prognosis for the infected AIDS patient is poor.

In many countries the only measure for TB control has been vaccination with M. bovis bacille Calmette-Guerin (BCG). The overall vaccine efficacy of BCG against TB, however, is about 50% with extreme variations ranging from 0% to 80% between different field trials. The widespread emergence of multiple drug-resistant M. tuberculosis strains is also a concern.

M. tuberculosis belongs to the group of intracellular bacteria that replicate within the phagosomal vacuoles of resting macrophages, thus protection against TB depends on T cell-mediated immunity. Several studies in mice and humans, however, have shown that Mycobacteria stimulate antigen-specific, major histocompatibility complex (MHC) class II- or class I-restricted CD4 and CD8 T cells, respectively. The important role of MHC class I-restricted CD8 T cells was convincingly demonstrated by the failure of 02-microglobulin) deficient mice to control experimental M. tuberculosis infection.

As used herein, the term “tuberculosis” comprises disease states usually associated with infections caused by mycobacteria species comprising M. tuberculosis complex. The term “tuberculosis” is also associated with mycobacterial infections caused by mycobacteria other than M. tuberculosis. Other mycobacterial species include M. avium-intracellulare, M. kansarii, M. fortuitum, M. chelonae, M. leprae, M. africanum, and M. microti, M. avium paratuberctiosis, M. intracellulare, M. scrofulaceum, M. xenopi, M. marinum, M. tulcerans.

An “infectious disease” includes but is limited to those caused by bacterial, mycological, parasitic, and viral agents. Examples of such infectious agents include the following staphylococcus, streplococcaceae, neisseriaaceae, cocci, enterobacteriaceae, pseudomonadaceae, vibrionaceae, campylobacter, pasteurellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, treponema, borrelia, leptospira, mycoplasma, ureaplasma, rickettsia, chlamydiae, systemic mycoses, opportunistic mycoses, protozoa, nematodes, trematodes, cestodes, adenoviruses, herpesviruses, poxviruses, papovaviruses, hepatitis viruses, orthomyxoviruses, paramyxoviruses, coronaviruses, picornaviruses, reoviruses, togaviruses, flaviviruses, bunyaviridae, rhabdoviruses, human immunodeficiency virus and retroviruses.

In certain embodiments, an “infectious disease” is selected from the group consisting of tuberculosis, leprosy, Crohn's Disease, aquired immunodeficiency syndrome, Lyme disease, cat-scratch disease, Rocky Mountain spotted fever and influenza or a viral infection selected from HIV (I and/or II), hepatitis B virus (HBV) or hepatitis C virus (HCV).

The term “Galectin-8” is used to describe the protein Galectin-8. Galectin 8 is a protein of the galectin family of proteins which is encoded by the gene LGALS8 in humans and with respect to the present invention is involved in the control of mTor in response to endomembrane damage and provides a mechanism and target for the treatment of authorphagy-related diseases. The galectins are beta-galactoside-binding lectins which are expressed in tumor and cancer tissue and exhibit carbohydrate recognition sites which are conserved. The galectins are involved in essential functions such as apoptosis, cell-cell adhesion, cell-matrix interaction, cellular and growth regulation, RNA-splicing, development and cell differentiation, among others. A preferred form of galectin-8 for use in the present invention is human galectin-8, a 317 amino acid polypeptide (Genbank AAF19370, Accession 1008815), or one of its five isoforms: Galectin-8 Isoform a (359 aa) (NP_963839.1; NP_006490.3), Isoform b (317 aa)(NP_963837), Isoform X1 (329 aa) (XP_011542490.1), Isoform X2 (287aa)(XP_016856763.1), Isoform X3 (XP_016856764.1). Pharmaceutically acceptable salts and alternative salt forms of Galectin-8 find use in the present invention.

The term “Galectin-9” is used to describe the protein Galectin-9 which, like Galectin-8, is a beta-galactoside-binding lectin protein of the galectin family of proteins. With respect to the present invention, Galectin-9, like Galectin-8, is involved in the control of mTor in response to endomembrane damage and provides a mechanism and target for the treatment of authorphagy-related diseases. Among other activities, Galectin-9 binds galactosides, has a high affinity for certain oligosaccharides, stimulates bactericidal activity in infected macrophages, enhances cell migration, promotes mesenchymal stromal cells to inhibit T-cell proliferation, increases regulatory T-cells and induces cytotoxic T-cell apoptosis following virus infection, activates ERK1/2 phosphorylation inducing cytokine (IL-6, IL-8, IL-12) and chemokine (CCL2) production in mast and dendritic cells, inhibits degranulation and induces apoptosis of mast cells. Galectin-9 is also involved in the maturation and migration of dendritic cells and inhibits natural killer (NK) cell function, among other functions. Preferred Galectin-9 polypeptides for use in the present invention is human Galectin 9 (355 aa) (Genbank CAB93851.1; Unit ProtKB O00182.2) and its three isoforms: Isoform short (323 aa) (NP_002299.2), Isoform long (355aa)(NP_033665.1) and Isoform 3 (246 aa) (NP_001317092.1). Pharmaceutically acceptable salts and alternative salt forms of Galectin-9 find use in the present invention.

Compositions according to the present invention comprise Galectin-8 and/or 9, a Galectin-8 upregulator, a Galectin-9 upregulator, including galactose, a galactose containing sugar or other sugar compound (especially lactose, including N-linked and O-linked lactose such as N-acetyl lactosamine which acts as an agonist or an inhibitor such as a galactoside inhibitor or alternatively, a lactulose amine such as N-lactulose-octamethylenediamine (LDO); N,N-dilactulose-octamethylenediamine (D-LDO), and N,N-dilactulose-dodecamethylenediamine (D-LDD)), GR-MD-02, GM-CT-01, GCS-100, ipilimumab, a pectin, or a taloside inhibitor may also be used.

In addition, the following sugars may also be used as agents which function similarly to Galectin-8 (as an inhibitor of mTOR) and/or Galectin-9 (an upregulator of AMPKinase). These sugars include, for example, monosaccharides, including f-galactoside sugars, such as galactose, including N- or O-linked (e.g., acetylated) galactosides and disaccharides, oligosaccharides and polysaccharides which contain at least one galactose sugar moiety. These include lactose, mannobiose, melibiose (which may have the glucose residue and/or the galactose residue optionally N-acetylated), melibiulose (which may have the galactose residue optionally N-acetylated), rutinose, (which may have the glucose residue optionally N-acetylated), rutinulose and xylobiose, among others, and trehalose, all of which can be N and O-linked, as well as agarabiose, agarotriose and agarotetraose. Oligosaccharides for use in the present invention as can include any sugar of three or more (up to about 100) individual sugar (saccharide) units as described above (i.e., any one or more saccharide units described above, in any order, especially including galactose units such as galactooligosaccharides and mannan-oligosaccharides ranging from three to about ten-fifteen sugar units in size). Sugars which are galactosides or contain galactose (galactose derivatives) are preferred for use in the present invention. These sugars may function similarly to the galectins, especially galectin-8 (inhibitor of mTOR) or galectin-9 (upregulator of AMPKinase). One or more of these above sugars may be combined with Galectin-8 and/or Galectin-9 or a pharmaceutically acceptable salt or alternative salt thereof and/or a lysosomotropic agent to provide compositions particularly useful in treating an autophagy related disease state or conditions.

Alternatively, one or more sugars described above may function similar to Galectin-8 as an inhibitor of mTOR or Galectin-9 as an upregulator of AMPKinase to be used in combination with a lysosomotropic agent for the treatment of numerous autophagy-related disease states, including cancers. Useful galectin-8-like inhibitors of mTOR or galectin-9 upregulators of AMPKinase include galactoside inhibitors or alternatively, a lactulose amine such as N-lactulose-octamethylenediamine (LDO); N,N-dilactulose-octamethylenediamine (D-LDO), and N,N-dilactulose-dodecamethylenediamine (D-LDD)), GR-MD-02, ipilimumab, a pectin, or a taloside inhibitor, among others.

The term “lysosomotropic agent” is used to describe an agent which is combined with Galectin-8 and/or Galectin-9 or a compound which functions similarly to Galectin-8 as an inhibitor of mTOR or Galectin-9 as an upregulator of AMP kinase to provide compositions according to the present invention which are particularly effective in the treatment of autophagy-related disease states or conditions as otherwise described herein. Lysosomotropic agents include, for example, lipophilic or amphipathic compounds which contain a basic moiety which becomes protonated and trapped in a lysosome. Lysosomotropic agents for use in the present inventon include, for example, lysosomotropic detergents such as a lysosomotropic amine containing a moderately basic amine of pKa 5-9. Examples of such lysosomotropic detergents include sphingosine, O-methyl-serine dodecylamine hydrochloride (MSDH) and N-dodecylimidazole, among others, as well as numerous drugs including chloroquine, chlorpronazine, thioridazine, aripiprazole, clomipramine, imipramine, desipramine and seramasine, among others. Additional lysosomotropic agents include glycyl-L-phenylalanine-2-naphthyl amide (GPN) and Leu-Leu-OMe (LLOMe).

The term “autophagy modulator agent” or “additional autophagy modulator” is used to describe an optional agent which is used in the compositions and/or methods according to the present invention in order to enhance or inhibit an autophagy response in an autophagy mediated disease state which is otherwise treated, ameliorated, inhibited and/or resolved by another agent as set forth herein (e.g. Galectin-8 and/or Galectin-9, a modulator/upregulator of Galectin-8 and/or Galectin-9, or an agent which acts similar to Galectin-8 as an inhibitor of mTOR and/or Galectin-9 as a modulator (upregulator) of AMPKinase or a mixture thereof, optionally in combination with a lysosomotropic agent). Additional autophagy modulators include, but are not limited to, autophagy agonists (such as flubendazole, hexachlorophene, propidium iodide, bepridil, clomiphene citrate (Z,E), GBR 12909, propafenone, metixene, dipivefrin, fluvoxamine, dicyclomine, dimethisoquin, ticlopidine, memantine, bromhexine, ambroxol, norcyclobenzaprine, diperodon, nortriptyline or a mixture thereof or their pharmaceutically acceptable salts). Additional autophagy modulators which may be used in the present invention to inhibit, prevent and/or treat an autophagy mediated disease state and/or condition include one or more of benzethonium, niclosamide, monensin, bromperidol, levobunolol, dehydroisoandosterone 3-acetate, sertraline, tamoxifen, reserpine, hexachlorophene, dipyridamole, harmaline, prazosin, lidoflazine, thiethylperazine, dextromethorphan, desipramine, mebendazole, canrenone, chlotprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, Methimazole, Trimeprazine, Ethoxyquin, Clocortolone, Doxycycline, Pirlindole mesylate, Doxazosin, Deptropine, Nocodazole, Scopolamine, Oxybenzone, Halcinonide, Oxybutynin, Miconazole, Clomipramine, Cyproheptadine, Doxepin, Dyclonine, Salbutamol, Flavoxate, Amoxapine, Fenofibrate, Pimethixene and mixtures thereof. The autophagy modulator may be included as optional agents in compositions according to the present invention or used in conjugation with therapies as otherwise described herein to treat an autophagy mediated disease state or condition.

The term “co-administration” or “combination therapy” is used to describe a therapy in which at least two active compounds in effective amounts are used to treat an autophagy mediated disease state or condition as otherwise described herein, either at the same time or within dosing or administration schedules defined further herein or ascertainable by those of ordinary skill in the art. Although the term co-administration preferably includes the administration of two active compounds to the patient at the same time, it is not necessary that the compounds be administered to the patient at the same time, although effective amounts of the individual compounds will be present in the patient at the same time. In addition, in certain embodiments, co-administration will refer to the fact that two compounds are administered at significantly different times, but the effects of the two compounds are present at the same time. Thus, the term co-administration includes an administration in which one active agent is administered at approximately the same time (contemporaneously), or from about one to several minutes to about 24 hours or more after or before the other active agent is administered.

In yet additional embodiments, additional bioactive agents may be further included in compositions according to the present invention in combination with agents which control mTor response to endomembrane damage (e.g. Galectin-8 and/or Galectin-9, a modulator/upregulator of Galectin-8 and/or Galectin-9, or an agent which acts similar to Galectin-8 as an inhibitor of mTOR and/or Galectin-9 as a modulator (upregulator) of AMPKinase or a mixture thereof, which may optionally be combined with a lysosomotropic agent and/or an autophagy modulator) and may be any bioactive agent such as an additional mTOR inhibitor (i.e., other than Galectin-8) such as Dactolisib (BEZ235, NVP-BEX235, rapamycin, everolimis, AZD8055, Temsilolimus, PI-103, KU0063794, Torkinib (PP242), tacrolimus (FK506), ridaforolimus (deforolimus, MK-8669), Sapanisertib (INK 128), Voxtalisib (XL765), Torin 1, Torin 2, Omipalisib (GSK458), OSI-027, PF-04691502, Apitolisib (RG7422), GSK105%15, Gadatolisib (PKI-587), WYE-354, Vistusertib (AZD2014), WYE-132, BGT226, Palomid 529 (P529), PP121, WYE-687, WAY-600, ETP-46464, GDC-0349, XL-388, CC-115, Zotarolimus (ABT-578), GDC-0084, CZ415, 3DBO, SF2523, MHY1485, Chrysophanic Acid, CC-223, LY3023414, among others, with Torini, Torin 2, pp242, rapamycin/serolimus (which also may function as an autophagy modulator), everolimus, temsirolomis, ridaforolimis, zotarolimis, 32-dexoy-rapamycin, among others being preferred. mTorr inhibitors also include for example, epigallocatechin gallate (EGCG), caffeine, curcumin or reseveratrol (which mTOR inhibitors find particular use as enhancers of autophagy using the compounds disclosed herein). In certain embodiments, an additional mTOR inhibitor as described above, or more often selected from the group consisting of Torin, pp242, rapamycin/serolimus, everolimus, temsirolomis, ridaforolimis, zotarolimis, 32-dexoy-rapamycin, epigallocatechin gallate (EGCG), caffeine, curcumin or reseveratrol and mixtures thereof may be combined with at least one agent selected from the group consisting of digoxin, xylazine, hexetidine and sertindole, the combination of such agents being effective as autophagy modulators in combination.

The terms “cancer” and “neoplasia” are used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated.

As used herein, the terms malignant neoplasia and cancer are used synonymously to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer (especially basal cell carcinoma or squamous cell carcinoma), acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. In certain aspects, the cancer which is treated is lung cancer, breast cancer, ovarian cancer and/or prostate cancer.

Neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms (cancer) are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, stomach and thyroid; leukemias; benign and malignant lymphomas, particularly Burkitts lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991). All of these neoplasms may be treated using compounds according to the present invention.

Representative common cancers to be treated with compounds according to the present invention include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, chorocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. Because of the activity of the present compounds, the present invention has general applicability treating virtually any cancer in any tissue, thus the compounds, compositions and methods of the present invention are generally applicable to the treatment of cancer and in reducing the likelihood of development of cancer and/or the metastasis of an existing cancer.

In certain particular aspects of the present invention, the cancer which is treated is metastatic cancer, a recurrent cancer or a drug resistant cancer, especially including a drug resistant cancer. Separately, metastatic cancer may be found in virtually all tissues of a cancer patient in late stages of the disease, typically metastatic cancer is found in lymph system/nodes (lymphoma), in bones, in lungs, in bladder tissue, in kidney tissue, liver tissue and in virtually any tissue, including brain (brain cancer/tumor). Thus, the present invention is generally applicable and may be used to treat any cancer in any tissue, regardless of etiology.

The term “tumor” is used to describe a malignant or benign growth or tumefacent.

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be an anticancer agent which is distinguishable from a CIAE-inducing anticancer ingredient such as a taxane, vinca alkaloid and/or radiation sensitizing agent otherwise used as chemotherapy/cancer therapy agents herein. In many instances, the co-administration of another anti-cancer compound according to the present invention results in a synergistic anti-cancer effect. Exemplary anti-cancer compounds for co-administration with formulations according to the present invention include anti-metabolites agents which are broadly characterized as antimetabolites, inhibitors of topoisomerase I and II, alkylating agents and microtubule inhibitors (e.g., taxol), as well as tyrosine kinase inhibitors (e.g., surafenib), EGF kinase inhibitors (e.g., tarceva or erlotinib) and tyrosine kinase inhibitors or ABL kinase inhibitors (e.g. imatinib).

Anti-cancer compounds for co-administration include, for example, agent(s) which may be co-administered with compounds according to the present invention in the treatment of cancer. These agents include chemotherapeutic agents and include one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitunumab, amrubicin, oregovomab, Lep-etu, nolatrexed, azd2171, batabulin, otatumumab, zanolimumab, edotecarin, tetrandrine, rubitecan, tesmilifene, oblimersen, ticilimumab, ipilimumab, ossypol. Bio 111, 131-I-TM-601. ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorowidine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemcstane, letrozole, DES(diethylstilbestrol), estradiol, estrogen, conjugated estrogen, bevacizunab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(But)-Leu-Arg-Pro-Azgly-NH₂ acetate [C₅₉H₈₄N₁₈Oi₄-(C₂H₄O₂)_{X} where x=1 to 2A], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TrAK-165, HKI-272, erlotinib, lapatanib, canertinib, ABX-EGF antibody, erbitux, EKB-569, PKI-166, GW-S72016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imtatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezinib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, worunannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilimumab, nivolomuab, pembrolizumab, dabrafenib, trametinib and vemurafenib among others.

Co-administration of one of the formulations of the invention with another anticancer agent will often result in a synergistic enhancement of the anticancer activity of the other anticancer agent, an unexpected result. One or more of the present formulations comprising an IRGM modulator optionally in combination with an autophagy modulator (autostatin) as described herein may also be co-administered with another bioactive agent (e.g., antiviral agent, antihyperproliferative disease agent, agents which treat chronic inflammatory disease, among others as otherwise described herein).

The term “antiviral agent” refers to an agent which may be used in combination with authophagy modulators (autostatins) as otherwise described herein to treat viral infections, especially including HIV infections, HBV infections and/or HCV infections. Exemplary anti-HIV agents include, for example, nucleoside reverse transcriptase inhibitors (NRTI), non-nucleoside reverse transcriptase inhibitors (NNRTI), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development. Exemplary anti-HBV agents include, for example, hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof. Anti-HCV agents include, for example, interferon, pegylated intergeron, ribavirin, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831, F351, NSSA, NS4B, ANA598, A-689, GN1-104, IDX102, ADX184, GL59728, GL60667, PS-7851, TLR9 Agonist, PHX1766, SP-30 and mixtures thereof.

The term “anti-mycobacterial agent” or “anti-tuberculosis agent” shall refer to traditional agents which are used in the art for the treatment of mycobacterial infections, especially including tuberculosis agents. These agents include, for example, one or more of aminosalicyclic acid/aminosalicylate sodium, capreomycin sulfate, clofazimine, cycloserine, ethambutol hydrochloride (myambutol), kanamycin sulfate, pyrazinamide, rifabutin, rifampin, rifapentine, streptomycin sulfate, gatifloxacin and mixtures thereof, all in therapeutically effective amounts, which may be used in conjunction with other agents described herein in the treatment of mycobacterial infections, especially including Mycobacterium tuberculosis (“tuberculosis”) infections.

According to various embodiments, the combination of compositions and/or compounds according to the present invention may be used for treatment or prevention purposes in the form of a pharmaceutical composition. This pharmaceutical composition may comprise one or more of an active ingredient as described herein.

As indicated, the pharmaceutical composition may also comprise a pharmaceutically acceptable excipient, additive or inert carrier. The pharmaceutically acceptable excipient, additive or inert carrier may be in a form chosen from a solid, semi-solid, and liquid. The pharmaceutically acceptable excipient or additive may be chosen from a starch, crystalline cellulose, sodium starch glycolate, polyvinylpyrolidone, polyvinylpolypyrolidone, sodium acetate, magnesium stearate, sodium laurylsulfate, sucrose, gelatin, silicic acid, polyethylene glycol, water, alcohol, propylene glycol, vegetable oil, corn oil, peanut oil, olive oil, surfactants, lubricants, disintegrating agents, preservative agents, flavoring agents, pigments, and other conventional additives. The pharmaceutical composition may be formulated by admixing the active with a pharmaceutically acceptable excipient or additive.

The pharmaceutical composition may be in a form chosen from sterile isotonic aqueous solutions, pills, drops, pastes, cream, spray (including aerosols), capsules, tablets, sugar coating tablets, granules, suppositories, liquid, lotion, suspension, emulsion, ointment, gel, and the like. Administration route may be chosen from subcutaneous, intravenous, intrathecal, intestinal, parenteral, oral, buccal, nasal, intramuscular, transcutaneous, transdermal, intranasal, intraperitoneal, and topical. The pharmaceutical compositions may be immediate release, sustained/controlled release, or a combination of immediate release and sustained/controlled release depending upon the compound(s) to be delivered, the compound(s), if any, to be coadministered, as well as the disease state and/or condition to be treated with the pharmaceutical composition. A pharmaceutical composition may be formulated with differing compartments or layers in order to facilitate effective administration of any variety consistent with good pharmaceutical practice.

The subject or patient may be chosen from, for example, a human, a mammal such as domesticated animal, or other animal. The subject may have one or more of the disease states, conditions or symptoms associated with autophagy as otherwise described herein.

The compounds according to the present invention may be administered in an effective amount to treat or reduce the likelihood of an autophagy-mediated disease and/or condition as well one or more symptoms associated with the disease state or condition. One of ordinary skill in the art would be readily able to determine an effective amount of active ingredient by taking into consideration several variables including, but not limited to, the animal subject, age, sex, weight, site of the disease state or condition in the patient, previous medical history, other medications, etc.

For example, the dose of an active ingredient which is useful in the treatment of an autophagy mediated disease state, condition and/or symptom for a human patient is that which is an effective amount and may range from as little as 100 μg or even less to at least about 500 mg or more, which may be administered in a manner consistent with the delivery of the drug and the disease state or condition to be treated. In the case of oral administration, active is generally administered from one to four times or more daily. Transdermal patches or other topical administration may administer drugs continuously, one or more times a day or less frequently than daily, depending upon the absorptivity of the active and delivery to the patient's skin. Of course, in certain instances where parenteral administration represents a favorable treatment option, intramuscular administration or slow IV drip may be used to administer active. The amount of active ingredient which is administered to a human patient is an effective amount and preferably ranges from about 0.05 mg/kg to about 20 mg/kg, about 0.1 mg/kg to about 7.5 mg/kg, about 0.25 mg/kg to about 6 mg/kg., about 1.25 to about 5.7 mg/kg.

The dose of a compound according to the present invention may be administered at the first signs of the onset of an autophagy mediated disease state, condition or symptom. For example, the dose may be administered for the purpose of lung or heart function and/or treating or reducing the likelihood of any one or more of the disease states or conditions which become manifest during an inflammation-associated metabolic disorder or tuberculosis or associated disease states or conditions, including pain, high blood pressure, renal failure, or lung failure. The dose of active ingredient may be administered at the first sign of relevant symptoms prior to diagnosis, but in anticipation of the disease or disorder or in anticipation of decreased bodily function or any one or more of the other symptoms or secondary disease states or conditions associated with an autophagy mediated disorder to condition.

The present invention thus relates to the following embodiments, among others.

A method of treating an autophagy mediated disease in a patient in need comprising administering to the patient an effective amount of Galectin-8 and/or Galectin-9, a modulator/upregulator of Galectin-8 and/or Galectin-9, or an agent which acts similar to Galectin-8 as an inhibitor of mTOR and/or Galectin-9 as a modulator (upregulator) of AMPKinase or a mixture thereof, optionally in combination with a lysosomotropic agent.

The method wherein the upregulator of galectin-8 or Galectin-9 or the agent which acts similarly to Galectin-8 and/or Galectin-9 is a sugar which comprises at least one galactose unit.

The method wherein the sugar is selected from a monosaccharide, including β-galactoside sugars, such as galactose, including N- or O-linked galactosides and disaccharides, oligosaccharides and polysaccharides which contain at least one galactose unit.

The method wherein the sugar is galactose, a galactoside, lactose, mannobiose, melibiose, melibiulose (which may have the galactose residue optionally N-acetylated), rutinose, rutinulose, xylobiose, and trehalose, all of which optionally comprise N and O-linked acetyl groups.

The method wherein the sugar is an oligosaccharide containing at least one galactose unit.

The method wherein the sugar is a galactooligosaccharide ranging from three to about fifteen galactose units in size.

The method wherein the sugar is a galactoside or is a galactose derivative.

The method wherein the agent which acts similar to Galectin-8 or Galectin-9 or upregulates Galectin-8 or Galectin-9 is a lactulose amine such as N-lactulose-octamethylenediamine (LDO); N,N-dilactulose-octamethylenediamine (D-LDO), and N,N-dilactulose-dodecamethylenediamine (D-LDD)), GR-MD-02, ipilimumab, a pectin, or a taloside inhibitor.

The method wherein the composition includes a lysosomotropic agent.

The method wherein the lysosomotropic agent is a lipophilic or amphipathic compound which contains a basic moiety which becomes protonated and trapped in a lysosome.

The method the lysosomotropic agent is a lysosomotropic detergent.

The method wherein the lysosomotropic detergent is a lysosomotropic amine containing a moderately basic amine of pKa 5-9.

The method wherein the lysosomotropic amine is sphingosine, O-methyl-serine dodecylamine hydrochloride (MSDH), N-dodecylimidazole, or a mixture thereof.

The method wherein the lysomotropic agent is chloroquine, chlorpromazine, thioridazine, aripiprazole, clomipramine, imipramine, desipramine, seramasine, or a mixture thereof.

The method wherein the lysosomotropic agent is glycyl-L-phenylalanine-2-naphthyl amide (GPN), Leu-Leu-OMe (LLOMe) or a mixture thereof.

The method wherein the autophagy mediated disease state is a metabolic syndrome disease, a microbial infection, an inflammatory disorder, a lysosomal storage disorder, an immune disorder, cancer or a neurodegenerative disorder.

The method wherein the microbial infection is a Mycobacterium infection.

The method wherein the Mycobacterium infection is a M. tuberculosis infection.

The method wherein the autophagy mediated disease state is cancer.

The method further including an additional cancer agent to treat the cancer.

The method further including administering at least one additional agent selected from the group consisting of an additional autophagy modulator and/or at least one compound selected from the group consisting of Torin, pp242, rapamycin/serolimus (which also may function as an autophagy modulator), everolimus, temsirolomis, ridaforolimis, zotarolimis, 32-dexoy-rapamycin, epigallocatechin gallate (EGCG), caffeine, curcumin, reseveratrol or mixtures thereof.

The method wherein the autophagy mediated disease state is a metabolic syndrome disease, an infectious disease, a lysosome storage disease, cancer or an aging related disease or disorder.

The method wherein the autophagy mediated disease state is Alzheimer's disease, Parkinson's disease, Huntington's disease; inflammatory bowel disease, including Crohn's disease, rheumatoid arthritis, lupus, multiple sclerosis, chronic obstructive pulmonary disease/COPD, pulmonary fibrosis, cystic fibrosis, Sjogren's disease; hyperglycemic disorders, diabetes (I and II), severe insulin resistance, hyperinsulinemia, insulin-resistant diabetes, dyslipidemia, depressed high-density lipoprotein (HDL), and elevated triglycerides, liver disease, renal disease, cardiovascular disease, including infarction, ischemia, stroke, pressure overload and complications during reperfusion, muscle degeneration and atrophy, symptoms of aging, low grade inflammation, gout, silicosis, atherosclerosis, age-associated dementia and sporadic form of Alzheimer's disease, psychiatric conditions including anxiety and depression, spinal cord injury, arteriosclerosis or a bacterial, fungal, cellular or viral infections.

The method wherein the autophagy mediated disease state is activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and II), GM Ganliosidosis, including infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs or Wolman disease.

The method wherein the autophagy mediated disease state is myocarditis, Anti-glomercular Base Membrane Nephritis, lupus erythematosus, lupus nephritis, autoimmune hepatitis, primary biliary cirrhosis, alopecia areata, autoimmune urticaria, bullous pemphagoid, dermatitis herpetiformis, epidermolysis bullosa acquisita, linear IgA disease (LAD), pemphigus vulgaris, psoriasis, Addison's disease, autoimmune polyendocrine syndrome I, II and III (APS I, APS II, APS III), autoimmune pancreatitis, type I diabetes, autoimmune thyroiditis, Ord's thyroiditis, Grave's disease, autoimmune oophoritis, Sjogren's syndrome, autoimmune enteropathy, Coeliac disease, Crohn's disease, autoimmune hemolytic anemia, autoimmune lymphoproliferative syndrome, autoimmune neutropenia, autoimmune thrombocytopenic purpura, Cold agglutinin disease, Evans syndrome, pernicious anemia, Adult-onset Still's disease, Felty syndrome, juvenile arthritis, psoriatic arthritis, relapsing polychondritis, rheumatic fever, rheumatoid arthritis, myasthenia gravis, acute disseminated encephalomyelitis (ADEM), balo concentric sclerosis, Guillain-Barre syndrome, Hashimoto's encephalopathy, chronic inflammatory demvelinating polyneuropathy, Lambert-Eaton myasthenic syndrome, multiple sclerosis, autoimmune uveitis. Graves opthalmopathy, Granulomatosis with polyangitis (GPA), Kawasaki's disease, vasculitis or chronic fatigue syndrome.

The method wherein the autophagy-related disease state or condition is a metabolic syndrome disease.

The method wherein the autophagy-related disease state or condition is an aging related disease or disorder.

Other embodiments of the present invention relate to pharmaceutical compositions including:

A pharmaceutical composition comprising an effective amount of Galectin-8 and/or Galectin-9, a modulator/upregulator of Galectin-8 and/or Galectin-9, or an agent which acts similar to Galectin-8 as an inhibitor of mTOR and/or Galectin-9 as a modulator (upregulator) of AMPKinase or a mixture thereof, optionally in combination with a lysosomotropic agent.

The composition wherein the upregulator of galectin-8 or Galectin-9 or said agent which acts similarly to Galectin-8 and/or Galectin-9 is a sugar which comprises at least one galactose unit.

The composition wherein the sugar is selected from a monosaccharide, including β-galactoside sugars, such as galactose, including N- or O-linked galactosides and disaccharides, oligosaccharides and polysaccharides which contain at least one galactose unit.

The composition wherein the sugar is galactose, a galactoside, lactose, mannobiose, melibiose, melibiulose (which may have the galactose residue optionally N-acetylated), rutinose, rutinulose, xylobiose or trehalose, all of which optionally comprise N and O-linked acetyl groups.

The composition wherein the sugar is an oligosaccharide containing at least one galactose unit.

The composition wherein the sugar is a galactooligosaccharide ranging from three to about ten-fifteen galactose units in size.

The composition wherein the sugar is a galactoside or is a galactose derivative.

The composition wherein the agent which acts similar to Galectin-8 or Galectin-9 or upregulates Galectin-8 or Galectin-9 is a lactulose amine such as N-lactulose-octamethylenediamine (LDO); N,N-dilactulose-octamethylenediamine (D-LDO), and N,N-dilactulose-dodecamethylenediamine (D-LDD)), GR-MD-02, ipilimumab, a pectin, or a taloside inhibitor.

The composition which includes a lysosomotropic agent.

The composition wherein the lysosomotropic agent is a lipophilic or amphipathic compound which contains a basic moiety which becomes protonated and trapped in a lysosome.

The composition wherein the lysosomotropic agent is a lysosomotropic detergent.

The composition wherein the lysosomotropic detergent is a lysosomotropic amine containing a moderately basic amine of pKa 5-9.

The composition wherein the lysosomotropic amine is sphingosine, O-methyl-serine dodecylamine hydrochloride (MSDH), N-dodecylimidazole or a mixture thereof.

The composition wherein the lysomotropic agent is chloroquine, chlorpromazine, thioridazine, aripiprazole, clomipramine, imipramine, desipramine, seramasine, or a mixture thereof.

The composition wherein the lysosomotropic agent is glycyl-L-phenylalanine-2-naphthyl amide (GPN), Leu-Leu-OMe (LLOMe) or a mixture thereof.

The composition which further includes an additional autophagy modulator and/or at least one compound selected from the group consisting of Torin, pp242, rapamycin/serolimus (which also may function as an autophagy modulator), everolimus, temsirolomis, ridaforolimis, zotarolimis, 32-dexoy-rapamycin, epigallocatechin gallate (EGCG), caffeine, curcumin, reseveratrol or mixtures thereof.

These and other aspects and embodiments of the invention described above, are described further in the following illustrative examples which are provided for illustration of the present invention and are not to be taken to limit the present invention in any way.

### EXAMPLES

### Method Details

### Antibodies and Reagents

Antibodies were from Cell Signaling Technology (CST) were phospho-T389 S6K1 (108D2, #9234) (1:1000 for Western blot (WB)), S6K1 (49D7, #2708) (1:1000 for WB), phospho-S757 ULK1 (#6888)(1:1000 for WB), phospho-S317 ULK1 (D2B6Y, #12753)(1:1000 for WB), ULK1 (D8H5, #8054) (1:1000 for WB), TSC2 (D93F12, #4308)(1:1000 for WB), RagA (D8B5, #4357) (1:1000 for WB), RagB (D18F3, #8150)(1:1000 for WB), RagC (#3360)(1:1000 for WB), RagD (#4470)(1:1000 for WB), LAMTOR1 (D11H6, #8975) (1:1000 for WB), LAMTOR2 (D7C10, #8145)(1:1000 for WB), LAMTOR3 (D38G5, #8168) (1:1000 for WB), mTOR (7C10, #2983)(1:1000 for WB; 1:400 for immunofluorescence (IF)), Raptor (24C12, #2280)(1:1000 for WB), TFEB (#4240)(1:1000 for WB; 1:200 for IF), ATG13 (E1Y9V, #13468) (1:100 for IF), TAK1 (#4505)(1:1000 for WB), AMPKα (#2532) (1:1000 for WB), phospho-T172 AMPKα (40119, #2535)(1:1000 for WB), Acetyl-CoA Carboxylase (#3662)(1:1000 for WB), phospho-S79 Acetyl-CoA Carboxylase (#3661)(1:1000 for WB). Other antibodies used in this study were from the following sources: FLAG M2 (F1804)(1:1000 for WB), LC3B (L7543)(1:1000 for WB), SLC38A9 (HPA043785)(1:1000 for WB) from Sigma Aldrich; GFP (ab290)(1:1000 for WB), beta-Tubulin (ab6046)(1:1000 for WB), Galectin-9 (ab69630)(1:1000 for WB) from Abcam, LKB1 (ab61122)(1:1000 for WB), CAMKK2 (ab168818)(1:1000 for WB); Galectin-8 (H-80) (1:200 for WB), Galectin-3 (SC-32790)(1:200 for WB) and beta-Actin (C4) (1:1000 for WB) from Santa Cruz Biotechnology; LC3 (PM036)(1:500 for IF) from MBL International; LAMP2 (H4B4)(1:500 for IF) from DSHB of University of Iowa; HRP-labeled anti-rabbit (1:2000 for WB) and anti-mouse (1:2000 for WB) secondary antibodies from Santa Cruz Biotechnology; Clean-Blot IP Detection Kit (HRP) (21232)(1:1000 for WB), Alexa Fluor 488, 568 (1:500 for IF) from ThermoFisher Scientific.

Reagents used in this study were from the following sources: Streptavidin Magnetic Beads (88816), Dynabeads Protein G (10003D) from ThermoFisher Scientific; Gly-Phe-beta-Napthylamide (GPN)(21438-66-4) from Cayman Chemicals; Blotinyl tyramide (biotin-phenol) (CDX-B0270-M100) from AdipoGen; sodium ascorbate (A7631), sodium azide (S2002), Trolox (238813) and Leu-Leu-methyl ester hydrobromide (LLOMe, L7393) from Sigma Aldrich; Urea (17-1319-01) from Pharmabiotech; DMEM, RPMI and EBSS medias from Life Technologies; PNGasF from New England Biolabs.

### Cells and Cell Lines

HEK293T, HeLa and THP-1 cells were from ATCC. Bone marrow derived macrophages (BMMs) were isolated from femurs of Atg5^{fl/fl} LysM-Cre mice or Gal8 Atg5^{fl/fl} LysM-Cre and their Cre-negative litermates, and cultured in DMEM supplemented with mouse macrophage colony stimulating factor (mM-CSF, #5228, CST). THP-1 cells were differentiated with 50 nM phorbol 12-myristate 13-acetate (PMA) overnight before use. Glucose starvation was performed by glucose-free medium (ThermoFisher, #11966025) supplemented with 10/fetal bovine serum (FBS). TSC2-knockout HeLa cells and SLC38A9-knockout HEK293T cells were from David M. Sabatini (Whitehead Institute). HEK293T cells stably expressing FLAG-metap2/FLAG-p14 and constitutively active RagB^{Q99L} were from Roberto Zoncu (UC Berkeley).

### Cultured Human Peripheral Blood Monocyte Cells

A 40-50 mL blood draw was collected from a healthy, consenting adult volunteer enrolled in our HRRC-approved study by a trained phlebotomist. Keeping different donors separate, blood in 10 mL vacutainers was pooled into 2-50 mL conicals, the volume brought to 50 mL with sterile 1×PBS and mixed by inversion. 25 mL of the blood mix were carefully layered onto 20 mL of Ficoll (Sigma, #1077) in separate conical tubes and centrifuged at 2000 rpm for 30 min at 22° C. The buffy layer containing human peripheral blood monocytes (PBMCs) was removed, pooled, washed with 1×PBS twice and resuspended in ˜20 mL RPM media with 10% human AB serum and Primocin.

Plasmids, siRNAs, and Transfection

The following plasmids were from Addgene: pRK5-HA GST RagA (#19298), pRK5-HA GST RagD (#19307), pRK5-HA GST RagA 21L (#19299), pRK5-HA GST RagA 66L (#19300), pRK5-HA GST RagD 77L (#19308), pRK5-HA GST RagD 121L (#19309), pRK5-HA GST RagB (#19301), pRK5HA GST RagC (#19304), pRK5-HA GST RagB 99L (#19303), pRK5-HA GST RagB 54L (#19302), pRK5-HA GST RagC 75L (#19305), pRK5-HA GST RagC 120L (#19306), pRK5-p18-FLAG (#42331), pRK5-FLAG-SLC38A9.1 (#71855), pcDNA3 APEX2-NES (#49386), pDONR221-metap2 (HsCD00043030) was from DNASU. Plasmids used in this study, such as LAMTOR1/p18, RagA, B, C or D and the corresponding mutants, were cloned into pDONR221 using BP cloning, and expression vectors were made utilizing LR cloning (Gateway, ThermoFisher Scientific) in appropriate pDEST vectors for immunoprecipitation or GST-pulldown assay.

The Gateway Vector Conversion System (ThermoFisher Scientific) was used to construct pJJiaDEST-APEX2. Galectin-8 mutants were generated utilizing the QuikChange site-directed mutagenesis kit (Agilent) and confirmed by sequencing (Genewiz). YFP-fused galectins were from Felix Randow (MRC Laboratory of Molecular Biology, UK). All siRNAs were from GE Dharmacon. Plasmid transfections were performed using the ProFection Mammalian Transfection System (Promega) or Amaxa nucleofection (Lonza). siRNAs were delivered into cells using either Lipofectamine RNAiMAX (ThermoFisher Scientific) or Amaxa nucleofection (Lonza).

### High Content Microscopy

Cells in 96 well plates were treated, followed by fixation in 4% paraformaldehyde for 5 min. Cells were then permeabilized with 0.1% saponin in 3% Bovine serum albumin (BSA) for 30 min followed by incubation with primary antibodies for 2 h and secondary antibodies for 1 h. High content microscopy with automated image acquisition and quantification was carried out using a Cellomics HCS scanner and iDEV software (ThermoFisher Scientific). Automated epifluorescence image collection was performed using a minimum of 500 cells per well. Epifluorescence images were machine analyzed using preset scanning parameters and object mask definitions. Hoechst 33342 staining was used for autofocus and to automatically define cellular outlines based on background staining of the cytoplasm. Primary objects were cells, regions of interest (ROI) or targets were algorithm-defined for shape/segmentation, maximum/minimum average intensity, total area and total intensity minima and maxima limits, etc., to automatically identify puncta or other profiles within valid primary objects. Nuclei were defined as a region of interest for TFEB translocation. All data collection, processing (object, ROI, and target mask assignments) and analyses were computer driven independently of human operators.

### Immunofluorescence Confocal Microscopy and Analysis

HeLa or HEK293T cells were plated onto coverslips in 6-well plates. After treatment, cells were fixed in 4% paraformaldehyde for 5 min followed by permeabilization with 0.1% saponin in 3% BSA for 30 min. Cells were then incubated with primary antibodies for 2 h and appropriate secondary antibodies Alexa Fluor 488 or 568 (ThermoFisher Scientific) for 1 h at room temperature. Coverslips were mounted using Prolong Gold Antifade Mountant (ThermoFisher Scientific). Images were acquired using a confocal microscope (META; Carl Zeiss) equipped with a 63×/1.4 NA oil objective, camera (LSM META; Carl Zeiss), and AIM software (Carl Zeiss).

### Co-Immunoprecipitation Assay

Cells transfected with 8-10 μg of plasmids were lysed in NP-40 buffer (ThermoFisher Scientific) supplemented with protease inhibitor cocktail (Roche, 11697498001) and 1 mM PMSF (Sigma, 93482) for 30 min on ice. Supernatants were incubated with (2-3 μg) antibodies overnight at 4° C. The immune complexes were captured with Dynabeads (ThermoFisher Scientific), followed by three times washing with 1×PBS. Proteins bound to Dynabeads were eluted with 2× Laemmli sample buffer (Biorad) and subjected to immunoblot analysis.

### APEX2-Labeling and Streptavidin Enrichment for Immunoblotting Analyses

HEK293T cells transfected with pJJiaDEST-APEX2 or pJJiaDEST-APEX2-Gal8 were incubated with 100 μM GPN (Cayman Chemicals) in full medium for 1 h (confluence of cells remained at 70-80%). Cells were next incubated in 500 μM biotin-phenol (AdipoGen) in full medium for the last 30 min of GPN incubation. A 1 min pulse with 1 mM H₂O₂ at room temperature was stopped with quenching buffer (10 mM sodium ascorbate, 10 mM sodium azide and 5 mM Trolox in Dulbecco's Phosphate Buffered Saline (DPBS)). All samples were washed twice with quenching buffer, and twice with DPBS.

For immunoblotting analysis, cell pellets were lysed with 500 μL ice-cold RIPA lysis buffer (ThermoFisher Scientific) with protease inhibitor cocktail (Roche), 1 mM PMSF (sigma), 10 mM sodium ascorbate, 10 mM sodium azide and 5 mM Trolox, gently pipetted and then the incubated for 30 min. The lysates were clarified by centrifugation at 13,000 rpm for 5 min, followed by measuring protein concentrations using a Pierce BCA Protein Assay kit, with freshly made bovine serum albumin (BSA) solutions as standards. Streptavidin-coated magnetic beads (ThermoFisher Scientific) were washed with RIPA lysis buffer. 3 mg of each sample was mixed with 100 μL of streptavidin beads. The suspensions were gently rotated at 4° C. overnight to bind biotinylated proteins. The flowthrough was removed, and the beads were washed twice with 1 mL RIPA lysis buffer, 1 mL of 2 M urea (Pharmabiotech) in 10 mM Tris-HCl (PH8.0), and again twice with 1 mL RIPA lysis buffer. Beads-bound biotinylated proteins were desorbed from beads by heating the beads at 100° C. for 10 min in 30 μL 2×Laemmli sample buffer (Biorad) supplemented with 2 mM biotin (Sigma). 15 μL of each sample was separated by SDS-PAGE and analyzed by Western blot using indicated antibodies.

For LC-MS/MS analysis, cell pellets were lysed in 500 μL ice-cold lysis buffer (6 M urea, 0.3 M Nacl, 1 mM EDTA, 1 mM EGTA, 10 mM sodium ascorbate, 10 mM sodium azide, 5 mM Trolox, 1% glycerol and 25 mm Tris/HCl [PH 7.5]) for 30 min by gentle pipetting. Lysates were clarified by centrifugation and protein concentrations determined as above. Streptavidin-coated magnetic beads (Pierce) were washed with lysis buffer. 3 mg of each sample was mixed with 100 μL of streptavidin bead. The suspensions were gently rotated at 4° C. for overnight to bind biotinylated proteins. The flowthrough after enrichment was removed and the beads were washed in sequence with 1 mL IP buffer (150 mM NaCl, 10 mM Tris-HCl pH8.0, 1 mM EDTA, 1 mM EGTA, 1% Triton X-100) twice; 1 mL 1M KCl; 1 mL of 50 mM Na₂CO₃; 1 mL 2M Urea in 20 mM 1Tris HCl pH8; 1 mL IP buffer. Biotinylated proteins were eluted, 5-10% of the sample processed for Western Blot and 90-95% of the sample processed for mass spectrometry.

### LC-MS/MS

Digested peptides were analyzed by LC-MS/MS on a Thermo Scientific Q Exactive Plus Orbitrap Mass spectrometer in conjunction Proxeon Easy-nLC II HPLC (Thermo Scientific) and Proxeon nanospray source. The digested peptides were loaded a 100 micron×25 mm Magic C18 100 Å 5U reverse phase trap where they were desalted online before being separated using a 75 micron×150 mm Magic C18 200 Å 3U reverse phase column. Peptides were eluted using a 140 minute gradient with a flow rate of 300 nl/min. An MS survey scan was obtained for the m/z range 350-1600, MS/MS spectra were acquired using a top 15 method, where the top 15 ions in the MS spectra were subjected to HCD (High Energy Collisional Dissociation). An isolation mass window of 1.6 m/z was for the precursor ion selection, and normalized collision energy of 27% was used for fragmentation. A fifteen-second duration was used for the dynamic exclusion.

### GST-Pulldown Assay

GST and GST-tagged proteins were produced in SoluBL21 Competent E. coli (Genlantis, C700200) and purified by binding to Glutathionine Sepharose 4 Fast Flow beads (GE Healthcare, 17-5132-01) while myc-tagged proteins were in vitro translated using the TNT T7 Reticulocyte Lysate System (Promega, 14610) in the presence of ³⁵S-methionine. 10 μL of translated protein were incubated with immobilized GST-tagged protein in NETN-buffer (50 mM Tris pH 8.0, 150 mM NaCl, 1 mM EDTA, 0.5% NP-40) supplemented with complete Mini EDTA-free protease inhibitor cocktail tablets (Roche, 11836170001, 1 tablet/10 mL) for 1 h at 4° C. followed by five times washing with NETN buffer. 2×SDS gel loading buffer were added and protein separated by SDS-PAGE. Gels were stained with Coomassie Brilliant Blue R-250 Dye (ThermoFisher Scientific, 20278) to visualize the fusion proteins. Radioactive signals were detected by Fujifilm bioimaging analyzer BAS-5000 and quantified with ScienceLab ImageGuage software (Fujifilm).

### Generation of Gal3. Gal CRISPR and Gal9 CRISPR Mutant Cells

Gal3/8-depleted cells were generated with CRISPR/Cas9-mediated knockout system, HeLa cells were transfected with a Gal3/8 CRISPR/Cas9 KO plasmid purchased from Santa Cruz Biotechnology, sc-417680/401785). Human Gal3 target sequence was a pool of 3 different gRNA plasmids (gRNA: CAGCTCCATGATGCGTTATC; gRNA2: CAGACCCAGATAACGCATCA; gRNA3: CGGTGAAGCCCAATGCAAAC) and human Gal8 target sequence was a pool of 3 different gRNA plasmids (gRNA1: CATGAAACCTCGAGCCGATG; gRNA2: ATGTTCCTAGTGACGCAGAC; gRNA3: CGTATCACAATCAAAGTTCC) located within the coding DNA sequence fused to Streptococcus pyogenes Cas9, and GFP. Transfected cells (green fluorescence) were sorted by flow cytometry and single-cell clones analyzed by immunoblotting for a loss of Gal3/8 band (FIG. S4H/A).

For Gal9, the lentiviral vector lentiCRISPRv2 carrying both Cas9 enzyme and a gRNA targeting Gal9 (gRNA target sequence: ACACACACACCTGGTTCCAC) was transfected into HEK293T cells together with the packaging plasmids psPAX2 and pCMV-VSV-G at the ratio of 5:3:2. Two days after transfection, the supernatant containing lentiviruses was collected and used to infect HEK293A cells. 36 hours after infection, the cells were selected with puromycin (1 mg/mL) for one week in order to select Gal9-knockout cells. Gal9 knockout was confirmed by Western blot. Selection of single clones was performed by dilution in 96-well, which were confirmed by Western blots (FIG. S5D).

### Quantification and Statistical Analysis

### Mass Spectrometer Data Processing and Analysis

Tandem mass spectra were extracted by Proteome Discoverer version 2.2. Charge state deconvolution and deisotoping were not performed. All MS/MS samples were analyzed using Sequest-HT (XCorr Only)(Thermo Fisher Scientific, San Jose, Calif., USA; in Proteome Discoverer 2.2.0.388). Sequest (XCorr Only) was set up to search the gpm common laboratory contaminants and the Uniprot human proteome 3AUP000005640 with isoforms (August 2017, 93299 entries) assuming the digestion enzyme trypsin. Sequest (XCorr Only) was searched with a fragment ion mass tolerance of 0.020 Da and a parent ion tolerance of 10.0 PPM. Carbamidomethyl of cysteine was specified in Sequest (XCorr Only) as a fixed modification. Deamidated of asparagine, oxidation of methionine and acetyl of the n-terminus were specified in Sequest (XCorr Only) as variable modifications. Precursor intensity was determined using Proteome Discoverer 2.2 using the Minor Feature detector with the default options.

Scaffold (version Scaffold 4.8.2, Proteome Software Inc., Portland, Oreg.) was used to validate MS/MS based peptide and protein identifications. Peptide identifications were accepted if they could be established at greater than 93.0% probability to achieve an FDR less than 0.1% by the Scaffold Local FDR algorithm. Protein identifications were accepted if they could be established at greater than 99.0% probability and contained at least two identified peptides. This filtering resulted in a decoy false discovery rate of 0.08% on the spectra level and 0.7% on the protein level. Protein probabilities were assigned by the Protein Prophet algorithm. Proteins that contained similar peptides and could not be differentiated based on MS/MS analysis alone were grouped to satisfy the principles of parsimony. Proteins sharing significant peptide evidence were grouped into clusters. Complete MS/MS proteomic data have been deposited at MassIVE, ID MSV000081788 and linked to ProteomeXchange accession ID PXD008390.

Data in this study are presented as means±SEM (n≥3). Data were analyzed with either analysis of variance (ANOVA) with Tukey's HSD post-hoc test, or a two-tailed Student's t test. Animal survival data were analyzed by log-rank (Mantel-Cox) method. Statistical significance was defined as: † (not significant) p≥0.05 and *p<0.05, **p<0.01.

### Data and Software Availability

The mass spectrometry ProteomeXchange (website: proteomexchange.org)) dataset PXD008390 reported in this study has been deposited in MassIVE repository (https://massive.ucsd.edu) with the accession number for MSV000081788. Original source files (microscopy images and western blots) have been deposited in Mendeley as a Dataset available at the website (http://data.mendey.com/datasets/m6jwzzt3g/draft?a=f1533de-0864-4300-bed4-a6169d4a0158), which is incorporated herein. See also, Jia, et al., Molecular Cell, 70(1), pp. 120-135, Apr. 15, 2018 and Jia, et al., Autophagy, Aug. 6, 2018 (posted online), the entire contents of both of said publications being incorporated by reference herein.

### APEX2-Ga/8 Proximity-Biotinylation LC/MS/MS Proteomic Analysis

HEK293T cells transfected with pJJiaDEXT-APEX2-Gal8 (APEX2 fusion with LGALS8) were incubated in full medium with (plus +GPN) or without (minus, −GPN) 100 μM GPN for 1 h, processed for and subjected to LC/MS/MS as described in START method, proteomic data analyses. Three repeats in treatment-control pairs are marked as follows (i-iii): (i) gpn1 (minus, full medium control), and gpn2 (plus, 100 μM GPN); (ii) gpn3 (minus, full medium control) and gpn4 (plus, 100 μM GPN); (iii) gpn5 (minus, full medium control) and gpn6 (plus, 100 μM GPN). The MS/MS proteomic data (ProteomeXchange Accession ID PXD008390) have been deposited at MassIVE, ID MSV000081788. See also FIG. 6G hereof.

### Results

Lysosomal Damage Inhibits mTOR Signaling

The mTORC1 complex localizes to lysosomes (Kim, et al., 2008; Sancak, et al., 2008) where it responds to nutrient inputs (Castellano, et al., 2017; Saxton and Sabatini, 2017). The inventors wondered, whether mTORC1 was also affected by the lysosomal membrane integrity. Herein, mTORC1 is referred to primarily as mTOR as the inventors have not monitored all components of mTORC1 in all experiments. Using GPN to induce lysosomal damage (Berg, et al., 1994), we observed diminished mTOR activity as detected by phosphorylation of its substrates S6K1 and ULK1, in a dose response manner (FIG. 1A). This effect was reversed upon GPN washout (FIG. 1B). GPN inhibited mTOR activity in different cell lines tested and was comparable to the effects of starvation (FIG. S1A). Similarly, another lysosomal damaging agent LLOMe (Aits, et al., 2015; Thiele and Lipsky, 1990), caused inhibition of mTOR activity (FIG. S1B). A non-enzymatic, physical membrane/lysosomal damaging agent, silica (Hornung, et al., 2008), also reduced mTOR activity (FIG. 1C). The above agents caused lysosomal damage as reflected in diminished LysoTracker Red DND-99 staining (FIG. S1C). The effects of GPN on mTOR were confirmed in primary cells, using human peripheral blood monocyte derived macrophages (FIG. 1D). mTOR, translocated from lysosomes to the cytosol upon treatment with GPN (FIGS. 1E, F and S1E), LLOMe, or silica (FIGS. 1E and S1D, E). LLOMe washout allowed relocalization of mTOR to lysosomes (FIG. S1F). Lysosomal damage resulted in functional responses downstream of mTOR. TFEB, a transcriptional regulator controlling expression of the lysosomal/autophagosomal systems (Napolitano and Ballabio, 2016), translocated to the nucleus from the cytoplasm in cells treated with GPN, LLOMe, or silica, comparably to the effects of starvation (FIGS. 1G and S1G). Autophagy, normally repressed by mTOR (Kim, et al., 2011), was activated as well, as indicated by increase in LC3 puncta (FIG. 1H) and LC3 lipidation (FIG. S1H) and by an increase in ATG13 puncta, a marker specific for autophagy initiation independent of the flux (Karanasios, et al., 2016)(FIGS. 11 and S1I). Thus, lysosomal damage inactivates mTOR (FIG. S1J).

Ragulator-Rag Complex Responds to Lysosomal Damage in Control of mTOR

The inventors investigated systems (Saxton and Sabatini, 2017) responsible for transducing lysosomal damage signals to mTOR. The tuberous sclerosis complex (TSC) includes TSC2, a GAP inactivating the GTPase Rheb (Inoki, et al., 2003; Tee, et al., 2003), which in turn activates mTOR (Long, et al., 2005; Sancak, et al., 2007). We tested TSC-Rheb pathway, and found that GPN inhibited mTOR even in cells null for TSC2 (Castellano, et al., 2017) (FIG. 2A), indicating that lysosomal damage inactivates mTOR independently of TSC2. Another system, the Ragulator-Rag complex on lysosomes, transduces amino acid abundance (Sancak, et al., 2010) and cholesterol (Castellano, et al., 2017) signals to mTOR. This system consists of the pentameric Ragulator complex (including LAMTOR1/p18 and LAMTOR2/p4) functioning as a GEF acting upon a quartet of small GTPases, RagA, B, C and D. The Ragulator-Rag interaction increases during amino acid starvation (Bar-Peled, et al., 2012) or cholesterol depletion (Castellano, et al., 2017), believed to reflect increased affinity of GEFs (in this case Ragulator) for inactive (GDP-bound) cognate GTPases such as Rags (Bar-Peled et al., 2012; Castellano, et al., 2017; Zoncu, et al., 2011). Thus, we tested whether this system is responsive to lysosomal damage signals. First, as a control, lysosomal damage did not cause disturbance in lysosomal localization of Ragulator (p18) or RagC (FIG. S2A). Second, when we quantified in co-IPs the interactions between Ragulator and Rags, as an established readout for the activation state of Rags (Bar-Peled et al., 2012; Castellano, et al., 2017; Zoncu, et al., 2011), treatment with GPN enhanced interactions between Ragulator (p14 and p18) and RagA/RagC (FIGS. 2B and S2B).

Activated Rags bind Raptor and recruit mTOR to the lysosomes (Saxton and Sabatini, 2017). GPN treatment diminished mTOR and Raptor levels in complexes with RagB (FIG. 2Q or RagA (FIG. S2C) indicative of RagA/B being in an inactive state upon lysosomal damage. Whereas GPN treatment diminished mTOR activity monitored by phosphorylation levels of S6K1, this was not the case in cells stably expressing constitutively active form of RagB (RagB^{Q99L}) (FIG. 2D). Moreover, mTOR remained on lysosomes in cells expressing RagB^{Q99L} treated with GPN (FIGS. 2E, F and S2D). These relationships are summarized in FIG. S2E.

Galectin is in Dynamic Complexes with mTOR and its Regulators

How might lysosomal damage be transduced to the Ragulator-Rag system to control mTOR? Galectins, a family of cytosolic lectins (Arthur, et al., 2015), can detect endomembrane injury such as the damage artificially caused by LLOMe (Aits, et al., 2015) or physiologically during sterile or infection-associated damage of endosomal, phagosomal, and lysosomal membranes (Aits, et al, 2015; Chauhan, et al., 2016; Fujita, et al., 2013; Thurston, et al., 2012). Since the Ragulator-Rag system and mTOR are localized on lysosomes, we wondered whether there is a connection between galectins and mTOR regulation. We screened a set of galectins for response to GPN, and observed galectin puncta formation, previously reported to be on lysosomes (Aits et al., 2015), with Gal3, Gal8, Gal9 and, to a lesser extent, galectin-12 (FIG. 3A). This pattern was similar, with the exception of galectin-12, to the one observed with Salmonella-induced vesicle damage (Thurston, et al., 2012). The two strongest responders, Gal8 and Gal9, formed puncta in response to GPN-induced lysosomal damage but not upon treatment with chloroquine, an acidotropic compound known to neutralize lysosomal pH, or Bafilomycin A 1, an inhibitor of vacuolar H⁺ ATPase (FIG. 5M). We next tested whether galectins can be in complexes with mTOR and its regulatory systems. Of the three included galectins (Gal3, Gal8 and Gal9), only Gal8 was found in co-IPs with mTOR and RagA (FIG. 3B). Gal8 was localized on the damaged lysosome upon GPN treatment (FIG. S3B). Additional components of the Ragulator-Rag system were found in complexes with Gal8 (FIG. S3C, D). Association between Gal and RagA increased upon treatment with GPN (FIG. 3C). This was also the case with Gal8 and Ragulator, since GPN treatment resulted in increased association between Gal8 and LAMTOR2/p14 (FIG. 3C). In contrast, GPN reduced levels of Raptor and mTOR in complexes with Gal8 (FIG. 3C).

To confirm these relationships we employed an in vivo proximity biotinylation assay using the engineered ascorbate peroxidase probe APEX2 (Lam, et al., 2015). This assay (Lam, et al., 2015) probes protein-protein proximity in vivo, whereby a protein's fusion with APEX2 preferentially biotinylates its immediate neighbors, due to short half-life and narrow labeling radius (<20 nm) of biotin-phenoxyl radical (the peroxidase reaction products of APEX2 with biotin-phenol) (Rhee, et al., 2013). APEX2 was fused at the N-terminus of Gal (and Gal9 as a control), cells transfected and treated with GPN, pulsed with biotin-phenol and H₂O, biotinylated products adsorbed to streptavidin beads in cell lysates, and proteins stripped from the beads and analyzed by immunoblotting. Using this assay, we found mTOR, Raptor and RagA in the proximity of Gal8 but not in the proximity of Gal3 or Gal9 (FIG. 3D). Lysosomal damage with GPN increased proximity of Gal to Ragulator (tested by immunoblotting for LAMTOR2/p14) and RagA and decreased proximity of Gal8 to mTOR and Raptor (FIG. 3E). GST-pulldown assays confirmed a capacity for direct interactions between Gal8 and Ragulator (using LAMTOR1/p18)(FIGS. 3F(i) and S3E(i)) and between GAL8 and all four Rag GTPases (FIGS. 3G(i)) and S3F(i-iv)). In contrast, Gal9, used as a comparator control in GST pull-downs, did not show direct binding to any of these proteins (FIGS. 3F(ii), G(ii), and S3E(ii)).

Gal8 showed in co-IPs higher associations with RagB^{T54L} (GDP, inactive RagB form) than with RagB^{Q99L} (GTP, constitutively active RagB form)(FIG. 3H), and similarly with RagA^{T21L} (GDP, inactive RagA form) than with RagA^{Q66L} (GTP, constitutively active RagA form) (FIG. S3G). Conversely, Gal8 co-IPs with RagC mutants indicated higher association of Gal8 with RagC^{Q20L} (GTP, constitutively active form) than with RagC^{S57L} (GDP, inactive form) (FIG. 3I). This is consistent with Gal8's preference for Rag GTPases reflecting mTOR inactivation.

These findings establish Gal8 association with mTOR and its regulators and reveal dynamic changes following lysosomal damage reflected in altered abundance of key components in protein complexes with Gal8 and changes in the proximity of mTOR components and its regulators relative to Ga. Following lysosomal damage, Gal8 is more firmly associated with Ragulator and RagA/B, whereas its proximity with mTOR and its adaptor Raptor lessens (FIG. S3H).

Galectin 8 is Required for mTOR Inactivation Upon Lysosomal Damage

To test whether Gal8 was functionally important in controlling mTOR, we generated a CRISPR Gal8 mutant in HeLa cells (Gal8KO^{HeLa}) (FIG. S4A). mTOR was not inactivated in Gal8KO^{HeLA} cells relative to the wild type (Gal8WT^{HeLa}) parental HeLa cells, assessed by S6K1 (pT389) and ULK1 (p-S757) phosphorylation levels (FIG. 4A). In contrast, response to starvation remained intact in Gal8KO^{HeLa} mutant cells (FIG. S4B, C). The defect in GPN-response in Gal8KO^{HeLa} cells was complemented by a full-size Gal8 construct (FIG. S4D). We next examined which domain(s) of Gal8 were important for Rag and Ragulator binding and found that of the two domains, termed carbohydrate recognition domains CRD1 and CRD2, CRD2 bound to Gal8 (FIG. S4E, F). GST-pulldown assays confirmed that CRD2 of Gal8 can directly bind RagA/C and RagB/D, tested as pairs, and LAMTOR1/p18, (FIG. S4G(i-iii)). However, the loss of GPN-response in Gal8KO^{HeLa} could not be fully complemented by either of the two Gal8 halves (CRD1 of CRD2) separately (FIG. S4D), indicating that binding was not sufficient to confer function and that a full size Gal8 was required for its effects on mTOR inactivation in response to lysosomal damage. Translocation of mTOR from lysosomes to the cytosol was diminished in Gal8KO^{HeLa} cells (FIG. 4B). As a further control, we knocked out Gal3 by CRISPR in HeLa cells (Gal3KO^{HeKa}) (FIG. S4H). Unlike Gal8KO^{HeLa}, the Gal3KO^{HeLa} cells responded to GPN treatment by reducing S6K1 (pT389) and ULK1 (p-S757) phosphorylation similarly to the parental HeLa cells (FIG. S4). Furthermore, downstream effector mechanisms such as autophagy, measured by levels of LC3-II (FIG. 4C), were not activated in Gal8KO^{HeLa} cells as readily as in Gal8WT^{HeLa} cells.

The inventors next tested primary cells using murine bone marrow-derived macrophages (BMMs) from Gal8 KO mice. BMMs underwent lysosomal damage upon exposure to GPN (higher concentrations, 400 μM, than in HeLa or 293T cells were necessary) or LLOMe as reflected in reduced LysoTracker Red DND-99 staining (FIG. S4J). As in Gal8KO^{HeLa} cells, a resistance to mTOR inactivation was detected in Gal8 KO BMMs (Gal8KO^{BMM}) vs. wild type BMMs (Gal8WT^{BMM}) (FIG. 4D). Induction of autophagy (negatively regulated by mTOR) in response to GPN, measured by LC3-II levels, was diminished in Gal8KO^{BMM} relative to Gal8WT^{BMM} (FIG. 4E), consistent with incomplete inactivation of mTOR. TFEB nuclear translocation, which is under negative control by mTOR, was not as prominent in Gal8KO^{BMM} relative to Gal8WT^{BMM} macrophages in response to GPN treatment (FIG. 4F). These findings indicate that Gal8 is a regulator of mTOR, that Gal8 is responsible in cells subjected to lysosomal damage for mTOR inactivation, and that this is reflected in downstream effector events (FIG. S4K). The inventors refer to and define galectins and their interactors involved in these processes as GALTOR, representing a dynamic galectin-based regulatory subsystem controlling mTOR, defined functionally herein as responding to lysosomal damage (FIG. S3H).

The Sensor SLC38A9 Interacts with Gal8

SLC38A9 is a lysosomal amino acid transporter that interacts with the Rag-Ragulator complex and is required for arginine from lysosomes to activate mTOR (Jung, et al., 2015; Rebsamen, et al., 2015; Wang, et al., 2015). Its sensory repertoire has recently been expanded to mTOR regulation in response to lysosomal cholesterol, independently of its arginine sensing functions (Castellano, et al., 2017). Since SLC38A9 appears to integrate diverse signaling inputs for mTOR at lysosomes, we tested if SLC38A9 might be involved in transducing Gal8 lysosomal damage signals to mTOR. SLC38A9 and Gal8 co-IPed, but only upon lysosomal damage, and did not associate in the resting state (full medium) or upon starvation (FIG. 5A). This suggests that Gal8 gains access to lumenal aspects of SLC38A9 including its lumenally exposed glycosylated groups (Wang, et al., 2015) following lysosomal membrane perturbation. We generated mutants in Gal8 residues known to perturb its ability to bind glycans (Stowell, et al., 2010: Gal8^{R69H} (changing the canonical Arg residue in CRD1 to His) and Gal8^{R232H} (changing a key Arg residue in CRD2 to His), and the double mutant Gal8^{R69H&R232H}. Only when both residues R69 and R232 were mutated in CRD1 and CRD2, was the GPN-induced association between Gal8 and SLC38A9 strongly diminished (FIG. 5B, C). Wild type Gal8 introduced into Gal8KO^{HeLa} cells complemented the loss of mTOR activity monitored by S6K phosphorylation, but Gal8^{R69H&R232H} did not (FIG. 5D). The Gal8^{R69H&R232H} mutant also displayed reduced recruitment to lysosomes following damage (FIG. S5A), albeit its translocation was not completely abrogated, indicating involvement of additional interactions besides carbohydrate recognition.

A question arose whether SLC38A9 was necessary for Gal8 interactions with Rags and Ragulator. The baseline interactions between Gal8 and RagB or LAMTOR1/p18 were unaffected in SLC38A9 knockout cells (FIG. 5E). However, increased interactions of Gal8 with RagB and LAMTOR1/p18 caused by lysosomal damage were not detectable in SLC38A9 knockout cells (FIG. 5E). This was accompanied by reduced recruitment of Gal8 to lysosomes in SLC38A9 knockout cells exposed to GPN compared to wild type cells (FIG. 5F). Thus, although SLC38A9 was not required for baseline Gal8-Rag/Ragulator interactions, it was needed for their enhanced association following lysosomal damage.

SLC38A9 is Required for mTOR Reactivation During Recovery from Lysosomal Damage

The specificity of binding between Gal8 and SLC38A9 and their interaction following lysosomal damage suggested the possibility that SLC38A9 might transduce lysosomal damage signals to mTOR regulatory machinery. If this were the case, a knockout in SLC38A9 might no longer respond to lysosomal injury by resisting further inhibition of mTOR activity. As expected (Jung, et al., 2015), basal mTOR activity was reduced in SLC38A9 KO cells (FIG. 6A), but it was further reduced when cells were subjected to lysosomal damage by GPN. Thus, inactivation of mTOR in response to lysosomal damage occurs even in the absence of SLC38A9. This was confirmed by examining autophagy. LC3-II levels and LC3 puncta increased robustly in response to GPN in SLC38A9 knockout cells as well as in control (wild type) cells (FIG. 6B).

However, SLC38A9 was required for return to normal mTOR activity upon GPN washout measured by S6K1 and ULK1 phosphorylation as well as by LC3-II levels (FIG. 6C). When SLC38A9 was overexpressed in 293T cells (FIG. 6D), GPN-induced inhibition of mTOR was prevented at the earlier time points during the time course, indicating that overexpressed SLC38A9 can interfere with the effects of lysosomal damage on mTOR inhibition, possibly by competition. These effects were confirmed by suppression of LC3-II levels and LC3 puncta in response to GPN in cells overexpressing SLC38A9 (FIG. 6D, E). Thus, although SLC38A9 is not required to transduce lysosomal damage-associated inhibitory signals to mTOR, it can counteract their input when overexpressed and is necessary for restoration of mTOR activity during recovery from lysosomal damage (FIG. S5B, C).

### Proteomic Proximity Analyses of Gal8 During Lysosomal Damage

The inventors next performed a bottom up proteomic analysis using liquid chromatography tandem mass spectrometry (LCMS/MS) in conjunction with proximity biotinylation with APEX2 (Hung, et al., 2016; Hung, et al., 2014) in cells expressing APEX2-Gal8 and treated or not treated with GPN. Three independent experiments with proximity biotinylation and LC/MS/MS, identified by spectral counting (Liu, et al., 2004) that SLC38A9 and RagA/B become proximal to APEX2-Gal8 (spectral counts increased in excess of 100-fold) in cells subjected to lysosomal damage by GPN (FIG. 6F, Table S1, tabs 1-3). The Ragulator component LAMTOR1/p18 was also identified as showing a large increase in its proximity to APEX2-Gal8 by spectral counts following treatment of cells with GPN (FIG. 6F, Table S1, tabs 1-3). In contrast, mTOR showed an inverse pattern, and was found by spectral counting in all three experiments as becoming more distal to APEX2-Gal8 (reflected in a decrease in spectral counts of >100-fold) in cells treated with GPN (FIG. 6F, Table S1, tabs 1-3). Two arbitrarily chosen proteins, CALCOCO2 (NDP52), a protein previously shown to bind Gal8 (Thurston et al., 2012) and HSP90 (HSP90α/β), were identified in 2/3 or 3/3 experiments, respectively, but the spectral counts did not change much (by comparison to SLC38A9, RagA/B, LAMTOR1/p18 and mTOR) with GPN treatment (FIG. 6F, Table S1, tabs 1-3). MS signal intensity of peptide precursor ions confirmed these relationships, i.e. a large increase in LAMTOR1/p18, SLC38A9, and RagA/B (>100-fold in each case), and a large decrease in mTOR proximity to APEX2-Gal8 following lysosomal damage with GPN (>00-fold)(Table S1, tab 4). No other identified proteins showed changes (in three experimental replicates ±GPN; Table S1) approaching magnitudes observed for SLC38A9, RagA/B, LAMTOR1/p18 on one end of the spectrum (increase), and mTOR on the opposite end of the spectrum (decrease)(Table St, tabs 1-4). These data confirm our identification of SLC38A9, LAMTOR1/p18 and RagA/B as the main candidates for Gal8 effectors in control of mTOR in response to lysosomal damage (FIG. 6G).

Galectin 9 Interacts with AMPK and Activates it During Lysosomal Damage

The inventors included AMPKα in our experiments as an anticipated negative control for galectin association. AMPKα was not detected in complexes with Gal3 and Gal8, but surprisingly, AMPKα was found in co-IPs with Gal9 (FIG. 7A). Unlike mTOR, which is inactivated with GPN, AMPK was activated by GPN as reflected in increased AMPKα T172 phosphorylation (FIG. 7B). This was accompanied by increased phosphorylation of AMPK's downstream targets (FIG. 7B). In cells treated with GPN, there was elevated phosphorylation of S79 within the critical metabolic enzyme acetyl-CoA carboxylase (ACC), and an increase in one of the activating AMPK phosphosites on ULK1 (pS317), but not of the mTOR-dependent inactivating phosphosite on ULK1, pS757, which was decreased (FIG. 7B).

The inventors generated a Gal9 KO in HEK293A cells using CRISPR (FIG. S5D), to test whether Gal9 was important for AMPK control. Gal9 KO abrogated the GPN-induced AMPK phosphorylation and the downstream pattern with ACC and ULK1 (FIG. 78). Gal9 KO cells retained increased AMPK phosphorylation in response to glucose starvation or oligomycin treatment (FIG. S5E, F). The defect in response to GPN was complemented by introducing FLAG-Gal9 into Gal9 KO HEK293A cells (FIG. S6A). A knockdown of Gal9 in HEK293T cells had a similar suppressive effect on AMPK phosphorylation response pattern elicited by lysosomal damage caused by LLOMe (FIG. S6B). When Gal9 was overexpressed in HEK293T cells, LLOMe-induced AMPK signature phosphorylation pattern was elevated (FIG. S6C). In cells knocked down for AMPKα, mTOR was partially resistant to inactivation by GPN (FIG. 7C), which fits the known AMPK-mTOR co-regulation circuitry and inhibition of mTOR by AMPK (Gwinn, et al., 2008; Shaw, et al., 2004).

We next tested the major kinases upstream of AMPK, LKB1 (Hawley, et al., 2003; Woods, et al., 2003), CaMMK2 (Hawley, et al., 2005; Woods, et al., 2005), and TAK1 (Herrero-Martin, et al., 2009) known to phosphorylate AMPKα at the T172 site. Only TAK1 was observed in co-IPs with Gal9 (FIG. 7D). Similarly to AMPKα, TAK1 interacted with Gal9 but not with Gal3 or Gal8 (FIG. S6D). Using biotinylation proximity assay with APEX2-Gal9, we also detected TAK1 but no LKB1 or CaMKK2 (FIG. 7E). Deletion mapping of Gal9 indicated that both Gal9's CRDs (CRD1 and CRD2) were contributing to full association between Gal9 with AMPKα, although CRD1retained partial binding capacity for AMPKα (FIG. S6E, F). This was paralleled by Gal9 CRD1's ability to associate fully with TAK1 (FIG. S6G). These results suggest that of the upstream kinases known to activate AMPK (Garcia and Shaw, 2017), TAK1 in association with Gal9 represented the best candidate for T172 phosphorylation and activation of AMPK in response to lysosomal damage. Accordingly, when the inventors knocked down TAK1, this abrogated AMPKα and ACC phosphorylation in response to GPN (FIG. S6H). However, we also observed a similar effect when LKB1 was knocked down, whereas CaMKK2 showed no effects (FIG. S6H). The observed contribution of LKB1 to AMPKα activation in response to lysosomal damage is compatible with a recently reported assembly of LKB1 and AMPK on lysosomes (Zhang, et al., 2014). The findings regarding Gal9-AMPK responses to lysosomal damage are summarized in FIG. S6I.

### Gal8 and Gal9 Coordinate Physiological Responses to Endomembrane Damage

A question arose whether both Gal8 and Gal9 are needed for autophagic response to lysosomal damage. We tested Gal9KO 293A cells and found a decreased autophagic response to GPN (FIG. 7F). Likewise, Gal8 KO in BMMs reduced autophagic response to lysosomal damage (FIG. 7G). Thus both Gal8 and Gal9 are important for an optimal autophagic response.

Among the biological outputs known to associate with autophagic activation is the control of intracellular Mycobacterium tuberculosis (Mtb)(Gutierrez, et al., 2004). Virulent Mtb (e.g. strain Erdman) can cause damage to endomembranes such as phagosomes or phagolysosomes (Manzanillo, et al., 2012). A protective role for Gal9 has already been established using Gal9 KO mice through an incompletely understood mechanism (Jayaraman, et al., 2010), and hence we asked whether Gal8 similarly to Gal9 contributed to protection against Mtb. When Gal8 KO mice were subjected to aerosol infection with Mth Erdman, Gal8 KO animals showed increased susceptibility relative to wild type littermates (FIG. 7H). In conclusion, Gal8 and Gal9 jointly orchestrate physiological responses to endomembrane/lysosomal damage.

### Discussion

The present invention and experiments described herein shows that mTOR and AMPK are coordinately regulated by lysosomal damage, that specific galectins that recognize lysosomal damage associate with these regulators of cellular metabolism, and that Gal8 inhibits mTOR in response to non-metabolic inputs such as loss of endomembrane integrity. The galectin-containing complexes, functionally defined as a subsystem converging upon and controlling mTOR, are collectively referred to here as GALTOR. GALTOR response results in reprogramming of downstream effectors, i.e. S6K, and ULK1, as parts of anabolic and catabolic pathways, and includes autophagy, which represents both a metabolic pathway and a protein and membrane cytoplasmic quality control process. Whereas mTOR and AMPK are established as regulators of autophagy in its metabolic function (Garcia and Shaw, 2017; Saxton and Sabatini, 2017, their engagement in activating autophagy as an intracellular organelle and protein quality control pathway has not been as intuitive or established. The present work closes this gap and assigns a non-metabolic, membrane-homeostatic role to mTOR and AMPK as one of their key functions.

When lysosomal membranes are injured, Gal8 suppresses mTOR activity through its Ragulator-Rag signaling machinery (Saxton and Sabatini, 2017), whereas Gal9 activates AMPK possibly through recruitment of its known upstream activator TAK (Herrero-Martin, et al., 2009). In addition to TAK1, the LKB1-AMPK axis plays a role, recently reported to respond to disruptions in lysosomal v-ATPase (Zhang, et al., 2014). Since AMPK inhibits mTOR via Raptor, TSC2 (Carroll, et al., 2016; Demetriades, et al., 2014; Saxton and Sabatini, 2017 and additional mechanisms (Zhang, et al., 2014), the Gal9-driven activation of AMPK and Gal8-driven inactivation of mTOR upon lysosomal damage sets off a harmonized set of effects on AMPK and mTOR.

Galectins are intriguing proteins synthesized as cytosolic entities and released extracellularly (Arthur, et al., 2015). Galectins' intracellular functions have been less understood. In autophagy, galectins have been implicated primarily as“tags” for damaged membranes to guide their selective autophagy (Chauhan, et al., 2016; Thurston, et al., 2012). Gal8 interacts with NDP52 in the selective autophagy process termed xenophagy (Thurston, et al., 2012). However, Gal8's partner NDP52 is dispensable for mTOR inactivation since murine macrophages, which lack NDP52, are responsive to lysosomal damage. Instead, galectins through GALTOR directly control mTOR and AMPK.

The same core machinery regulating mTOR at the lysosome in response to nutrients (Castellano, et al., 2017; Saxton and Sabatini, 2017) is engaged in transducing lysosomal damage. The following model emerges (FIG. S3H). In the resting state, Gal8 is in complexes with mTOR. Following lysosomal damage, Gal8 increases interactions with the Ragulator-Rag/SLC3A9 apparatus while its association with mTOR and Raptor lessens. This is consistent with mTOR's translocation from the lysosome to the cytosol caused by lysosomal damage, whereas the Ragulator-Rag complex retained on the lysosome increases its interactions with Gal. A reflection of these events is the preferential association of Gal8 with the inactive (GDP) form of RagA. In parallel, AMPK is activated through a Gal9-dependent pathway.

It is important to understand the effects driven by lysosomal/endomembrane damage on general metabolism. This connects organically with the roles of mTOR and AMPK in immunometabolism (O'Neill, et al., 2016). In a good fit with this, the process of autophagy, which has numerous immunological functions (Deretic, et al., 2013), is predominantly anti-inflammatory (Deretic and Levin, 2018) congruent with the pattern of inactive mTOR and active AMPK in anti-inflammatory and immunological memory polarization states (O'Neill, et al., 2016). The integration of metabolic processes with responses to membrane damage uncovered herein should provide impetus to study roles of autophagy, mTOR and AMPK in immunometabolism and, conversely, to study the role of these metabolic systems in endomembrane and cytoplasmic quality control of relevance for aging, cancer, and a broad spectrum of diseases. The links between lysosomal damage and mTOR inhibition, AMPK activation, and autophagy induction, may explain the curious placement of mTOR and its regulators at the lysosome (Saxton and Sabatini, 2017, reflecting not only metabolic needs but also innate defenses against invading microbes, e.g. Mtb studied here and elsewhere (Gutierrez. Et al., 2004, or exogenous and endogenous agents (Deretic and Levin, 2018) of sterile endomembrane injury and inflammation.

### REFERENCES

Aits, S., Kricker, J., Liu, B., Ellegaard, A. M., Hamalisto, S., Tvingsholm, S., Corcelle-Termeau, E., Hogh, S., Farkas, T., Holm Jonassen, A., et al. (2015). Sensitive detection of lysosomal membrane permeabilization by lysosomal galectin puncta assay. Autophagy 11, 1408-1424.
Arthur, C. M., Baruffi, M. D., Cummings, R. D., and Stowell, S. R. (2015). Evolving mechanistic insights into galect infunctions. Methods Mol Biol 1207, 1-35.
Bar-Peled, L., Schweitzer, L. D., Zoncu, R., and Sabatini, D. M. (2012). Ragulator is a GEF for the rag GTPases that signal amino acid levels to mTORC1. Cell 150, 1196-1208.
Berg, T. O., Stromhaug, E., Lovdal, T., Seglen, O., and Berg, T. (1994). Use of glycyl-L-phenylalanine 2-naphthylamide, a lysosome-disrupting cathepsin C substrate, to distinguish between lysosomes and prelysosomal endocytic vacuoles. Biochem J 300 (Pt 1), 229-236.
Betz, C., and Hall, M. N. (2013). Where is mTOR and what is it doing there? J Cell Biol 203 , 563-574.
Carroll, B., Maetzel, D., Maddocks, O. D., Otten, G., Ratcliff, M., Smith, G. R., Dunlop, E. A., Passos, J. F., Davies, O. R., Jaenisch, R., et al. (2016). Control of TSC2-Rheb signaling axis by arginine regulates mTORC1 activity. eLife 5.
Castellano, B. M., Thelen, A. M., Moldavski, O., Feltes, M., van der Welle, R. E., Mydock-McGrane, L., Jiang, X., van Eijkeren, RJ., Davis, O. B., Louie, S. M., et al. (2017). Lysosomal cholesterol activates mTORC1 via an SLC38A9-Niemann-Pick C1 signaling complex. Science 355, 1306-1311.
Chauhan, S., Kumar, S., Jain, A., Ponpuak, M., Mudd, M., Kimura, T., Choi, S. W., Peters, R., Mandell, M., Bruun, J. A., et al. (2016). TRIMs and Galectins Globally Cooperate and TRIM16 and Galectin-3 Co-direct Autophagy in Endomembrane Damage Homeostasis. Dev Cell 39, 13-27.
Demetriades, C., Doumpas, N., and Teleman, A. A. (2014). Regulation of TORC1 in response to amino acid starvation via lysosomal recruitment of TSC2. Cell 156, 786-799.
Deosaran, E., Larsen, K. B., Hua, R., Sargent, G., Wang, Y., Kim, S., Lamark, T., Jauregui, M., Law, K., Lippincott-Schwartz, J., et al. (2013). NBR1 acts as an autophagy receptor for peroxisomes. J Cell Sci 126, 939-952.
Deretic, V., and Levine, B. (2018). Autophagy balances inflammation in innate immunity. Autophagy, 1-9.
Deretic, V., Saitoh, T., and Akira, S. (2013). Autophagy in infection, inflammation and immunity. Nat Rev Immunol 13, 722-737.
Fujita, N., Morita, E., Itoh, T., Tanaka, A., Nakaoka, M., Osada, Y., Umemoto, T., Saitoh, T., Nakatogawa, H., Kobayashi, S., et al. (2013). Recruitment of the autophagic machinery to endosomes during infection is mediated by ubiquitin. J Cell Biol 203, 115-128.
Garcia, D., and Shaw, RJ. (2017). AMPK: Mechanisms of Cellular Energy Sensing and Restoration of Metabolic Balance. Mol Cell 66, 789-800.
Gutierrez, M. G., Master, S. S., Singh, S. B., Taylor, G. A., Colombo, M. I., and Deretic, V. (2004). Autophagy is a defense mechanism inhibiting BCG and Mycobacterium tuberculosis survival in infected macrophages. Cell 119, 753-766.
Gwinn, D. M., Shackelford, D. B., Egan, D. F., Mihaylova, M. M., Mery, A., Vasquez, D. S., Turk, B. E., and Shaw, R. J. (2008). AMPK phosphorylation of raptor mediates a metabolic checkpoint. Mol Cell 30, 214-226.
Hawley, S. A., Boudeau, J., Reid, J. L., Mustard, K. J., Udd, L., Makela, T. P., Alessi, D. R., and Hardie, D. G. (2003). Complexes between the LKB1 tumor suppressor, STRAD alpha/beta and MO25 alpha/beta are upstream kinases in the AMP-activated protein kinase cascade. Journal of Biology 2, 28.
Hawley, S. A., Pan, DA., Mustard, K. J., Ross, L., Bain, J., Edelman, A. M., Frenguelli, B. G., and Hardie, D. G. (2005). Calmodulin-dependent protein kinase kinase-beta is an alternative upstream kinase for AMP-activated protein kinase. Cell Metabolism 2, 9-19.
Herrero-Martin, G., Hoyer-Hansen, M., Garcia-Garcia, C., Fumarola, C., Farkas, T., Lopez-Rivas, A., and Jaattela, M. (2009). TAK1 activates AMPK-dependent cytoprotective autophagy in TRAIL-treated epithelial cells. The EMBO journal 28, 677-685.
Hornung, V., Bauernfeind, F., Halle, A., Samstad, E. O., Kono, H., Rock, K. L., Fitzgerald, K. A., and Latz, E. (2008). Silica crystals and aluminum salts activate the NALP3 inflammasome through phagosomal destabilization. Nat Immunol 9, 847-856.
Hung, V., Udeshi, N. D., Lam, S. S., Loh, K. H., Cox, K. J., Pedram, K., Carr, S. A., and Ting, A. Y. (2016). Spatially resolved proteomic mapping in living cells with the engineered peroxidase APEX2. Nat Protoc 11, 456-475.
Hung, V., Zou, P., Rhee, H. W., Udeshi, N. D., Cracan, V., Svinkina, T., Carr, S. A., Mootha, V. K., and Ting, A. Y. (2014). Proteomic mapping of the human mitochondrial intermembrane space in live cells via ratiometric APEX tagging. Mol Cell 55, 332-341.
Inoki, K., Li, Y., Xu, T., and Guan, K. L. (2003). Rheb GTPase is a direct target of TSC2 GAP activity and regulates mTOR signaling. Genes Dev 17, 1829-1834.
Jayaraman, P., Sada-Ovalle, I., Beladi, S., Anderson, A. C., Dardalhon, V., Hotta, C., Kuchroo, V. K., and Behar, S. M. (2010). Tim3 binding to galectin-9 stimulates antimicrobial immunity. J Exp Med 207, 2343-2354.
Johansen, T., and Lamark, T. (2011). Selective autophagy mediated by autophagic adapter proteins. Autophagy 7, 279-296.
Jung, J., Genau, H. M., and Behrends, C. (2015). Amino Acid-Dependent mTORC1 Regulation by the Lysosomal Membrane Protein SLC38A9. Mol Cell Bio 35, 2479-2494.
Karanasios, E., Walker, S. A., Okkenhaug, H., Manifava, M., Hummel, E., Zimmermann, H., Ahmed, Q., Domart, M. C., Collinson, L, and Ktistakis, N. T. (2016). Autophagy initiation by ULK complex assembly on ER tubulovesicular regions marked by ATG9 vesicles. Nat Comm 7, 12420.
Khaminets, A., Heinrich, T., Mari, M., Grumati, P., Huebner, A. K., Akutsu, M., Liebmann, L., Stolz, A., Nietzsche, S., Koch, N., et al. (2015). Regulation of endoplasmic reticulum turnover by selective autophagy. Nature 522, 354-358.
Kim, E., Goraksha-Hicks, P., Li, L., Neufeld, T. P., and Guan, K. L. (2008). Regulation of TORC1 by Rag GTPases in nutrient response. Nature Cell Biol 10, 935-945.
Kim, J., Kundu, M., Viollet, B., and Guan, K. L. (2011). AMPK and mTOR regulate autophagy through direct phosphorylation of Ulk1. Nat Cell Biol 13, 132-141.
Lam, S. S., Marteli, J. D., Kamer, K. J., Deerinck, T. J., Ellisman, M. H., Mootha, V. K., and
Ting, A. Y. (2015). Directed evolution of APEX2 for electron microscopy and proximity labeling. Nature Methods 12, 51-54.
Lazarou, M., Sliter, D. A., Kane, L A., Sarraf, S. A., Wan& C., Burman, J. L., Sideris, D. P., Fogel, A. I., and Youle, R. J. (2015). The ubiquitin kinase PINK1 recruits autophagy receptors to induce mitophagy. Nature 524, 309-314.
Liu, H., Sadygov, R. G., and Yates, J. R., 3rd (2004). A model for random sampling and estimation of relative protein abundance in shotgun proteomics. Anal Chem 76, 4193-4201.
Long, X., Lin, Y., Ortiz-Vega, S., Yonezawa, K., and Avruch, (2005). Rheb binds and regulates the mTOR kinase. Current biology 15, 702-713.
Manifava, M., Smith, M., Rotondo, S., Walker, S., Niewczas, I., Zoncu, R., Clark, J., and Ktistakis, N. T. (2016). Dynamics of mTORC1 activation in response to amino acids. Elife 5.
Manzanillo, P. S., Shiloh, M. U., Portnoy, D. A., and Cox, J. S. (2012). Mycobacterium Tuberculosis Activates the DNA-Dependent Cytosolic Surveillance Pathway within Macrophages. Cell Host & Microbe 11, 469-480.
Mizushima, N., Yoshimori, T., and Ohsumi, Y. (2011). The role of atg proteins in autophagosome formation. Ann Rev Cell Dev Bio 27, 107-132.
Napolitano, G., and Ballabio, A. (2016). TFEB at a glance. J Cell Sci 129, 2475-2481.
O'Neill, L. A., Kishton, R. J., and Rathmell, J. (2016). A guide to immunometabolism for immunologists. Nat Rev Immunol 16, 553-565.
Rebsamen, M., Pochini, L., Stasyk, T., de Araujo, M. E., Galluccio, M., Kandasamy, R. K., Snijder, B., Fauster, A., Rudashevskaya, E. L, Bruckner, M., et al. (2015). SLC38A9 is a component of the lysosomal amino acid sensing machinery that controls mTORC. Nature 519, 477-481.
Rhee, H. W., Zou, P., Udeshi, N. D., Martell, J. D., Mootha, V. K., Carr, S. A., and Ting, A. Y. (2013). Proteomic mapping of mitochondria in living cells via spatially restricted enzymatic tagging. Science 339, 1328-1331.
Sancak, Y., Bar-Peled, L, Zoncu, R., Markhard, A. L., Nada, S., and Sabatini, D. M. (2010). Ragulator-Rag complex targets mTORC to the lysosomal surface and is necessary for its activation by amino acids. Cell 141, 290-303.
Sancak, Y., Peterson, T. R., Shaul, Y. D., Lindquist, R. A., Thoreen, C. C., Bar-Peled, L., and Sabatini, D. M. (2008). The Rag GTPaees bind raptor and mediate amino acid signaling to mTORCL. Science 320, 1496-1501.
Sancak, Y., Thoreen, C. C., Peterson, T. R., Lindquist, R. A., Kang, S. A., Spooner, E., Carr, S. A., and Sabatini, D. M. (2007). PRAS40 is an insulin-regulated inhibitor of the mTORC protein kinase. Mol Cell 25, 903-915.
Saxton, R. A., and Sabatini, D. M. (2017). mTOR Signaling in Growth, Metabolism, and Disease. Cell 168, 960-976.
Shaw, R J., Bardeesy, N., Manning, B. D., Lopez, L., Kosmatka, M., DePinho, R. A., and Cantley, LC. (2004). The LKB1 tumor suppressor negatively regulates mTOR signaling. Cancer Cell 6, 91-99.
Stowell, S. R., Arthur, C. M., Dias-Baruffi, M., Rodrigues, L. C., Gourdine, J. P., Heimburg-Molinaro, J., Ju, T., Molinaro, R. J., Rivera-Marrero, C., Xia, B., et al. (2010). Innate immune lectins kill bacteria expressing blood group antigen. Nature Medicine 16, 295-301.
Tee, A. R., Manning, B. D., Roux, P. P., Cantley, L. C., and Blenis, J. (2003). Tuberous sclerosis complex gene products, Tuberin and Hamartin, control mTOR signaling by acting as a GTPase-activating protein complex toward Rheb. Current biology 13, 1259-1268.
Thiele, D. L., and Lipsky, P. E. (1990). Mechanism of L-leucyl-L-leucine methyl ester-mediated killing of cytotoxic lymphocytes: dependence on a lysosomal thiol protease, dipeptidyl peptidase, that is enriched in these cells. Proc Nat Acad Sci USA 87, 83-87.
Thurston, T. L., Wandel, M. P., von Muhlinen, N., Foeglein, A., and Randow, F. (2012). Galectin 8 targets damaged vesicles for autophagy to defend cells against bacterial invasion. Nature 482, 414-418.
Wang, S., Tsun, Z. Y., Wolfson, R. L., Shen, K., Wyant, G. A., Plovanich, M. E., Yuan, E. D., Jones, T. D., Chantranupong, L., Comb, W., et al. (2015). Metabolism. Lysosomal amino acid transporter SLC38A9 signals arginine sufficiency to mTORC1. Science 347, 188-194.
Woods, A., Dickerson, K., Heath, R., Hong, S. P., Momcilovic, M., Johnstone, S. R., Carlson, M., and Carling, D. (2005). Ca2+/calmodulin-dependent protein kinase kinase-beta acts upstream of AMP-activated protein kinase in mammalian cells. Cell metabolism 2, 21-33.
Woods, A., Johnstone, S. R., Dickerson, K., Leiper, F. C., Fryer, L. G., Neumann, D., Schlattner, U., Wallimann, T., Carlson, M., and Carling, D. (2003). LKB1 is the upstream kinase in the AMP-activated protein kinase cascade. Current biology 13, 2004-2008.
Zhang, C. S., Jiang, B., Li, M., Zhu, M., Peng, Y., Zhang, Y. L., Wu, Y. Q., Li, T. Y., Liang, Y.,
Lu, Z., et al. (2014). The lysosomal v-ATPase-Ragulator complex is a common activator for AMPK and mTORC1, acting as a switch between catabolism and anabolism. Cell Metabolism 20, 526-540.
Zhang, J., Tripathi, D. N., Jing, J., Alexander, A., Kim, J., Powell, R. T., Dere, R., Tait-Mulder, J., Lee, J. H., Paull, T. T., et al. (2015). ATM functions at the peroxisome to induce pexophagy in response to ROS. Nat Cell Biol 17, 1259-1269.
Zoncu, R., Bar-Peled, L., Efeyan, A., Wang, S., Sancak, Y., and Sabatini, D. M. (2011). mTORC1 senses lysosomal amino acids through an inside-out mechanism that requires the vacuolar H(+)-ATPase. Science 334, 678-683.

1. A method of treating an autophagy mediated disease in a patient in need comprising administering to said patient an effective amount of Galectin-8 and/or Galectin-9, a modulator/upregulator of Galectin-8 and/or Galectin-9, or an agent which acts similar to Galectin-8 as an inhibitor of mTOR and/or Galectin-9 as a modulator (upregulator) of AMPKinase or a mixture thereof, optionally in combination with a lysosomotropic agent.

2. The method according to claim 1 wherein said upregulator of galectin-8 or Galectin-9 or said agent which acts similarly to Galectin-8 and/or Galectin-9 is a sugar which comprises at least one galactose unit.

3. The method according to claim 2 wherein said sugar is selected from the group consisting of a monosaccharide, including O-galactoside sugars, such as galactose, including N- or O-linked galactosides and disaccharides, oligosaccharides and polysaccharides which contain at least one galactose unit.

4. The method according to claim 2 wherein said sugar is galactose, a galactoside, lactose, mannobiose, melibiose, melibiulose (which may have the galactose residue optionally N-acetylated), rutinose, rutinulose, xylobiose, trehalose, or a mixture thereof all of which optionally comprise N and O-linked acetyl groups.

5. The method according to claim 3 wherein said sugar is an oligosaccharide containing at least one galactose unit.

6. (canceled)

7. The method according to claim 2 wherein said sugar is a galactoside or is a galactose derivative.

8. The method according to claim 1 wherein said agent which acts similar to Galectin-8 or Galectin-9 or upregulates Galectin-8 or Galectin-9 is a lactulose amine such as N-lactulose-octamethylenediamine (LDO); N,N-dilactulose-octamethylenediamine (D-LDO), and N,N-dilactulose-dodecamethylenediamine (D-LDD)), OR-MD-02, ipilimumab, a pectin, or a taloside inhibitor.

9. The method according to claim 1 wherein said composition includes a lysosomotropic agent.

10. (canceled)

11. The method according to claim 9 wherein said lysosomotropic agent is a lysosomotropic detergent.

12. The method according to claim 11 wherein said lysosomotropic detergent is a lysosomotropic amine containing a moderately basic amine of pKa 5-9.

13. The method according to claim 12 wherein said lysosomotropic amine is sphingosine, O-methyl-serine dodecylamine hydrochloride (MSDH), N-dodecylimidazole, or a mixture thereof.

14. The method according to claim 9 wherein said lysomotropic agent is chloroquine, chlorpromazine, thioridazine, aripiprazole, clomipramine, imipramine, desipramine, seramasine, or a mixture thereof.

15. The method according to claim 9 wherein said lysosomotropic agent is glycyl-L-phenylalanine-2-naphthyl amide (GPN), Leu-Leu-OMe (LLOMe) or a mixture thereof.

16. The method according to claim 1 wherein said autophagy mediated disease state is a metabolic syndrome disease, a microbial infection, an inflammatory disorder, a lysosomal storage disorder, an immune disorder, cancer or a neurodegenerative disorder.

17. The method according to claim 16 wherein said microbial infection is a Mycobacterium infection.

18. (canceled)

19. (canceled)

20. (canceled)

21. The method according to claim 1 further including administering at least one additional agent selected from the group consisting of an additional autophagy modulator and/or at least one compound selected from the group consisting of Torin, pp242, rapamycin/serolimus (which also may function as an autophagy modulator), everolimus, temsirolomis, ridaforolimis, zotarolimis, 32-dexoy-rapamycin, epigallocatechin gallate (EGCG), caffeine, curcumin, reseveratrol or mixtures thereof.

22. The method according to claim 1 wherein said autophagy mediated disease state is a metabolic syndrome disease, an infectious disease, a lysosome storage disease, cancer or an aging related disease or disorder.

23. The method according to claim 1 wherein said autophagy mediated disease state is Alzheimer's disease, Parkinson's disease, Huntington's disease; inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (I and II), severe insulin resistance, hyperinsulinemia, insulin-resistant diabetes, dyslipidemia depressed high-density lipoprotein (HDL), and elevated triglycerides, liver disease, renal disease, cardiovascular disease, including infarction, ischemia, stroke, pressure overload and complications during reperfusion, muscle degeneration and atrophy, symptoms of aging, low grade inflammation, gout, silicosis, atherosclerosis, age-associated dementia and sporadic form of Alzheimer's disease, psychiatric conditions including anxiety and depression, spinal cord injury, arteriosclerosis or a bacterial, fungal, cellular or viral infections.

24. The method according to claim 1 wherein said autophagy mediated disease state is activator deficiency/GM2 gangliosidosis, alpha-mannosidosis, aspartylglucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM Ganliosidosis, including infantile, late infantile/juvenile and adult/chronic), Hunter syndrome (MPS II), I-Cell disease/Mucolipidosis II, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs or Wolman disease.

25-27. (canceled)

28. A pharmaceutical composition comprising an effective amount of Galectin-8 and/or Galectin-9, a modulator/upregulator of Galectin-8 and/or Galectin-9, or an agent which acts similar to Galectin-8 as an inhibitor of mTOR and/or Galectin-9 as a modulator (upregulator) of AMPKinase or a mixture thereof, optionally in combination with a lysosomotropic agent.

29-43. (canceled)
